(19) United States
(12) Patent Application Publication
(10) Pub. No.: US 2013/0317083 A1
(43) Pub. Date: Nov. 28, 2013

(54) NON-CODING TRANSCRIPTS FOR DETERMINATION OF CELLULAR STATES

(71) Applicant: THOMAS JEFFERSON UNIVERSITY, Philadelphia, PA (US)

(72) Inventor: Isidore Rigoutsos, Astoria, NY (US)

(73) Assignee: THOMAS JEFFERSON UNIVERSITY, Philadelphia, PA (US)

(21) Appl. No.: 13/886,750

(22) Filed: May 3, 2013

Related U.S. Application Data
(60) Provisional application No. 61/642,802, filed on May 4, 2012.

(51) Int. Cl. C12Q 1/68

(52) U.S. Cl. CPC C12Q 1/6886; C12Q 1/6883
USPC 514/44 A; 435/6.14; 435/287.2; 435/6.16; 435/6.12

(57) ABSTRACT

Disclosed herein are novel methods, assays and systems for determining a given state of a cell or a tissue by detecting the presence or absence of a short RNA molecule originating from (a) at least one or more exons of at least one or more protein-coding genes, or from (b) at least one or more segments of at least one or more non-coding transcripts, or from (c) both (a) and (b), in a biological sample from a subject. In some embodiments, the methods, assays and systems described herein can be used to identify an origin and/or a type of a cell or tissue, and/or distinguish a cell or tissue from another cell or tissue. In some embodiments, the methods, assays and systems described herein can also be used to diagnose a disease or disorder, or prognose a given stage and/or progression of the disease or disorder in a subject.

### CROSS-REFERENCE TO RELATED APPLICATIONS

This application claims benefit under 35 U.S.C. §119(e) of U.S. Provisional Application No. 61/642,802 filed on May 4, 2012, the content of which is incorporated herein by reference in its entirety.

### TECHNICAL FIELD

Provided herein relates to methods for determining a cellular state or a tissue state of a biological sample. Specifically, some embodiments of the methods described herein can be used to diagnose or prognose for a given stage of a disease, e.g., cancer, or disorder, in a subject.

### BACKGROUND OF THE DISCLOSURE

Cancer is a leading cause of death worldwide. According to the World Health Organization (WHO), cancer accounts for about 13% of all deaths (about 7.6 million deaths) in 2008. However, if the cancer is diagnosed early or prognosed correctly, appropriate treatment can start earlier in the disease process and can generally have a higher rate of success.

Numerous different classifications of the clinical disease stages have been used for cancer. Common elements considered in cancer stage or grade classification include, for example, site of the primary tumor, tumor size and number of tumors, lymph node involvement (spread of cancer into lymph nodes), and cell type and morphology (how closely cancer cells resemble normal tissue cells or malignant cells), and the presence or absence of metastasis.

To diagnose and/or prognose cancer, imaging tools or laboratory tests are commonly used. For example, imaging tools, such as X-rays, computed tomography (CT) scans, magnetic resonance imaging (MRI) scans, and positron emission tomography (PET) scans are used to visualize a tumor and its spread. Laboratory tests such as blood or urine tests are used to detect the presence or absence of a cancer biomarker. However, to determine a given stage of cancer, e.g., ductal carcinoma in situ vs. invasive tumor, pathology/histology tests based on a biopsy is still the gold standard. Yet interpretation of the pathology test result can be biased by subjective criteria, poor technical skills and/or pathologists' experience. As such, there is still a strong need for more efficient and/or reliable methods to determine a given stage of a disease, e.g., cancer, or disorder.

### SUMMARY

Pathology is still currently the gold standard for diagnosis of various diseases such as cancer or disorders, and/or determination of a given stage of a disease or disorder. However, proper technical skills for processing a biopsy sample and experienced pathologists are critical, or difficult interpretation problems can arise. Thus, there is still a strong need to develop methods that are more definitive and reliable for diagnosing or determining a given stage of a disease such as cancer or disorder. While it is generally known that exons of a protein-coding region make up a transcript, the mRNA, which is translated by a ribosome into an amino acid sequence or protein, the inventor has surprisingly discovered that one or more exons of a protein-coding gene can give rise to one or more short RNA molecules. Furthermore, the presence and/or amount of these short RNA molecules and/or the exact location of their origin/source in the corresponding exons can be indicative of the state of a cell and/or a tissue (e.g., normal vs. diseased or abnormal tissue). Additionally or alternatively, the presence and/or amount of short RNA molecules and/or the exact location of their origin/source in the corresponding exons can depend on a given state of a disease (e.g. ductal-in-situ-carcinoma tissue vs. invasive carcinoma tissue) or disorder. Accordingly, provided herein relates to methods, assays and systems for determining a given state of a cell and/or a tissue, which can be used for diagnosing a disease or disorder, and/or prognosing a given stage and/or progression of a disease or disorder.

In one aspect, provided herein relates to methods or assays of determining a given state of a cell and/or a tissue. The method or assay comprises detecting in a biological sample the presence or absence of a short RNA sequence originating from (a) an exon of at least one protein-coding gene; or (b) a segment of at least one non-coding transcript; or (c) both (a) and (b). In some embodiments, the biological sample can be derived from a subject suspected of being at risk of or having a given stage of a disease or disorder. Accordingly, methods or assays described herein can also be used to determine whether a subject has, or is at risk of developing, or is at a given stage of a disease or disorder, e.g., a condition afflicting a tissue of interest. In one embodiment, the condition afflicting a tissue of interest includes cancer.

In some embodiments, the method or assay described herein can comprise detecting in the biological sample the presence or absence of a plurality of short RNA sequences originating from an exon of at least one protein-coding gene, and/or from a segment of at least one non-coding transcript. In some embodiments, the plurality of the short RNA sequences can originate from more than one exons of at least one protein-coding gene. In some embodiments, the plurality of the short RNA sequences can originate from more than one segments of at least one non-coding transcript.

In some embodiments or other embodiments of any aspects described herein, a short RNA sequence is at least a segment of an exon of a protein-coding gene. Without limitations, the region of focus can include an amino acid coding sequence or an untranslated region of the protein-coding gene, e.g., 3′ untranslated region (3′ UTR) or 5′ untranslated region (5′ UTR). In some embodiments or other embodiments of any aspects described herein, a short RNA sequence is a segment of a non-coding transcript.

The short RNA sequences detected herein can have a length of about 5 nucleotides to about 200 nucleotides, or about 5 nucleotides to about 100 nucleotides. In some embodiments, the short RNA sequences can have a length of about 10 nucleotides to about 100 nucleotides. In one embodiment, the short RNA sequences can have a length of about 10 nucleotides to about 50 nucleotides. In one embodiment, the short RNA sequences can have a length of about 10 nucleotides to about 40 nucleotides. In some embodiments, the short RNA sequences can have a length of about 32 nucleotides to about 50 nucleotides or about 32 nucleotides to about 40 nucleotides. In one embodiment, the short RNA sequences can have a length of about 34 nucleotides. In one embodiment, the short RNA sequence does not bind to mRNA. In some embodiments, the short RNA sequence is not an miRNA. In some embodiments, the short RNA sequence is not a piRNA. In some embodiments, the short sequence is not a siRNA. By way of example only, exemplary short RNA sequences originating from one or more exons of the protein-coding gene ELOVL5 can include, but are not limited to, AAATCTAGTGGAACAGTCAGTTTAACTTTTTAAC (SEQ ID NO: 1) or TTACTATGGTTTGTCGTCAGTCCCTTCCATGCGT (SEQ ID NO: 2) or can include fragments of these sequences, or can includes these sequences as substrings.

Other exemplary short RNA sequences originating from exons of the protein-coding gene ELOVL5 can include, but are not limited to, ATGTGAAATCAGACACGGCACCTTCA (SEQ ID NO: 3), AAATCTAGTGGAACAGTCAGTTTAACTTTTTAAC (SEQ ID NO: 4), ATTTGAGGCAGTGGTCAAACAGGTAAAGC (SEQ ID NO: 5), TATGAGTTGTGCCCCAATGC (SEQ ID NO: 6), TACAATGTTGTTATGGTAGAGAAACACACATGCC (SEQ ID NO: 7), CTATTGGCTTTGAATCAAGCAGGCTC (SEQ ID NO: 8), TGTATGTCTTCATTGCTAGG (SEQ ID NO: 9), TCCAAACCACGTCATCTGATTGTAAGCA (SEQ ID NO: 10), GCCTATGATGTGTGTCATTTTAAAGTGTCGGA (SEQ ID NO: 11), CACGTCATCTGATTGTAAGCAC (SEQ ID NO: 12), AAGCTGCGGAAGGATTGAAGTCAAAGAATT (SEQ ID NO: 13), TAAAGCCTATGATGTGTGTCATTT (SEQ ID NO: 14), GGGTCTAAATTTGGATTGATTTATGCAC (SEQ ID NO: 15), AGATTTCTAACATTTCTGGGCTCTCTGACC (SEQ ID NO: 16), AAGCAAAGTGTAAATCAGAGGTTTAAGTTAAAAT (SEQ ID NO: 17), TGATTCATGTAGGACTTCTTTCATCAATTCAAAA (SEQ ID NO: 18), GTGTCATTTTAAAGTGTCGGAATTTAGCCTCT (SEQ ID NO: 19), GTGGGTTTTCTGTTTGAAAAGGAG (SEQ ID NO: 20), GACACGGCACCTTCAGTTTTGTACTAT (SEQ ID NO: 21), CATAAGAGAATCGAGAAATTTGATAGAGGT (SEQ ID NO: 22), CAGCATAAGAGAATCGAGAAA (SEQ ID NO: 23), AAGCTTATTAGTTTAAATTAGGGTATGTTTC (SEQ ID NO: 24), TGTCTAAACAGTAATCATTAAAACATTTTTGATT (SEQ ID NO: 25), TAGACTGCTTATCATAAAATCACATC (SEQ ID NO: 26), CTTAGCTCACCTGGATATAC (SEQ ID NO: 27), CGTAGATGAGCAATGGGGAAC (SEQ ID NO: 28), ATGTAGGACTTCTTTCATCAATTCAAAACC (SEQ ID NO: 29), ATGCTTTAATTTTGCACATTCGTACTATAGGGAG (SEQ ID NO: 30), ATAAGATTTCTAACATTTCTGGGCTCTCTGACCC (SEQ ID NO: 31), AGGTAAAATCAAATATAGCTACAGC (SEQ ID NO: 32), AGAGATGATTGCCTATTTACC (SEQ ID NO: 33), AACCCCTAGAAAACGTATAC (SEQ ID NO: 34), AACATTTCTGGGCTCTCTGACCCCTGCG (SEQ ID NO: 35), TTATCATAAAATCACATCTCACACATTTGAGGC (SEQ ID NO: 36), TGGATATACCTACATTGTTAAATGTC (SEQ ID NO: 37), TGCTTTAATTTTGCACATTCGTACTATAGGGAGCC (SEQ ID NO: 38), GGGTCTAAATTTGGATTGATTTATGC (SEQ ID NO: 39), GGCACCTTCAGTTTTGTACTATTGGCTTTGAATC (SEQ ID NO: 40), GCACCTTCAGTTTTGTACTATTGGCTTTGAATCAA (SEQ ID NO: 41), CGTCATCTGATTGTAAGCACAATATGAGTTGTGCC (SEQ ID NO: 42), CCTCCAAACCACGTCATCTGATTGTAAGCACAAT (SEQ ID NO: 43), ACATTTCTGGGCTCTCTGACCCC (SEQ ID NO: 44), AACCCCTAGAAAACGTA (SEQ ID NO: 45), TTTAGAAAAAATCAAAGACCATGATTTATGAAAC (SEQ ID NO: 46), TCGTGATGAAACTTAAATATATATTCTTTGTC (SEQ ID NO: 47), GTGTGATTCATGTAGGACTTC (SEQ ID NO: 48), GGGCTCTACAGCAGTCGTGATGAAACTTAAATAT (SEQ ID NO: 49), GCCTTAAAATTTAAAAAGCAGGGCCCAAAGCTTA (SEQ ID NO: 50), GCCTTAAAATTTAAAAAGCAGGGCCCAAAGC (SEQ ID NO: 51), GCACCTTCAGTTTTGTACTATTGGCTTTGAATCA (SEQ ID NO: 52), GAAAGGGAGTATTATTATAGTATAC (SEQ ID NO: 53), CTCACACATTTGAGGCAGTGG (SEQ ID NO: 54), ATAGTACTTGTAATTTCTTTCTGCTTAGAATC (SEQ ID NO: 55), AGGTAAAATCAAATATAACTACAGC (SEQ ID NO: 56), AGATTTCCTTGTAAAATGTG (SEQ ID NO: 57), ACCACGTCATCTGATTGTAAGC (SEQ ID NO: 58), ACAGGTAAAGCCTATGATGTGTGT (SEQ ID NO: 59), AATATGAGTTGTGCCCCAATGCTCG (SEQ ID NO: 60), AACTAATGTGACATAATTTCCAGTGA (SEQ ID NO: 61), TGGAAAGGGAGTATTATTATAGTATACAACACTG (SEQ ID NO: 62), TGACTTGTTGATGTGAAATCAGACAC (SEQ ID NO: 63), TACAGCATAAGAGAATCGAGAAATTTGATAGAGG (SEQ ID NO: 64), GTTATAACATGATAGGTGCTGAATT (SEQ ID NO: 65), GTAAATCTAATAGTACTTGTAATTTCTTTCTGCT (SEQ ID NO: 66), GGTAAAGCCTATGATGTGTGTCATTTTAAAGTGTCG (SEQ ID NO: 67), GGTAAAGCCTATGATGTGTGTCATTTTAAAGTGT (SEQ ID NO: 68), GGGCTCTACAGCAGTCGTGATGAAACTTAAATATATATTCT (SEQ ID NO: 69), GCGAGAGAGGATGTATACTTTTCAAGAGAGATGA (SEQ ID NO: 70), CTAGTGGAACAGTCAGTTTAAC (SEQ ID NO: 71), ATGGTAGAGAAACACACATGC (SEQ ID NO: 72), ATGCTTTAATTTTGCACATTCGTACTATAGGGAGC (SEQ ID NO: 73), ATCAATTCAAAACCCCTAGAAAACGTATACAG (SEQ ID NO: 74), ATAAGATTTCTAACATTTCTGGGCTCTCTGACCCCT (SEQ ID NO: 75), AGAAACACACATGCCTT (SEQ ID NO: 76), ACCACGTCATCTGATTGTAAGCACAATATGAGTTC (SEQ ID NO: 77), AAGCCTATGATGTGTGTCATTTTAAAGTGTCGGA (SEQ ID NO: 78), AAATCTAGTGGAACAGTCAGTTTAACTTTTTAACAGA (SEQ ID NO: 79), AAACCACGTCATCTGATTGTAAGC (SEQ ID NO: 80), or can include fragments of these sequences, or can include these sequences as substrings.

In some embodiments, a short RNA sequence can have an overlapping region with a pyknon. Pyknons are repeated DNA sequences that appear at least 30 times or more in the intergenic and/or intronic sequences of a genome and have at least one additional instance in the exon (untranslated or protein-coding region) of a protein-coding gene.

In some embodiments of the methods or assays described herein, detection of the presence or absence of the short RNA sequence(s) can include measuring an expression level of the short RNA sequence(s) in the biological sample. The expression level of the short RNA sequence(s) can be detected by any methods known in the art, including, but not limited to, sequencing, next-generation sequencing (e.g., deep sequencing), polymerase chain reaction (PCR), and real-time quantitative PCR, northern blot, microarray, in situ hybridization, serial analysis of gene expression (SAGE), cap analysis gene expression (CAGE), massively parallel signature sequencing (MPSS), direct multiplexed measurements of the type employed in the Nanostring platform, and any combinations thereof. In such embodiments, the methods or assays can further comprise comparing with a reference sample the determined expression level of the short RNA sequence(s) in the biological sample. When there is a discrepancy in the expression level or amount of at least one short RNA sequence between the biological sample and the reference sample, the discrepancy can be indicative of the cell or the tissue in a state different from the reference sample. For example, in some embodiments, the discrepancy can be indicative of a subject either having, or being at risk of developing, or being at a given stage of a disease or disorder, e.g., a condition afflicting the tissue. In alternative embodiments, the discrepancy can be indicative of a subject lacking of a disease or disorder to be evaluated.

In some embodiments of the methods or assays described herein, detection of the presence or absence of the short RNA sequence(s) can include identifying an originating location of the short RNA sequence(s) from the exon or from the non-coding transcript. For example, the short RNA sequence(s) can be mapped to a reference genome to determine its location along one or more exons of a protein-coding gene, or along one or more segments of a non-coding transcript, using any art-recognized bioinformatics alignment tools such as short-read alignment tools. In such embodiments, the methods or assays can further comprise comparing with a reference sample the originating location of the short RNA sequence(s) or a profile of the short RNA sequences, wherein a discrepancy in the originating location or profile of the short RNA sequence(s) from the reference sample is indicative of the cell or the tissue in a state different from the reference sample. For example, in some embodiments, the discrepancy can be indicative of a subject either having, or being at risk of developing, or being at a given stage of a disease or disorder, e.g., a condition afflicting the tissue. In alternative embodiments, the discrepancy can be indicative of a subject lacking of a disease or disorder to be evaluated.

The reference sample used in the methods, assays and systems described herein can be a sample derived from the same type of cell or tissue with a known condition. For example, the reference sample can represent a normal condition of a cell or tissue to be detected. The normal reference sample can be obtained from the test subject or a different subject. Alternatively, the reference sample can represent a recognizable stage of a possibly abnormal condition of a cell or a tissue to be detected.

A biological sample for evaluation in the methods, assays, and systems described herein can include one or more cells derived from any tissue or fluid in a subject. In one embodiment, the biological sample can be a tissue suspected of being at risk of, or being afflicted with a given stage of a disease or a disorder. Non-limiting examples of sample origins can include, but are not limited to, breast, pancreas, blood, prostate, colon, lung, skin, brain, ovary, kidney, oral cavity, throat, cerebrospinal fluid, and liver.

Different embodiments of the methods, assays and systems described herein can be used for diagnosis and/or prognosis of a disease or disorder (including a given stage of a disease or disorder) in a subject, e.g., a disease or disorder afflicting a certain tissue in a subject. For example, the disease or disorder to be diagnosed and/or prognosed in a subject can be associated with breast, pancreas, blood, prostate, colon, lung, skin, brain, ovary, kidney, oral cavity, throat, cerebrospinal fluid, liver, and any combination thereof. In some embodiments, the disease or disorder to be diagnosed and/or prognosed with the methods, assays or systems described herein can be a blood disorder, e.g., associated with diseased or abnormal platelets. In other embodiments, the disease or disorder to be diagnosed and/or prognosed with the methods, assays or systems described herein can be any cancer, e.g., but not limited to breast cancer and pancreatic cancer.

In some embodiments, the methods, assays and systems described herein for determining a cellular state or tissue state of a biological sample can be used for determining in a subject a given stage of cancer. Accordingly, methods, systems and assays for determining in a subject a given stage of cancer are also provided herein. For example, such methods and assays can comprise detecting in a biological sample (e.g., a biopsy) the presence or absence of a short RNA sequence originating from an exon of at least one protein-coding gene, and/or from a segment of at least non-coding transcript.

In some embodiments, the cancer to be diagnosed and/or prognosed can be breast carcinoma. In such embodiments, the methods or assays described herein can be used to distinguish a cancerous breast tissue from a normal breast tissue, or identify a given state of a breast carcinoma, e.g., ductal carcinoma in situ (DCIS), lobular carcinoma in situ or invasive carcinoma (INV).

To distinguish a cancerous breast tissue from a normal breast tissue and/or to determine whether a breast tissue is DCIS or not, in some embodiments, the methods or assays described herein can comprise detecting the presence or absence of a short RNA sequence originating from one or more exons of a protein-coding gene, and/or one or more segments of a non-coding transcript. Examples of protein-coding genes whose exons are pertinent for DCIS can include, without limitations, ABCC11, ACTB, ACTG1, AHCY, AHNAK, ANKHD1, APP, ARF1, ASAH1, ATP1A1, ATP1B1, ATP6V0E1, AZGP1, B2M, B4GALT1, BAZ2A, BST2, BTG2, C1orf43, C3orf1, CALM2, CALR, CANX, CCNI, CD151, CD164, CD44, CD46, CD59, CD74, CEACAM6, CIRBP, CLIC6, COL1A2, COL6A1, COL6A3, COMMD3, COX7A2, CSDE1, CSRP1, CST3, CTNND1, CTSB, CXCL13, CYBRD1, DCN, DDX17, DDX5, DSP, DUSP4, EEF2, EFHD1, EHF, EIF4EBP3, EIF4G2, ELOVL5, ERBB2, ERBB3, ESR1, FASN, FAT1, FLNB, FMOD, FN1, FOXA1, FTL, GAPDH, GATA3, GDI2, GJA1, GLUL, HDLBP, HIST1H1B, HIST1H2AC, HIST1H3D, HIST1H4H, HNRNPF, HSP90AB1, IFI6, IGFBP4, IGHG4, ITGB4, JUP, KIAA0100, KIAA1522, LAPTM4A, LPHN1, LRBA, LRP2, MAGED2, MDH1, MED13L, MKNK2, MLL5, MLPH, MT-CO2, MUC1, MYB, MYH9, MYL6, NCL, NDUFA2, NET1, NF1, NME1, NUCKS1, P4HB, PACS2, PCBP2, PDCD4, PDIA3, PDLIM1, PDXDC1, PEG10, PFN1, PGR, PI15, PNRC1, PPDPF, PSMD5, PTPRF, QDPR, RARG, RBM39, RHOA, RHOB, RNF41, RPL15, S100A16, SEC11A, SERPINA1, SERPINA3, SFRP2, SH3BGRL, SIAH2, SLC25A6, SLC26A2, SLC38A1, SLC39A6, SLC7A2, SMG5, SREBF2, SRRM2, SSR2, STEAP1, STOM, TAGLN2, TAT, TFF3, TGOLN2, THAP4, TMBIM6, TMC5, TMED2, TMED5, TMEM59, TMEM66, TOB1, TPT1, TRPS1, TSPAN1, TTC39A, TUFM, TXNIP, UBN1, UBXN4, UFC1, UGDH, UNC13B, VIM, WAPAL, WIPI1, WNK1, XBP1, ZBTB7B, and any combinations thereof.

To distinguish a cancerous breast tissue from a normal breast tissue and/or to determine whether a breast tissue is INV or not, in some embodiments, the methods or assays described herein can comprise detecting the presence or absence of a short RNA sequence originating from one or more exons of a protein-coding gene, and/or one or more segments of a non-coding transcript. Examples of protein-coding genes whose exons are pertinent for INV can include, without limitations, ABCC11, ACTB, ACTG1, ADAR, AFF3, AHCY, AHNAK, ANKHD1, APP, ARF1, ARHGDIB, ASAH1, ATP1A1, ATP1B1, ATP6V0E1, AZGP1, B2M, B4GALT1, BAZ2A, BST2, BTG2, C1orf43, C5orf45, CALM2, CALR, CANX, CCNI, CD151, CD164, CD44, CD46, CD59, CD74, CD81, CEACAM6, CELSR1, CELSR2, CEP350, CILP, CIRBP, CLDN4, CLIC6, COL1A2, COL3A1, COL6A3, COMMD3, COX7A2, CSDE1, CSRP1, CTNNA1, CTNNB1, CTSD, CXCL13, CYBRD1, DBI, DCN, DDX17, DDX5, DSP, DUSP4, EEF2, EFHD1, EHF, EIF4EBP3, EIF4G2, ELF3, ELOVL5, EPRS, ERBB2, ERBB3, ESR1, FASN, FHL2, FLNB, FMOD, FOXA1, FTH1, GAPDH, GATA3, GDI2, GJA1, GLUL, GNAS, GNB2L1, GSTK1, HDLBP, HIST1H1C, HIST1H1D, HIST1H1E, HIST1H2AC, HIST1H2AE, HIST1H2BC, HIST1H2BD, HIST1H3D, HIST1H4B, HIST1H4D, HIST1H4H, HIST2H2AB, HIST2H2AC, HIST4H4, HNRNPF, HSP90AA1, HSP90AB1, IFI6, IGFBP4, IGHG1, IGHG4, IGKC, JTB, JUP, KIAA0100, KIAA1522, KRT19, LAPTM4A, LMNA, LONP2, LPHN1, LRBA, MAGED2, MCL1, MDH1, MED13L, MGP, MKNK2, MLL5, MLPH, MPZL1, MT-CO2, MT-CYB, MUC1, MYB, MYH9, MYST3, NCL, NDUFA2, NDUFB5, NET1, NF1, NFIB, NME1, NUCKS1, OAZ1, P4HB, PACS2, PCBP2, PDCD4, PDIA3, PDLIM1, PDXDC1, PEG10, PFN1, PGR, PHB2, PI15, PNRC1, PPDPF, PRICKLE4, PSAP, PTPRF, QDPR, RARG, RBM39, RHOA, RHOB, RNF41, RPL13AP20, RPL15, RPL17, RPL4, RPLP2, RPS2, S100A11, S100A14, S100A16, S100A9, SAT1, SEMA3C, SERPINA1, SERPINA3, SF3B1, SGK3, SH3BGRL, SIAH2, SLC25A3, SLC25A6, SLC26A2, SLC38A1, SLC39A6, SLC7A2, SMG5, SPARC, SPTBN1, SREBF2, SRRM2, SSR2, STEAP1, STOM, TAGLN2, TAT, TBC1D16, TFF3, TGOLN2, THAP4, TM9SF2, TMBIM6, TMC5, TMED2, TMEM59, TMEM66, TOB1, TOMM6, TPT1, TRPS1, TSPAN1, TTC39A, TUFM, TXNIP, UCK2, UFC1, UGDH, UNC13B, WIPI1, WNK1, XBP1, ZBTB7B, ZNF207, and any combinations thereof.

In some embodiments, the cancer to be diagnosed or prognosed can be pancreatic cancer. In such embodiments, the methods or assays described herein can be used to distinguish a cancerous pancreas tissue from a normal pancreas tissue, or to identify a given state of a pancreatic cancer, e.g., early-stage pancreatic cancer or late-stage pancreatic cancer.

To distinguish a cancerous pancreas tissue from a normal pancreas tissue and/or to determine whether a pancreas tissue has or is at risk of having early-stage pancreas cancer, in some embodiments, the methods or assays described herein can comprise detecting the presence or absence of a short RNA sequence originating from one or more exons of a protein-coding gene, and/or one or more segments of a non-coding transcript. Examples of protein-coding genes whose exons are pertinent for early-stage pancreatic cancer can include, without limitations, ACTG1, ALB, AMY2B, C7, CEL, CELA3A, CLPS, COL3A1, CPA1, CPA2, CPB1, CTRB1, CTRB2, CUZD1, EEF2, GANAB, GATM, GP2, HDLBP, KHDRBS1, KLK1, KRT7, OLFM4, P4HB, PLA2G1B, PPDPF, PRSS1, PRSS3, REG1A, REG1B, REG3A, RNASE1, RPL8, SPINK1, SYCN, UNC13B, and any combinations thereof.

To distinguish a cancerous pancreas tissue from a normal pancreas tissue and/or to determine whether the pancreas tissue has or is at risk of having late-stage pancreas cancer, in some embodiments, the methods or assays described herein can comprise detecting the presence or absence of a short RNA sequence originating from one or more exons of a protein-coding gene, and/or one or more segments of a non-coding transcript. Examples of protein-coding genes whose exons are pertinent for late-stage pancreatic cancer can include, without limitations, ACTB, ANXA2, ANXA5, APOE, ATP6VOC, C1QA, C1QB, C1QC, CIS, CALR, CCNI, CD14, CD44, CD59, CD68, COL1A2, COL6A3, CTSB, CTSC, EEF2, F13A1, FLNA, FN1, GLUL, GPNMB, GPX1, HIST1H2BD, IGFBP4, IGHM, IGKC, ISG15, LAMB3, LAPTM5, LGALS3BP, METTL7A, MMP11, MMP14, MT-CO2, MT-CYB, MYH9, OAZ1, P4HB, PLEC, PSAP, RNASE1, RPN1, SAT1, SERPINA1, SERPING1, SLC40A1, SLCO2B1, SPP1, SRGN, TGM2, TGOLN2, TIMP2, TXNIP, VSIG4, ZYX, and any combinations thereof.

In some embodiments of any aspects described herein, a short RNA sequence can be originated from one or more exons of a protein-coding gene, the protein encoded by which is not present or the expression of which is not detectable in a biological sample. For example, even a given protein may not be present or detectable in a biological sample, short RNAs that originate from one or more exons that would normally make the mRNA of the protein can be present and/or detectable, and thus can be used as biomarker for diagnostic or prognostic methods and/or systems described herein.

For a subject who is determined to have, or is at risk of developing, or is at a given stage of the disease or disorder, the subject can be administered or prescribed with a specific treatment. For example, in some embodiments where the subject is diagnosed with cancer (e.g., breast carcinoma or pancreatic carcinoma) or progression thereof, the method can further comprise administering or prescribing the subject a treatment, e.g., chemotherapy, radiation therapy, surgery, engineered transcripts that can “sponge” various combinations of the short RNAs described herein, or any combinations thereof.

Another aspect provided herein relates to systems for analyzing a biological sample, e.g., to determine a given state of a cell or a tissue, and/or to diagnose and/or prognose a disease or disorder, or a given state of a disease or disorder in a subject. In one embodiment, the system comprises: (a) a determination module configured to receive a biological sample and to determine sequence information and, optionally quantity estimate information, wherein the sequence information comprises a sequence of a short RNA molecule originating from an exon of at least one protein-coding gene, and/or a segment of at least one non-coding transcript; and wherein the quantity estimate information comprises at least an estimate of the abundance of said sequence, with said abundance optionally scaled with regard to the abundance of a reference molecule; (b) a storage device configured to store sequence information and optionally the quantity estimate information from the determination module; (c) a comparison module adapted to compare the sequence information and optionally the quantity estimate information stored on the storage device with reference data, and to provide a comparison result, wherein the comparison result identifies the presence or absence of the short RNA molecule, and optionally how its quantity estimate is related to the reference data, wherein a discrepancy in a quantity estimate level and/or in an originating location of the short RNA molecule from the reference data is indicative of the biological sample having an increased likelihood of having, or being at a cellular or tissue state different from a state represented by the reference data; and (d) a display module for displaying a content based in part on the comparison result for the user, wherein the content is a signal indicative of a subject having, or being at risk of developing, or being at a given stage of a disease or disorder, or a signal indicative of lacking a disease or disorder.

A computer-readable physical medium for determination of a given state of a cell or a tissue, including diagnosis and/or prognosis of a disease or disorder, or a state of a disease or disorder in a subject, is also provided herein. The computer-readable physical medium having computer readable instructions recorded thereon to define software modules includes a comparison module and a display module for implementing a method on a computer, wherein the method comprises: (a) comparing with the comparison module the data stored on a storage device with reference data to provide a comparison result, wherein the comparison result captures the presence or absence of the short RNA molecule and/or the difference between its quantity estimate and the reference data, wherein a discrepancy in a quantity estimate level or in an originating location of the short RNA molecule from the reference data is indicative of a biological sample having an increased likelihood of having, or being at a cellular or tissue state different from a state represented by the reference data; and (b) a display module for displaying a content based in part on the comparison result for the user, wherein the content is a signal indicative of a subject having, or being at risk of developing, or being at a given stage of a disease or disorder, or a signal indicative of lack of a disease or disorder.

Without wishing to be bound, in some embodiments of any aspects described herein, the methods, assays and systems described herein can be used to identify an origin and/or type of a cell or a tissue (e.g., to identify whether a cell or tissue is derived from breast, pancreas, liver, lung or other tissue of a body). Additionally, the methods, assays and systems described herein can be used to distinguish an origin and/or type of a first tissue from a second tissue. For example, such method can comprise detecting in a first biological sample the presence or absence of a short RNA sequence originating from an exon of at least one protein-coding gene, and/or a segment of at least one non-coding transcript, wherein a difference in an expression level of the short RNA sequence between the first and the second biological sample is indicative of the first tissue having an origin and/or type different from that of the second tissue. In some embodiments, the method can further comprise detecting in a second biological sample the presence or absence of the short RNA sequence.

### BRIEF DESCRIPTION OF THE FIGURES

FIG. 1 shows the locations and amount of the sequenced short RNA molecules that originate from the last exon of gene ELOVL5 from four breast samples, including 2 normal (Breast_{—}1N1 and Breast_{—}2N2), 1 ductal in situ carcinoma (Breast_{—}1D1) and 1 invasive carcinoma (Breast_{—}2D2).

FIG. 2 shows the locations and amount of the sequenced short RNA molecules that originate from the last two exons of gene ESR1 from four breast samples, including 2 normal (Breast_{—}1N1 and Breast_{—}2N2), 1 ductal in situ carcinoma (Breast_{—}1D1) and 1 invasive carcinoma (Breast_{—}2D2).

FIG. 3 shows the locations and amount of the sequenced short RNA molecules that originate from a number of exons of gene SRRM2 from four breast samples, including 2 normal (Breast_{—}1N1 and Breast_{—}2N2), 1 ductal in situ carcinoma (Breast_{—}1D1) and 1 invasive carcinoma (Breast_{—}2D2).

FIG. 4 shows the locations and amount of the sequenced short RNA molecules that originate from an exon of gene AHNAK from four breast samples, including 2 normal (Breast_{—}1N1 and Breast_{—}2N2), 1 ductal in situ carcinoma (Breast_{—}1D1) and 1 invasive carcinoma (Breast_{—}2D2).

FIG. 5 shows the locations and amount of the sequenced short RNA molecules that originate from a set of exons of gene CEL from four pancreatic samples, including 2 normal (Pancreas_{—}1N1 and Pancreas_{—}2N2), 1 early stage (Pancreas_{—}1D1) and 1 late stage (Pancreas_{—}2D2).

FIG. 6 shows the locations and amount of the sequenced short RNA molecules that originate from a set of exons of gene GP2 from four pancreatic samples, including 2 normal (Pancreas_{—}1N1 and Pancreas_{—}2N2), 1 early stage (Pancreas_{—}1D1) and 1 late stage (Pancreas_{—}2D2).

In FIGS. 1-6, the Y-axis is logarithmic (base 2). The height at a given location of the X-axis indicates the log2 of the number of sequenced reads that cover this location when mapped on the genome. Since the X-axis in each case spans a sizeable genomic region, the overlapping reads at a given location are ‘condensed’ and lead to the apparent arrangement of “spikes.”

FIG. 7 is a block diagram showing an example of a system for determining a state of a cell or a tissue and/or for diagnosing or prognosing a disease or disorder, or a stage of a disease or disorder in a subject.

FIG. 8 is a block diagram showing exemplary instructions on a computer readable medium for determining a state of a cell or a tissue and/or for diagnosing or prognosing a disease or disorder, or a stage of a disease or disorder in a subject.

### DETAILED DESCRIPTION

While there are imaging tools (e.g., X-ray, MRI, CT scans) and/or laboratory tests (e.g., biomarker assay) for diagnosing a disease or disorder, these technologies are not sensitive or reliable enough for differentiating different individual stages of a disease or disorder. Thus, pathology still remains as the gold standard for diagnosis of various diseases such as cancer or disorders, and/or determination of a given stage of the disease, e.g., cancer, or disorder. However, proper technical skills for processing a biopsy sample and experienced pathologists are critical, or difficult interpretation problems can arise. Thus, there is still a strong need to develop methods that are more definitive and reliable for diagnosing or determining a given state of a disease such as cancer or disorder.

In accordance with different aspects described herein, short RNA molecules originating from one or more exons of a protein-coding gene have been discovered for association with distinguishing or identifying a specific cell or tissue or a specific stage or condition of a cell or a tissue. It is generally known that exons of a protein-coding region are used to compose a transcript, the mRNA, which is translated by a ribosome into an amino acid sequence or protein. Thus, it is a surprising discovery of short RNA molecules originating from one or more exons of a protein-coding gene in cells from a tissue (e.g., somatic tissue) such as breast or pancreas. More importantly, the presence/absence and/or amount of the short RNA molecules originating from one or more exons of a protein-coding gene varies with a state or condition of a cell or tissue. For example, while there are only a few short RNA molecules originating from one or more exons of protein-coding genes such as ELOVL5, ESR1, SRRM2 and AHNAK in a normal breast tissue sample, there are numerous short RNA molecules produced from the exon(s) of those genes in a DCIS breast tissue sample. Interestingly, in the pancreatic tissue samples, significantly more short RNA molecules originating from exons of a protein-coding gene such as CEL or GP2 are detected in normal tissues while little or no short RNA molecules are detected in the cancerous tissues. Thus, normal tissues (e.g., breast tissue and pancreatic tissues) can be differentiated from cancerous tissues (e.g., breast carcinoma and pancreatic carcinoma) based on a difference in expression levels or profiling or the location of origin of short RNA molecules detected in the normal and cancerous tissues. Furthermore, a given state or condition of a disease (e.g., cancer) or disorder can be determined by detecting the presence/absence and/or location and/or amount of the short RNA molecules originating from one or more exons of a protein-coding gene. For example, there are significantly more short RNA molecules generated from one or more exons of a protein-coding gene (e.g., ELOVL5, ESR1, SRRM2 and AHNAK) in ductal carcinoma in situ (DCIS) breast tissues than in normal breast tissues. Accordingly, provided herein generally relates to methods, assays and systems for determining a given state of a cell and/or a tissue. In some embodiments, the methods, assays, systems can be used to determine the origin (e.g., identity) of a cell and/or a tissue. Methods for diagnosing a disease or disorder, and/or prognosing a given stage and/or progression of a disease or disorder are also provided herein.

### Methods and Assays for Determining a Given State of a Cell or a Tissue

One aspect described herein provides methods and assays for determining a specific state or condition of a cell or a tissue. As the cell or tissue can be derived from a biological sample of a subject suspected of being at risk of or having a given stage of a disease or disorder, e.g., a condition afflicting a tissue, methods and assays for determining whether a subject has or is at risk of developing, or is at a given stage of a disease or disorder, e.g., a condition afflicting a tissue, are also provided herein. The methods or assays of any aspects described herein comprise detecting in a biological sample the presence or absence of a short RNA sequence corresponding to at least part of an exon of at least one protein-coding gene or to a segment of a non-coding transcript. In various embodiments, at least one or more short RNA sequences originate from at least part of an exon (including one or more exons) of at least one protein-coding gene and/or from at least one segment (including one or more segments) of one or more non-coding transcripts.

In some embodiments of any aspects described herein, the method or assay can comprise detecting in the biological sample the presence or absence of one short RNA sequence corresponding to at least part of an exon of at least one or more protein-coding genes and/or to a segment of at least one or more non-coding transcripts.

In some embodiments of any aspects described herein, the method or assay can comprise detecting in the biological sample the presence or absence of a plurality of short RNA sequences corresponding to at least part of an exon of at least one or more protein-coding genes, and/or to a segment of at least one or more non-coding transcripts. In some embodiments, the plurality of the short RNA sequences can originate from more than one locations of an exon of at least one or more protein-coding gene, and/or from more than one locations of a segment of at least one or more non-coding transcripts. In other embodiments, the plurality of the short RNA sequences can originate from one or more locations of a plurality of exons (e.g., 2, 3, 4, 5, 6, 7, 8, 9, 10, or more exons) of at least one or more protein-coding genes. In some embodiments, the plurality of the short RNA sequences can originate from one or more locations of a plurality of segments (e.g., 2, 3, 4, 5, 6, 7, 8, 9, 10, or more segments) of at least one or more non-coding transcripts. As used herein, the phrase “a plurality of short RNA sequences” refers to at least two or more distinct short RNA sequences. In some embodiments, at least two or more short RNA sequences can differ in sequence composition that can possibly originate from overlapping genomic locations. In some embodiments, the phrase “a plurality of short RNA sequences” includes at least about 2, at least about 3, at least about 4, at least about 5, at least about 6, at least about 7, at least about 8, at least about 9, at least about 10, at least about 15, at least about 25, at least about 50, at least about 100, at least about 250, at least about 500, at least about 750, at least about 1000, at least about 2500, at least about 5000, at least about 10,000 or more, distinct short RNA sequences. By the phrase “distinct short RNA sequences” used herein is meant each short RNA sequence having at least one nucleotide (including at least 2, at least 3, at least 4, at least 5, at least 6, at least 7, at least 8, at least 9, at least 10, at least 15, at least 20, at least 30, at least 40, at least 50 or more nucleotides) different from each other. In one embodiment, distinct short RNA sequences are short RNA sequences each corresponding to a different, non-overlapping exonic region of a protein-coding gene and/or to a different, non-overlapping segment of a non-coding transcript.

In some embodiments, the methods or assays of any aspects described herein can comprise detecting in a biological sample the presence or absence of one or a plurality of short RNA sequences corresponding to an exon of more than one protein-coding genes, e.g., including 2, 3, 4, 5, 6, 7, 8, 9, 10, 25, 50, 75, 100, 250, 500, 750, 1000, 2500, 5000, 7500, 10000 or more protein-coding genes.

In some embodiments, the methods or assays of any aspects described herein can comprise detecting in a biological sample the presence or absence of one or a plurality of short RNA sequences corresponding to a segment of more than one non-coding transcripts, e.g., including 2, 3, 4, 5, 6, 7, 8, 9, 10, 25, 50, 75, 100, 250, 500, 750, 1000, 2500, 5000, 7500, 10000 or more non-coding transcripts.

While detecting the presence or absence of the short RNA sequence(s), in some embodiments, an amount of a short RNA sequence in the biological sample can be measured or quantified by any known RNA detection methods. By way of example only, the short RNA sequence(s) in a biological sample can be detected or read by a sequencing method (including Sanger sequencing, next-generation sequencing or deep sequencing, direct multiplexing, and any art-recognized sequencing method) and a read count of each short RNA sequence can be generated to determine its amount present in the biological sample. Alternatively, where the short RNA sequence(s) in a biological sample are determined by PCR-based methods (e.g., real-time PCR), the amount of the short RNA sequence(s) present in the biological sample can be represented by a Cₜ number, which can be compared to that of a reference sample. As a person having ordinary skill in the art would appreciate, a larger Cₜ number generally indicates a lower amount of a nucleic acid sequence present in a sample. In some embodiments, the quantitative amount of the short RNA sequence(s) detected by PCR-based methods (e.g., real-time PCR) can also be determined from a calibration curve generated with known amounts of a nucleic acid sequence.

In some embodiments, rather than comparing amounts of individual short RNA sequences present in the biological sample with those in a reference sample, the total amount of short RNA sequences originating from one exon of a protein-coding gene and/or from a segment of a non-coding transcript, present in the biological sample, can be compared to that in a reference sample. In some embodiments, the total amount of short RNA sequences originating from two exons or more (including 2, 3, 4, 5, 6, 7, 8, 9, 10 or more exons) of a protein-coding gene, and/or from two or more segments (including 2, 3, 4, 5, 6, 7, 8, 9, 10 or more segments) of a non-coding transcript, present in the biological sample, can be compared to that in a reference sample. In some embodiments, the total amount of all short RNA sequences originating from all exons of all protein coding loci that are present in the biological sample can be compared to that in a reference sample. In some embodiments, the total amount of all short RNA sequences originating from all segments of all non-coding transcripts that are present in the biological sample can be compared to that in a reference sample.

As the amount of the short RNA sequence(s) is determined in the biological sample, in some embodiments, the methods or assays described herein can further comprise comparing with a reference sample the amount of one or more short RNA sequences in the biological sample. When there is a difference (e.g., at least about 10% difference or higher) or a statistically significant difference in an amount of at least one or more (e.g., at least 2 or more) or in the total amount of short RNA sequences between the biological sample and the reference sample, the difference or significant difference can be indicative of the cell or the tissue in a state different from the reference sample. If the cell or the tissue is derived from a biological sample of a subject, the results of the comparison can be used for diagnosing or prognosing a disease or disorder, or a state of a disease or disorder. Depending on the choice of a reference sample, in some embodiments, the difference or significant difference can be indicative of a subject either having, or being at risk of developing, or being at a given stage of a disease or disorder, e.g., a condition afflicting the tissue; while in other embodiments, the difference or significant difference can be indicative of a subject lacking of a disease or disorder, e.g., a condition afflicting the tissue.

The threshold level selected to distinguish a given state of a cell or tissue from another, and/or to determine if a subject has, or is at risk of developing, or is at a given stage of a condition afflicting a tissue of interest can be determined experimentally. For example, by comparing the expressions and/or profiles of one or more short RNA molecules detected in a number of references samples of known conditions in a specific tissue (e.g., a normal breast sample vs. a DCIS or INV breast sample), e.g., by deep sequencing and/or quantitative RT-PCR, one of skill in the art can determine a threshold level for expressions of one or more short RNA molecules required to distinguish one condition from another (e.g., to distinguish a normal breast sample from a DCIS or INV breast sample). Similarly, by comparing the expressions and/or profiles of one or more short RNA molecules detected in a number of reference sample of the same condition in different tissues (e.g., a normal breast sample vs. a normal pancreas sample), e.g., by deep sequencing and/or quantitative RT-PCR, one of skill in the art can determine a threshold level for expressions of one or more short RNA molecules required to distinguish one tissue type from another (e.g., to distinguish a normal breast sample from a normal pancreas sample).

In some embodiments, the methods or assays described herein can further comprise identifying a genomic location along an exon from which the short RNA sequence originates. In some embodiments, the methods or assays described herein can further comprise identifying a genomic location along a non-coding transcript from which the short RNA sequence originates. For example, the short RNA sequence(s) can be mapped to a reference genome (e.g., a human genome) to determine its location along one or more exons of a protein-coding gene using any art-recognized bioinformatics mapping tools such as short-read mapping tools. Examples of short-read mapping tools can include, without limitations, Bfast, BioScope, Bowtie, Burrows-Wheeler Aligner (BWA), CLC bio, CloudBurst, Eland/Eland2, Exonerate, GenomeMapper, GnuMap, Karma, MAQ, MOM, Mosaik, MrFAST/MrsFAST, NovoAlign, PASS, PerM, RazerS, RMAP, SSAHA2, Segemehl, SeqMap, SHRiMP, Slider/SliderII, SOAP/SOAP2, Srprism, Stampy, vmatch, ZOOM and any art-recognized alignment tools that can be used to map short-read sequences to a reference genome. Alternatively, a more general purpose tool such as FAST, BLAST, or BLAT can be used. In one embodiment, Burrows-Wheeler Aligner (BWH) can be used to map short RNA sequences to a reference genome (e.g., a human genome). Additional details of using BWA for short-read alignment can be found, e.g., in Li and Durbin (2009) Bioinformatics 25 (14): 1754-1760. Additional details of using SHRiMP for short-read alignment can be found, e.g., in Matei, Dzamba et al. (2011) Bioinformatics 27(7): 1011-1012.

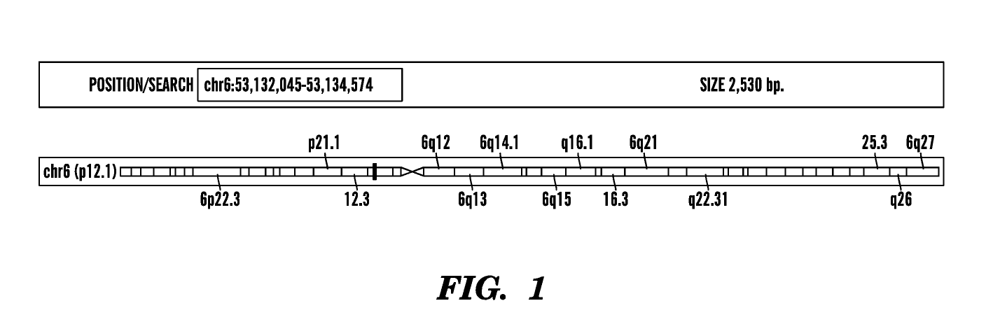

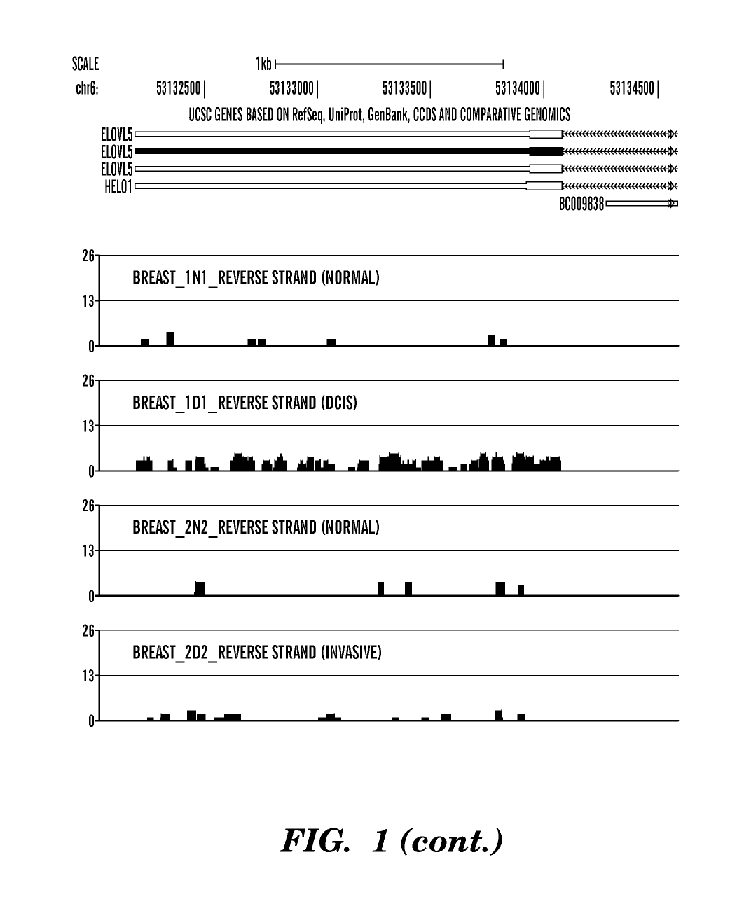

In some embodiments, the methods or assays described herein can further comprise profiling the short RNA sequences. Examples of profiling short RNA sequences are shown in Examples 2-7 and FIGS. 1-6, where the short RNA sequences have been sequenced and mapped to locations along one or more exons of a particular gene of a reference genome (e.g., human genome used for human tissue samples), as represented by red bars or blue bars in the figures. The height of the bar at a given genomic location represents the logarithm (base 2) of the number of overlapping sequenced reads that map to the location of interest.

Accordingly, in some embodiments, the method or assays can further comprise comparing with a reference sample the genomic location along one or more exons of a specific protein-coding gene from which the short RNA sequence(s) originate and/or a profile of the short RNA sequences for a specific protein-coding gene. In some embodiments, the method or assays can further comprise comparing with a reference sample the genomic location along one or more segments of a specific non-coding transcript from which the short RNA sequence(s) originate and/or a profile of the short RNA sequences for a specific non-coding transcript. For example, when short RNA sequences detected in the biological sample originate from genomic locations different from those detected in the reference sample (e.g., at least one or more short RNA sequences in the biological sample and reference sample are produced from different locations of an exon and/or from different exons), a shift in one or more genomic locations to which short RNA sequences are mapped can be observed between the biological sample and the reference sample. Additionally, a difference in the pattern of the short RNA sequence profile can be observed between the biological sample and the reference sample. A comparison of the two short RNA sequence profiles can be readily performed by a skilled artisan to determine if there is any significant difference between the two patterns. In some embodiments, a pattern recognition algorithm can be used to determine if there is any significant difference between the two patterns. The significant shift in the mapping locations and/or a significant difference in the profile pattern from a reference sample can be indicative of the cell or the tissue in a state different from the reference sample. In diagnostic and/or prognostic applications, depending on the reference sample, in some embodiments, the significant shift in the mapping locations and/or a difference in the profile pattern from a reference pattern can be indicative of a subject either having, or being at risk of developing, or being at a given stage of a disease or disorder, e.g., a condition afflicting the tissue; while in other embodiments, the significant shift in the mapping locations and/or a difference in the profile pattern from a reference pattern can be indicative of a subject lacking of a disease or disorder, e.g., a condition afflicting the tissue.

The reference sample used in the methods and assays described herein can be a sample derived from the same type of cell or tissue as the biological sample, and with a known condition. For example, the reference sample can represent a normal condition of a cell or tissue in a biological sample to be analyzed. Alternatively, the reference sample can represent a recognizable stage of an abnormal condition of a cell or a tissue in a biological sample to be analyzed. By way of example only, if a disease or disorder to be diagnosed or prognosed in a subject is breast cancer, a reference sample can include a normal breast tissue, a ductal carcinoma in situ breast tissue sample, an invasive ductal carcinoma tissue sample or subtype, an invasive lobular carcinoma tissue sample, a lobular carcinoma in situ tissue sample, and any combinations thereof.

In some embodiments, more than one reference samples can be used, wherein each of the reference samples can represent a different condition (e.g., normal and a given stage of a disease or disorder; or different stages of a disease or disorder). By way of example only, if a biological sample of a subject generates a similar or comparable short RNA sequence profile (e.g., in terms of amounts and/or locations of short RNA sequences) for a specific gene to that of a normal sample (as a reference sample), the subject can be considered normal with respect to that specific gene. While it may not be necessary, it can be desirable to detect short RNA sequences for other different genes in the biological sample and compare to a normal sample to determine if similar conclusions are obtained. Additionally or alternatively, the subject's short RNA sequence profile for the specific gene can be further compared to that of a diseased or abnormal sample. If the subject's short RNA sequence profile is significantly different from that of the diseased or abnormal sample, it can be indicative of the subject lacking the disease or disorder to be diagnosed.

For a subject who is determined to have, or is at risk of developing, or is at a given stage of the disease or disorder, the subject can be administered or prescribed with a specific treatment. For example, in some embodiments where the subject is diagnosed with cancer (e.g., breast carcinoma or pancreatic carcinoma) or progression thereof, the method can further comprise administering or prescribing the subject a treatment, e.g., chemotherapy, radiation therapy, surgery, engineered transcripts that can “sponge” various combinations of the short RNAs described herein, or any combinations thereof.

In some embodiments where the amount of the short RNAs can be viewed to represent a “causal event” for the disease or disorder, the amount of the short RNAs can be controlled in order to return their levels to what would be considered “normal” levels and thus alleviate the impact that can result from the changes in their amount. Examples of the techniques that can be used to control the amount of the short RNAs include, but are not limited to, antisensing or sponging (e.g., microRNA sponges as described in Ebert and Shape. “MicroRNA sponges: Progress and possibilities” RNA (2010) 16:2043-2050; and Ebert et al. “MicroRNA sponges: Competitive inhibitors of small RNAs in mammalian cells” Nat. Methods (2007) 4: 721-726), decoying (e.g., as described in Swami M. “Small RNAs: Pseudogenes act as microRNA decoys.” Nature Reviews Genetics (2010) 11: 530-531), overexpression, and/or any art-recognized techniques.

Without wishing to be bound, in some embodiments of any aspects described herein, the methods, assays and systems described herein can be used to identify an origin or provenance and/or type of a cell or a tissue (e.g., to identify whether a cell or tissue is derived from breast, pancreas, liver, lung or other tissue of a body by determining expressions of one or more short RNA molecules or a profile of the short RNA molecules determined in the cell or tissue, which can then be compared with one or more reference samples corresponding to known cell or tissue types). Additionally, the methods, assays and systems described herein can be used to distinguish an origin and/or type of a first tissue from a second tissue. For example, such method can comprise detecting in a first biological sample the presence or absence of a short RNA sequence originating from an exon of at least one protein-coding gene and/or a segment of at least one non-coding transcript, wherein a difference in an expression level of the short RNA sequence between the first and the second biological sample is indicative of the first tissue having an origin and/or type different from that of the second tissue. In some embodiments, the method can further comprise detecting in a second biological sample the presence or absence of the short RNA sequence. In some embodiments, the method can further comprise comparing the expression level of the short RNA molecules detected in the first and/or second biological sample to at least one reference sample of a known tissue type. By way of example only, the short RNA molecules originated from gene CEL's exons are abundant in a normal pancreas tissue sample but absent or undetectable in a normal breast tissue sample. Accordingly, one can determine an origin/type of an unknown tissue and/or distinguish between two different unknown tissues, e.g., pancreas from breast, by determining expression of one or more short RNA molecules, or profiles of short RNA molecules, originated from one or more protein-coding genes in the sample(s) of interest.

In some embodiments, the methods described herein can be used to determine a primary origin of an unknown tumor or cancer. Thus, in some embodiments, the methods described herein can be used to determine whether the tumor is a primary tumor or a secondary tumor (i.e., a metastasis). For example, a biopsy of an unknown tumor can be subjected to the methods or assays described herein to determine the tissue origin of the tumor, wherein if the tissue origin of the tumor is determined to be the same tissue type as from where the biopsy is collected, the tumor is diagnosed as a primary tumor, or if the tissue origin of the tumor is determined to be different from the type of the tissue from where the biopsy is collected, the tumor is diagnosed as a secondary tumor (i.e., a metastasis).

In some embodiments, the methods described herein can be used to identify an origin of a biological sample, e.g., by comparing the measured expression level of one or more short RNA sequences with the reference level of a reference sample. Thus, the methods described herein can be used to fingerprint a biological sample, e.g., whether it is a normal sample or a diseased sample (e.g., a cancerous sample).

### Diseases or Disorders Amenable to Diagnosis or Prognosis Using any Aspects Described Herein

Different embodiments of the methods, assays and systems described herein can be used for diagnosis and/or prognosis of a disease or disorder, and/or the state of the disease or disorder in a subject, e.g., a condition afflicting a certain tissue in a subject. For example, the disease or disorder in a subject can be associated with breast, pancreas, blood, prostate, colon, lung, skin, brain, ovary, kidney, oral cavity, throat, cerebrospinal fluid, liver, testis, or other tissues, and any combination thereof.

In some embodiments, a disorder amenable to diagnosis and/or prognosis using any aspects described herein can include a condition that is not terminal but can cause an interruption, disturbance, or cessation of a bodily function, system, or organ. Such examples of disorders can include, e.g., but not limited to, developmental disorders (e.g., autism), brain disorders (e.g., epilepsy), mental disorders (e.g., depression), endocrine disorders (e.g., diabetes), or skin disorders (e.g., skin inflammation).

In some embodiments, the disease or disorder amenable to diagnosis and prognosis using any aspects described herein can include a breast disease or disorder. Exemplary breast disease or disorder includes breast cancer.

In some embodiments, the disease or disorder amenable to diagnosis and prognosis using any aspects described herein can include a pancreatic disease or disorder. Nonlimiting examples of pancreatic diseases or disorders include acute pancreatitis, chronic pancreatitis, hereditary pancreatitis, pancreatic cancer (e.g., endocrine or exocrine tumors), etc., and any combinations thereof.

In some embodiments, the disease or disorder amenable to diagnosis and prognosis using any aspects described herein can include a blood disease or disorder. Examples of blood disease or disorder include, but are not limited to, platelet disorders, von Willebrand diseases, deep vein thrombosis, pulmonary embolism, sickle cell anemia, thalassemia, anemia, aplastic anemia, fanconi anemia, hemochromatosis, hemolytic anemia, hemophilia, idiopathic thrombocytopenic purpura, iron deficiency anemia, pernicious anemia, polycythemia vera, thrombocythemia and thrombocytosis, thrombocytopenia, and any combinations thereof.

In some embodiments, the disease or disorder amenable to diagnosis and prognosis using any aspects described herein can include a prostate disease or disorder. Non-limiting examples of a prostate disease or disorder can include prostatitis, prostatic hyperplasia, prostate cancer, and any combinations thereof.

In some embodiments, the disease or disorder amenable to diagnosis and prognosis using any aspects described herein can include a colon disease or disorder. Exemplary colon diseases or disorders can include, but are not limited to, colorectal cancer, colonic polyps, ulcerative colitis, diverticulitis, and any combinations thereof.

In some embodiments, the disease or disorder amenable to diagnosis and prognosis using any aspects described herein can include a lung disease or disorder. Examples of lung diseases or disorders can include, but are not limited to, asthma, chronic obstructive pulmonary disease, infections, e.g., influenza, pneumonia and tuberculosis, and lung cancer.

In some embodiments, the disease or disorder amenable to diagnosis and prognosis using any aspects described herein can include a skin disease or disorder, or a skin condition. An exemplary skin disease or disorder can include psoriasis or skin cancer.

In some embodiments, the disease or disorder amenable to diagnosis and prognosis using any aspects described herein can include a brain disease or disorder. Examples of brain diseases or disorders can include, but are not limited to, brain infections (e.g., meningitis, encephalitis, brain abscess), brain tumor, glioblastoma, stroke, ischemic stroke, multiple sclerosis (MS), vasculitis, and neurodegenerative or neurological disorders (e.g., Parkinson's disease, Huntington's disease, Pick's disease, amyotrophic lateral sclerosis (ALS), dementia, and Alzheimer's disease), and any combinations thereof.

In some embodiments, the disease or disorder amenable to diagnosis and prognosis using any aspects described herein can include a liver disease or disorder. Examples of liver diseases or disorders can include, but are not limited to, hepatitis, cirrhosis, liver cancer, billary cirrhosis, primary sclerosing cholangitis, Budd-Chiari syndrome, hemochromatosis, transthyretin-related hereditary amyloidosis, Gilbert's syndrome, and any combinations thereof.

In other embodiments, the disease or disorder amenable to diagnosis and prognosis using any aspects described herein can include cancer. Examples of cancers can include, but are not limited to, bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS associated leukemias and adult T-cell leukemia lymphoma; intraepithelial neoplasms including Bowen's disease and Paget's disease, liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilm's tumor.

In some embodiments, the methods, assays and systems described herein can be used for determining in a subject a given stage of cancer. The stage of a cancer generally describes the extent the cancer has progressed and/or spread. The stage usually takes into account the size of a tumor, how deeply the tumor has penetrated, whether the tumor has invaded adjacent organs, how many lymph nodes the tumor has metastasized to (if any), and whether the tumor has spread to distant organs. Staging of cancer is generally used to assess prognosis of cancer as a predictor of survival, and cancer treatment is primarily determined by staging. Thus, methods, systems and assays for determining in a subject a given stage of cancer are also provided herein. For example, such methods and assays can comprise detecting in a biological sample (e.g., a biopsy) the presence or absence of a short RNA sequence originating from an exon of at least one protein-coding gene.

In some embodiments, the cancer to be diagnosed or prognosed can be breast carcinoma. In such embodiments, the methods or assays described herein can be used to distinguish a cancerous breast tissue from a normal breast tissue, or identify a given stage of a cancerous breast tissue, e.g., ductal carcinoma in situ, lobular carcinoma in situ, invasive ductal carcinoma or a subtype, invasive lobular carcinoma, etc.

To distinguish a cancerous breast tissue from a normal breast tissue and/or to determine whether the cancerous breast tissue is DCIS or not, in some embodiments, the methods or assays described herein can comprise detecting the presence or absence of a short RNA sequence originating from one or more exons of at least one protein-coding gene, and/or from one or more segments of at least one non-coding transcript. Examples of protein-coding genes whose exons are pertinent for DCIS can include, without limitations, ABCC11, ACTB, ACTG1, AHCY, AHNAK, ANKHD1, APP, ARF1, ASAH1, ATP1A1, ATP1B1, ATP6V0E1, AZGP1, B2M, B4GALT1, BAZ2A, BST2, BTG2, C1orf43, C3orf1, CALM2, CALR, CANX, CCNI, CD151, CD164, CD44, CD46, CD59, CD74, CEACAM6, CIRBP, CLIC6, COL1A2, COL6A1, COL6A3, COMMD3, COX7A2, CSDE1, CSRP1, CST3, CTNND1, CTSB, CXCL13, CYBRD1, DCN, DDX17, DDX5, DSP, DUSP4, EEF2, EFHD1, EHF, EIF4EBP3, EIF4G2, ELOVL5, ERBB2, ERBB3, ESR1, FASN, FAT1, FLNB, FMOD, FN1, FOXA1, FTL, GAPDH, GATA3, GDI2, GJA1, GLUL, HDLBP, HIST1H1B, HIST1H2AC, HIST1H3D, HIST1H4H, HNRNPF, HSP90AB1, IFI6, IGFBP4, IGHG4, ITGB4, JUP, KIAA0100, KIAA1522, LAPTM4A, LPHN1, LRBA, LRP2, MAGED2, MDH1, MED13L, MKNK2, MLL5, MLPH, MT-CO2, MUC1, MYB, MYH9, MYL6, NCL, NDUFA2, NET1, NF1, NME1, NUCKS1, P4HB, PACS2, PCBP2, PDCD4, PDIA3, PDLIM1, PDXDC1, PEG10, PFN1, PGR, PI15, PNRC1, PPDPF, PSMD5, PTPRF, QDPR, RARG, RBM39, RHOA, RHOB, RNF41, RPL15, S100A16, SEC11A, SERPINA1, SERPINA3, SFRP2, SH3BGRL, SIAH2, SLC25A6, SLC26A2, SLC38A1, SLC39A6, SLC7A2, SMG5, SREBF2, SRRM2, SSR2, STEAP1, STOM, TAGLN2, TAT, TFF3, TGOLN2, THAP4, TMBIM6, TMC5, TMED2, TMED5, TMEM59, TMEM66, TOB1, TPT1, TRPS1, TSPAN1, TTC39A, TUFM, TXNIP, UBN1, UBXN4, UFC1, UGDH, UNC13B, VIM, WAPAL, WIPI1, WNK1, XBP1, ZBTB7B, and any combinations thereof.

To distinguish a cancerous breast tissue from a normal breast tissue and/or to determine whether the cancerous breast tissue is INV or not, in some embodiments, the methods or assays described herein can comprise detecting the presence or absence of a short RNA sequence originating from one or more exons of at least one protein-coding gene, and/or from one or more segments of at least one non-coding transcript. Examples of protein-coding genes whose exons are pertinent for INV can include, without limitations, ABCC11, ACTB, ACTG1, ADAR, AFF3, AHCY, AHNAK, ANKHD1, APP, ARF1, ARHGDIB, ASAH1, ATP1A1, ATP1B1, ATP6V0E1, AZGP1, B2M, B4GALT1, BAZ2A, BST2, BTG2, C1orf43, C5orf45, CALM2, CALR, CANX, CCNI, CD151, CD164, CD44, CD46, CD59, CD74, CD81, CEACAM6, CELSR1, CELSR2, CEP350, CILP, CIRBP, CLDN4, CLIC6, COL1A2, COL3A1, COL6A3, COMMD3, COX7A2, CSDE1, CSRP1, CTNNA1, CTNNB1, CTSD, CXCL13, CYBRD1, DBI, DCN, DDX17, DDX5, DSP, DUSP4, EEF2, EFHD1, EHF, EIF4EBP3, EIF4G2, ELF3, ELOVL5, EPRS, ERBB2, ERBB3, ESR1, FASN, FHL2, FLNB, FMOD, FOXA1, FTH1, GAPDH, GATA3, GDI2, GJA1, GLUL, GNAS, GNB2L1, GSTK1, HDLBP, HIST1H1C, HIST1H1D, HIST1H1E, HIST1H2AC, HIST1H2AE, HIST1H2BC, HIST1H2BD, HIST1H3D, HIST1H4B, HIST1H4D, HIST1H4H, HIST2H2AB, HIST2H2AC, HIST4H4, HNRNPF, HSP90AA1, HSP90AB1, IFI6, IGFBP4, IGHG1, IGHG4, IGKC, JTB, JUP, KIAA0100, KIAA1522, KRT19, LAPTM4A, LMNA, LONP2, LPHN1, LRBA, MAGED2, MCL1, MDH1, MED13L, MGP, MKNK2, MLL5, MLPH, MPZL1, MT-CO2, MT-CYB, MUC1, MYB, MYH9, MYST3, NCL, NDUFA2, NDUFB5, NET1, NF1, NFIB, NME1, NUCKS1, OAZ1, P4HB, PACS2, PCBP2, PDCD4, PDIA3, PDLIM1, PDXDC1, PEG10, PFN1, PGR, PHB2, PI15, PNRC1, PPDPF, PRICKLE4, PSAP, PTPRF, QDPR, RARG, RBM39, RHOA, RHOB, RNF41, RPL13AP20, RPL15, RPL17, RPL4, RPLP2, RPS2, S100A11, S100A14, S100A16, S100A9, SAT1, SEMA3C, SERPINA1, SERPINA3, SF3B1, SGK3, SH3BGRL, SIAH2, SLC25A3, SLC25A6, SLC26A2, SLC38A1, SLC39A6, SLC7A2, SMG5, SPARC, SPTBN1, SREBF2, SRRM2, SSR2, STEAP1, STOM, TAGLN2, TAT, TBC1D16, TFF3, TGOLN2, THAP4, TM9SF2, TMBIM6, TMC5, TMED2, TMEM59, TMEM66, TOB1, TOMM6, TPT1, TRPS1, TSPAN1, TTC39A, TUFM, TXNIP, UCK2, UFC1, UGDH, UNC13B, WIPI1, WNK1, XBP1, ZBTB7B, ZNF207, and any combinations thereof.

In some embodiments, the cancer to be diagnosed or prognosed can be pancreatic cancer. In such embodiments, the methods or assays described herein can be used to distinguish a cancerous pancreas tissue from a normal pancreas tissue, or identify a given state of a cancerous pancreas tissue, e.g., early-stage pancreatic cancer or late-stage pancreatic cancer.

To distinguish a cancerous pancreas tissue from a normal pancreas tissue and/or to determine whether the cancerous pancreas tissue has or is at risk of having early-stage pancreas cancer, in some embodiments, the methods or assays described herein can comprise detecting the presence or absence of a short RNA sequence originating from one or more exons of at least one protein-coding gene, and/or from one or more segments of at least one non-coding transcript. Examples of protein-coding genes whose exons are pertinent for early-stage pancreatic cancer can include, without limitations, ACTG1, ALB, AMY2B, C7, CEL, CELA3A, CLPS, COL3A1, CPA1, CPA2, CPB1, CTRB1, CTRB2, CUZD1, EEF2, GANAB, GATM, GP2, HDLBP, KHDRBS1, KLK1, KRT7, OLFM4, P4HB, PLA2G1B, PPDPF, PRSS1, PRSS3, REG1A, REG1B, REG3A, RNASE1, RPL8, SPINK1, SYCN, UNC13B, and any combinations thereof.

To distinguish a cancerous pancreas tissue from a normal pancreas tissue and/or to determine whether the cancerous pancreas tissue has or is at risk of having late-stage pancreas cancer, in some embodiments, the methods or assays described herein can comprise detecting the presence or absence of a short RNA sequence originating from one or more exons of at least one protein-coding gene, and/or from one or more segments of at least one non-coding transcript. Examples of protein-coding genes whose exons are pertinent for late-stage pancreatic cancer can include, without limitations, ACTB, ANXA2, ANXA5, APOE, ATP6VOC, C1QA, C1QB, C1QC, C1S, CALR, CCNI, CD14, CD44, CD59, CD68, COL1A2, COL6A3, CTSB, CTSC, EEF2, F13A1, FLNA, FN1, GLUL, GPNMB, GPX1, HIST1H2BD, IGFBP4, IGHM, IGKC, ISG15, LAMB3, LAPTM5, LGALS3BP, METTL7A, MMP11, MMP14, MT-CO2, MT-CYB, MYH9, OAZ1, P4HB, PLEC, PSAP, RNASE1, RPN1, SAT1, SERPINA1, SERPING1, SLC40A1, SLCO2B1, SPP1, SRGN, TGM2, TGOLN2, TIMP2, TXNIP, VSIG4, ZYX, and any combinations thereof.

In some embodiments of any aspects described herein, a short RNA sequence can be originated from one or more exons of a protein-coding gene, the protein encoded by which is not present or the expression of which is not detectable in a biological sample. For example, even a given protein may not be present or detectable in a biological sample, short RNAs that originate from one or more exons that would normally make the mRNA of the protein can be present and/or detectable, and thus can be used as biomarker for diagnostic or prognostic methods and/or systems described herein.

For a subject who is determined to have, or is at risk of developing, or is at a given stage of cancer (e.g., breast carcinoma or pancreatic carcinoma), the subject can be administered or prescribed with a specific treatment, e.g., chemotherapy, radiation therapy, surgery, engineered transcripts that can “sponge” various combinations of the short RNAs described herein, or any combinations thereof.

### Short RNA Sequences or Molecules

As used herein, the term “a short RNA sequence” or “a short RNA molecule” generally refers to a nucleic acid sequence or molecule (e.g., RNA, or cDNA) having a distinct sequence of nucleotides, at least part of which corresponds to a segment of an exon of a protein-coding gene, or a segment of a non-coding transcript (e.g., non-protein coding RNA). In some embodiments, a short RNA sequence or short RNA molecule can refer to a nucleic acid sequence or molecule (e.g., RNA or cDNA) having a sequence of nucleotides, at least about 30% of which corresponds to a segment of an exon of a protein-coding gene or a segment of a non-coding transcript. For example, at least 30%, including at least about 40%, at least about 50%, at least about 60%, at least about 70%, at least about 80%, at least about 90%, at least about 95%, at least about 98%, up to and including 100%, of a short RNA sequence or molecule corresponds to a segment of an exon of a protein-coding gene or a segment of a non-coding transcript. In one embodiment, the term “a short RNA sequence” or “a short RNA molecule” refers to a nucleic acid sequence or molecule (e.g., RNA or cDNA) having substantially the entire sequence corresponding to a segment of an exon of a protein-coding gene or a segment of a non-coding transcript.

In some embodiments, the short RNA sequence is not a miRNA. As used herein, the term “miRNA” is a RNA molecule with a length of about 19 about 23 nucleotides whose generation has followed the currently known biogenesis pathways (including but not limited to transcription and processing of a miRNA precursor, splicing of appropriately sized introns, processing of a tRNA transcript, etc). miRNAs act on mRNAs in a sequence-specific manner and can generally block translation of the target mRNA or facilitate its degradation. In one embodiment, the short RNA sequence is not originated from an intron. In some embodiments, the short RNA sequence is not originated or generated from an miRNA precursor.

In some embodiments, the short RNA sequence is not a piRNA. As used herein, the term “piRNA” is short for PIWI-interacting RNA. piRNAs are generally composed of about 26 nucleotides to about 31 nucleotides, and so far have been found in germ cells. piRNAs exhibit a bias for a 5′ uridine and/or a 2′-0-methylation at their 3′ end. In one embodiment, the short RNA sequence is not produced in a germ cell. In some embodiments, the short RNA sequence is produced in a somatic cell.

In some embodiments, the short RNA sequence is not a siRNA. As used herein, the term “siRNA” is short for small interfering RNAs, and refers to a double-stranded RNA molecule of about 20-25 basepairs that can be either exogenous (e.g., synthetic) or endogenous as was reported in plants and in both invertebrate and vertebrate animals.

In some embodiments, a short RNA sequence or short RNA molecule can have at least a portion of sequence corresponding to at least about 5%, at least about 10%, at least about 20%, at least about 30%, at least about 40%, at least about 50%, at least about 60%, at least about 70%, at least about 80%, at least about 90%, at least about 95%, at least about 98%, up to and including 100%, of an exon sequence of a protein-coding gene or a segment of a non-coding transcript.

A short RNA sequence or molecule having a nucleic acid sequence corresponding to a segment of an exon of a protein-coding gene or a segment of a non-coding transcript can, directly or indirectly, originate from at least a segment of an exon of a protein-coding gene or at least a segment of a non-coding transcript. In some embodiments, a short RNA sequence or molecule can be produced by direct transcription of at least a segment of an exon of a protein-coding gene. In some embodiments, a short RNA sequence or molecule can be produced by cleaving from a longer RNA transcript. In some embodiments, a short RNA sequence or molecule can be produced by splicing from a longer RNA transcript. In some embodiments, a short RNA sequence or molecule can be produced by copying from a longer RNA transcript.

During detection in a biological sample, the short RNA sequence can be reverse-transcribed to cDNA for analysis. In such embodiments, a short RNA sequence or molecule can be a nucleic acid sequence (e.g., cDNA) complementary to the short RNA sequence.

As used herein, the term “exon” generally refers to a nucleic acid sequence that at least a portion of which encodes a protein or a portion thereof. For example, an exon can be wholly or part of the protein-coding sequence. Alternatively, an exon can include both sequences that code for amino acids and untranslated regions. In some embodiments, an exon can be a sequence corresponding to wholly or part of the 5′ untranslated region (5′ UTR) or the 3′ untranslated region (3′ UTR) of a protein-coding gene.

Without wishing to be bound by theory, the term “exon” is generally used to refer to a block of contiguous locations of a DNA sequence that can be transcribed as well as to the RNA that can result from the transcription of the corresponding DNA sequence. When present in an RNA molecule, an exon can be adjacent to one or more introns (prior to splicing) or to one or more exons (after splicing). On occasion, two or more adjacent exons can originate from different precursor RNA molecules that have been ligated, e.g., chimeric or fusion transcripts resulting from trans-splicing. In some embodiments, the term “exon” has been used to refer to identifiable components of longer transcripts that may not necessarily give rise to protein-coding products. For example, a long non-coding RNA can comprise a single exon. Alternatively, a long non-coding RNA can comprise one or more introns and two or more exons separated by the one or more introns: following a splicing step, the two or more exons are joined into a single transcript that may or may not code for an amino acid sequence.

The short RNA sequence can have a length less than about 200 nucleotides. In some embodiments, the short RNA sequence can have a length of about 5 nucleotides to about 200 nucleotides. In some embodiments, the short RNA sequences can have a length of about 10 nucleotides to about 100 nucleotides. In some embodiments, the short RNA sequence can have a length of about 15 nucleotides to about 50 nucleotides. In some embodiments, the short RNA sequence can have a length of about 18 nucleotides to about 40 nucleotides. In some embodiments, the short RNA sequence can have a length of about 30 nucleotides to about 40 nucleotides. In some embodiments, the short RNA sequence can have a length of about 32 nucleotides to about 50 nucleotides. In some embodiments, the short RNA sequence can have a length of about 32 nucleotides to about 40 nucleotides. In some embodiments, the short RNA sequence can have a length of about 10 nucleotides to about 40 nucleotides. In some embodiments, the short RNA sequence can have a length of about 15 nucleotides to about 35 nucleotides. In some embodiments, the short RNA sequence can have a length of about 17 nucleotides to about 30 nucleotides. In some embodiments, the short RNA sequence can have a length of about 34 nucleotides.

The short RNA molecule can exist as single-stranded or at least partially double-stranded (e.g., self-hybridizing structures). In one embodiment, the short RNA sequence exists as single-stranded. By way of example only, exemplary short RNA sequences originating from different exons of a protein-coding gene ELOVL5 can include, but are not limited to, AAATCTAGTGGAACAGTCAGTTTAACTTTTTAAC (SEQ ID NO: 1) or TTACTATGGTTTGTCGTCAGTCCCTTCCATGCGT (SEQ ID NO: 2), or can include fragments of these sequences, or can include these sequences as substrings.

Other exemplary short RNA sequences originating from one or more exons of the protein-coding gene ELOVL5 can include, but are not limited to, ATGTGAAATCAGACACGGCACCTTCA (SEQ ID NO: 3), or AAATCTAGTGGAACAGTCAGTTTAACTTTTTAAC (SEQ ID NO: 4), or ATTTGAGGCAGTGGTCAAACAGGTAAAGC (SEQ ID NO: 5), or TATGAGTTGTGCCCCAATGC (SEQ ID NO: 6), or TACAATGTTGTTATGGTAGAGAAACACACATGCC (SEQ ID NO: 7), or CTATTGGCTTTGAATCAAGCAGGCTC (SEQ ID NO: 8), or TGTATGTCTTCATTGCTAGG (SEQ ID NO: 9), or TCCAAACCACGTCATCTGATTGTAAGCA (SEQ ID NO: 10), or GCCTATGATGTGTGTCATTTTAAAGTGTCGGA (SEQ ID NO: 11), or CACGTCATCTGATTGTAAGCAC (SEQ ID NO: 12), or AAGCTGCGGAAGGATTGAAGTCAAAGAATT (SEQ ID NO: 13), or TAAAGCCTATGATGTGTGTCATTT (SEQ ID NO: 14), or GGGTCTAAATTTGGATTGATTTATGCAC (SEQ ID NO: 15), or AGATTTCTAACATTTCTGGGCTCTCTGACC (SEQ ID NO: 16), or AAGCAAAGTGTAAATCAGAGGTTTAAGTTAAAAT (SEQ ID NO: 17), or TGATTCATGTAGGACTTCTTTCATCAATTCAAAA (SEQ ID NO: 18), or GTGTCATTTTAAAGTGTCGGAATTTAGCCTCT (SEQ ID NO: 19), or GTGGGTTTTCTGTTTGAAAAGGAG (SEQ ID NO: 20), or GACACGGCACCTTCAGTTTTGTACTAT (SEQ ID NO: 21), or CATAAGAGAATCGAGAAATTTGATAGAGGT (SEQ ID NO: 22), or CAGCATAAGAGAATCGAGAAA (SEQ ID NO: 23), or AAGCTTATTAGTTTAAATTAGGGTATGTTTC (SEQ ID NO: 24), or TGTCTAAACAGTAATCATTAAAACATTTTTGATT (SEQ ID NO: 25), or TAGACTGCTTATCATAAAATCACATC (SEQ ID NO: 26), or CTTAGCTCACCTGGATATAC (SEQ ID NO: 27), or CGTAGATGAGCAATGGGGAAC (SEQ ID NO: 28), or ATGTAGGACTTCTTTCATCAATTCAAAACC (SEQ ID NO: 29), or ATGCTTTAATTTTGCACATTCGTACTATAGGGAG (SEQ ID NO: 30), or ATAAGATTTCTAACATTTCTGGGCTCTCTGACCC (SEQ ID NO: 31), or AGGTAAAATCAAATATAGCTACAGC (SEQ ID NO: 32), or AGAGATGATTGCCTATTTACC (SEQ ID NO: 33), or AACCCCTAGAAAACGTATAC (SEQ ID NO: 34), or AACATTTCTGGGCTCTCTGACCCCTGCG (SEQ ID NO: 35), or TTATCATAAAATCACATCTCACACATTTGAGGC (SEQ ID NO: 36), or TGGATATACCTACATTGTTAAATGTC (SEQ ID NO: 37), or TGCTTTAATTTTGCACATTCGTACTATAGGGAGCC (SEQ ID NO: 38), or GGGTCTAAATTTGGATTGATTTATGC (SEQ ID NO: 39), or GGCACCTTCAGTTTTGTACTATTGGCTTTGAATC (SEQ ID NO: 40), or GCACCTTCAGTTTTGTACTATTGGCTTTGAATCAA (SEQ ID NO: 41), or CGTCATCTGATTGTAAGCACAATATGAGTTGTGCC (SEQ ID NO: 42), or CCTCCAAACCACGTCATCTGATTGTAAGCACAAT (SEQ ID NO: 43), or ACATTTCTGGGCTCTCTGACCCC (SEQ ID NO: 44), or AACCCCTAGAAAACGTA (SEQ ID NO: 45), or TTTAGAAAAAATCAAAGACCATGATTTATGAAAC (SEQ ID NO: 46), or TCGTGATGAAACTTAAATATATATTCTTTGTC (SEQ ID NO: 47), or GTGTGATTCATGTAGGACTTC (SEQ ID NO: 48), or GGGCTCTACAGCAGTCGTGATGAAACTTAAATAT (SEQ ID NO: 49), or GCCTTAAAATTTAAAAAGCAGGGCCCAAAGCTTA (SEQ ID NO: 50), or GCCTTAAAATTTAAAAAGCAGGGCCCAAAGC (SEQ ID NO: 51), or GCACCTTCAGTTTTGTACTATTGGCTTTGAATCA (SEQ ID NO: 52), or GAAAGGGAGTATTATTATAGTATAC (SEQ ID NO: 53), or CTCACACATTTGAGGCAGTGG (SEQ ID NO: 54), or ATAGTACTTGTAATTTCTTTCTGCTTAGAATC (SEQ ID NO: 55), or AGGTAAAATCAAATATAACTACAGC (SEQ ID NO: 56), or AGATTTCCTTGTAAAATGTG (SEQ ID NO: 57), or ACCACGTCATCTGATTGTAAGC (SEQ ID NO: 58), or ACAGGTAAAGCCTATGATGTGTGT (SEQ ID NO: 59), or AATATGAGTTGTGCCCCAATGCTCG (SEQ ID NO: 60), or AACTAATGTGACATAATTTCCAGTGA (SEQ ID NO: 61), or TGGAAAGGGAGTATTATTATAGTATACAACACTG (SEQ ID NO: 62), or TGACTTGTTGATGTGAAATCAGACAC (SEQ ID NO: 63), or TACAGCATAAGAGAATCGAGAAATTTGATAGAGG (SEQ ID NO: 64), or GTTATAACATGATAGGTGCTGAATT (SEQ ID NO: 65), or GTAAATCTAATAGTACTTGTAATTTCTTTCTGCT (SEQ ID NO: 66), or GGTAAAGCCTATGATGTGTGTCATTTTAAAGTGTCG (SEQ ID NO: 67), or GGTAAAGCCTATGATGTGTGTCATTTTAAAGTGT (SEQ ID NO: 68), or GGGCTCTACAGCAGTCGTGATGAAACTTAAATATATATTCT (SEQ ID NO: 69), or GCGAGAGAGGATGTATACTTTTCAAGAGAGATGA (SEQ ID NO: 70), or CTAGTGGAACAGTCAGTTTAAC (SEQ ID NO: 71), or ATGGTAGAGAAACACACATGC (SEQ ID NO: 72), or ATGCTTTAATTTTGCACATTCGTACTATAGGGAGC (SEQ ID NO: 73), or ATCAATTCAAAACCCCTAGAAAACGTATACAG (SEQ ID NO: 74), or ATAAGATTTCTAACATTTCTGGGCTCTCTGACCCCT (SEQ ID NO: 75), or AGAAACACACATGCCTT (SEQ ID NO: 76), or ACCACGTCATCTGATTGTAAGCACAATATGAGTTC (SEQ ID NO: 77), or AAGCCTATGATGTGTGTCATTTTAAAGTGTCGGA (SEQ ID NO: 78), or AAATCTAGTGGAACAGTCAGTTTAACTTTTTAACAGA (SEQ ID NO: 79), or AAACCACGTCATCTGATTGTAAGC (SEQ ID NO: 80), or can include fragments of these sequences, or can include these sequences as substrings.

In some embodiments, a short RNA sequence can have an overlapping region with a pyknon. Pyknons are sequences found to repeat in genomic DNA. Pyknons have at least 30 or more instances in the intergenic and/or intronic sequences of a genome and at least one additional instance in the untranslated and/or protein-coding regions of a gene. Additional details about pyknons and techniques for identifying pyknons can be found, e.g., in Rigoutsos I. et al., “Short blocks from the noncoding parts of the human genome have instances within nearly all known genes and relate to biological processes.” PNAS (2006) 103: 6605-6610; U.S. App. No. US 2007/0042397, and U.S. Pat. No. 8,065,091, the contents of which are incorporated herein by reference.

In one embodiment, a short RNA sequence can be originated from at least a portion of an exon of a messenger RNA.

In one embodiment, a short RNA sequence can be originated from at least a portion of a region or a genomic locus that would normally give rise to a long (non-coding) transcript (e.g., non-protein-coding RNA).

### Exemplary Methods for Detecting a Short RNA Sequence

Short RNA sequence(s) can be detected by any methods known in the art, including, but not limited to, Sanger sequencing, polymerase chain reaction (PCR), and real-time quantitative PCR, northern blot, microarray, in situ hybridization, serial analysis of gene expression (SAGE), cap analysis gene expression (CAGE) and massively parallel signature sequencing (MPSS), next generation sequencing (including deep sequencing, e.g., sequencing with deep coverage), direct multiplexing, etc., and any combinations thereof.

Methods for performing SAGE to detect RNAs have been previously described in Velculescu, V. E. et al. “Serial Analysis of Gene Expression.” Science (1995) 270: 484-487; and Saha S. et al. “Characterization of the yeast transcriptome” Cell (1997) 88: 243-251, and exemplary SAGE protocols can be accessed at sagenet.org/protocol/index.htm. Methods for performing CAGE to detect RNAs has been previously described, e.g., in Kodzius R. et al. “CAGE: cap analysis of gene expression” Nature Methods (2006) 3: 211-222. Methods for performing MPSS to detect RNAs can be found, e.g., in Brenner, S. et al. “Gene expression analysis by massively parallel signature sequencing (MPSS) on microbead arrays” Nature Biotechnology (2000) 18: 630-634.

In some embodiments, the INVADER® assay (Third Wave Technologies Inc., Madison, Wis.) can be modified and used to detect short RNA sequences in a biological sample. The INVADER® assay is generally a homogeneous, isothermal, signal amplification system for the quantitative detection of nucleic acids. The assay can directly detect either DNA or RNA without target amplification or reverse transcription. It is based on the ability of Cleavase® enzymes to recognize as a substrate and cleave a specific nucleic acid structure generated through the hybridization of two oligonucleotides to the target sequence. Modification of the INVADER® assay for short RNA sequence detection has been previously described, e.g., in de Arruda, M. et al. “Invader technology for DNA and RNA analysis: Principles and applications” Expert Rev. Mol. Diagn (2002) 2: 487-496; Eis, P. S. et al. “An invasive cleavage assay for direct quantitation of specific RNAs” Nat. Biotechnol. (2001) 19: 673-676; and Allawi H. T. et al. “Quantitation of microRNAs using a modified Invader assay” RNA (2004) 10: 1153-1161.

Next-generation sequencing (NGS) is a novel approach for the detection and sequencing of DNA or RNA molecules as reviewed, e.g., in Voelkerding K. V. et al. “Next-Generation Sequencing From Basic Research to Diagnostics” Clinical Chemistry (2009) 55: 641-658; Metzker M. L. “Sequencing technologies—the next generation” Nature Reviews (2010) 11: 31-46; Zhang J. et al. “The impact of next-generation sequencing on genomics” J. Genet Genomics (2011) 38: 95-109; and Pareek C. S. et al. “Sequencing technologies and genome sequencing” J. Appl Genetics (2011) 52: 413-435. Various commercial NGS instruments and reagent kits for high-throughput next-generation sequencing have been developed and used for RNA-sequencing. For example, exemplary NGS instruments that can be used for RNA-sequencing or deep sequencing of RNA can include, but are not limited to, the GS FLX sequencer (based on pyrosequencing) from 454 Life Sciences now part of ROCHE Diagnostics [http://www.454.com/], the Genome Analyzer (based on polymerase-based sequence-by-synthesis) from Illumina [http://www.illumina.com], the SOLiD™ System (based on ligation-based sequencing) from Applied Biosystems [http://www.appliedbiosystems.com/absite/us/en/home/applications-technologies/solid-next-generation-sequencing/next-generation-systems.html], and the HeliScope™ Single Molecule Sequencer from Helicos BioScience [http://www.helicosbio.com/].

Other NGS or higher-generation sequencing methods based on single-molecule sequencing (without PCR amplification) can also be used to detect short RNA sequences or molecules in some embodiments of the methods, assays, and systems described herein. Examples of single-molecule sequencing methods can include, but are not limited to, Ion Torrent (pH sensing), nanopore sequencing, and transmission electron microscope (TEM) for sequencing. See, e.g., Perkel, J. “Making contact with Sequencing's Fourth Generation” BioTechniques (2011): 50:93-95.

An Exemplary Method for Identifying Short RNA Sequences and/or a Protein-Coding Gene that Gives Rise to Short RNAs Out of at Least One Exon or of at Least One Segment of a Non-Coding Transcript (e.g., a Non-Coding RNA)

In contrast to most of the RNA detection methods described herein that generally need prior sequence information for design of probes or primers to detect target nucleic acid sequences, such as microarray and realtime-PCR, next- or higher-generation sequencing (including deep sequencing) does not require any prior information of the sequences, and thus allows for discovery of novel short RNA sequences present in a biological sample. New short RNA sequence information provided by deep sequencing can then be used to design microarray probe content or primers for expression studies.

Accordingly, in some embodiments of the methods, assays and systems described herein, next- or higher-generation sequencing (including deep sequencing or RNA sequencing) can be used to detect and discover in a biological sample short RNA sequences originating from one or more exons of at least one specific protein-coding gene, and/or from one or more segments of at least one non-coding transcript in a state-specific and tissue-specific manner. For example, after sequencing, the sequenced reads can be mapped on the assembly of a reference genome (e.g., a human genome which can be assessed at hgdownload.cse.ucsc.edu/downloads.html#human.) using any of the bioinformatics tools described herein, e.g., short-read mapping tools such as BWA, SHRiMP, Bowtie, and the like. If a sequenced read is mapped at multiple locations of the genome, then all instances of the read are preferably discarded. This can ensure that any of the genomic location that gives rise to the sequenced RNA read can be unambiguously determined.

After mapping to a reference genome, each sequenced read set can generate a genomic map that shows the originating location of the short RNA molecules. The genomic maps can be visualized with any genomic browser known in the art, e.g., the Univ. of California at Santa Cruz Genome Browser (which can be assessed at genome.ucsc.edu/cgi-bin/hgGateway). Other genomic browsers that can be used include, but are not limited to EagleView, LookSeq, MapView, Sequence Assembly Manager, STADEN, and XMatchView.

A specific computer program can then be used to analyze a genomic map obtained from profiling of short RNAs in the biological sample. For example, in the program, the genomic map can be intersected with coordinates of the exons of any known protein-coding genes, which will in turn generate a collection of “islands” that (a) overlap protein-coding exons and (b) generate short RNAs. Then, by sliding a window across each of these islands, it can be determined whether a significant fraction of the window's span gives rise to short RNAs and whether for a given window offset the change in amount of the short RNAs between at least two reference samples of different states (e.g., normal sample vs. diseased sample) exceeds a certain threshold (e.g., a significant difference between a normal sample and a diseased sample). This can allow identifying, for example, a protein-coding gene, one or more exons of which satisfy these requirements. At the same time, this can also allow identifying one or more short RNA sequences that can distinguish a normal sample from a diseased or abnormal sample, or between diseased or abnormal samples of different stages.

Some exemplary protein-coding genes that can give rise to short RNA molecules to distinguish a cancer sample from a normal sample are identified and shown in Examples 2-7. Additionally, specific short RNA sequences from a protein-coding gene can be identified and used as biomarkers for determining a state of a corresponding cell or tissue and/or a state of disease or disorder. For example, a short RNA sequence that shows a significantly different amount in a normal sample than in a cancer sample can be used as a biomarker for distinguishing a cancer cell from a normal cell. As shown in Example 8, two exemplary sequences of the short RNA molecules, respectively, located in the 3′ UTR (B1) and the CDS (B2) regions of ELOVL5 are indicated below:

| (SEQ ID NO. 1) | |
|---|---|
| B1 (3′UTR): | AAATCTAGTGGAACAGTCAGTTTAACTTTTTAAC |
| (SEQ ID NO. 2) | |
| B2 (CDS): | TTACTATGGTTTGTCGTCAGTCCCTTCCATGCGT |

Additionally, a short RNA sequence that shows a significantly different amount between two distinct stages of a cancer (e.g., DCIS vs. INV) can be used as a biomarker for distinguishing one state of a cancer cell from another state.

After identification of the short RNA sequences for determining a given state of a cell or a tissue, microarray probes or primers can be designed accordingly to detect such short RNA sequences in a biological sample, using any probe-based or hybridization-based detection methods described herein, e.g., but not limited to, Northern blots, real-time PCR, and microarrays. Methods for designing probes or primers for hybridization-based detection methods are known in the art.

One method to detect short RNA sequences can employ microarrays with probes designed to capture short RNA sequences. Accordingly, a microarray having at least one oligonucleotide probe appended thereon, can be used for detection of short RNA molecules. Probes can be affixed to solid support surfaces for use as “microarrays.” Such microarrays can be used to detect expression of short RNA sequences in a biological sample by a number of techniques known to one of skill in the art. In one technique, oligonucleotides targeting short RNA molecules or cDNAs of the short RNA molecules are arrayed on a microarray for determining the RNA sequence by hybridization approach, such as that outlined in Malone J. H. and Oliver B. “Microarrays, deep sequencing and the true measure of the transcriptome” BMC Biology (2011) 9: 1-9. The oligonucleotide probes can also be used for fluorescent detection of a short RNA sequence. See, e.g., Nelson PT. et al. “Microarray-based, high-throughput gene expression profiling of microRNAs.” Nat Methods (2004) 1: 155-161. A probe also can be affixed to an electrode surface for the electrochemical detection of short RNA sequences such as described by Pöhlmann C. and Sprinzl M. “Electrochemical Detection of MicroRNAs via Gap Hybridization Assay” Anal. Chem. (2010) 82: 4434-4440. For example, a gap hybridization assay based on four components DNA/RNA hybridization and electrochemical detection using esterase 2-oligodeoxynucleotide conjugates can be used to detect short RNA sequences or molecules. Complementary binding of short RNA sequences to a gap built of capture and detector oligodeoxynucleotide, the reporter enzyme is brought to the vicinity of the electrode and produces enzymatically an electrochemical signal. In the absence of a short RNA sequence, the gap between capture and detector oligodeoxynucleotide is not filled, and missing base stacking energy destabilizes the hybridization complex.

In general, the PCR procedure describes a method of gene amplification which is comprised of (i) sequence-specific hybridization of primers to specific genes within a nucleic acid sample or library, (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

In an alternative embodiment, an amount of one or more short RNA sequences or molecules described herein can be determined by reverse-transcription (RT) PCR and by quantitative RT-PCR (QRT-PCR) or real-time PCR methods. Methods of RT-PCR and QRT-PCR are well known in the art.

In one embodiment, an amount of one or more short RNA sequences or molecules described herein can be determined by in situ hybridization (e.g., on a biopsy), the method of which, e.g., is described in Pena J. T. G. et al. “miRNA in situ hybridization in formaldehyde and EDC—fixed tissues.” Nature Methods (2009) 6: 139-141.

### Systems and Computer Readable Media for Determination of a Given State of a Cell or Tissue

Another aspect provided herein relates to systems (and computer readable media for causing computer systems) to perform a method for determining a given state of a cell or a tissue and/or diagnosing or prognosing a disease or disorder, or a state of a disease or disorder, based on presence and/or absence of a short RNA sequence (including an expression level of short RNA sequence) originating from at least part of an exon of a protein-coding gene.

A system for analyzing a biological sample is provided. The system comprises: (a) a determination module configured to receive a biological sample and to determine sequence information, wherein the sequence information comprises a sequence of a short RNA molecule originating from an exon of at least one protein-coding gene, and/or from a segment of at least one non-coding transcript; (b) a storage device configured to store sequence information from the determination module; (c) a comparison module adapted to compare the sequence information stored on the storage device with reference data, and to provide a comparison result, wherein the comparison result identifies the presence or absence of the short RNA molecule, wherein a discrepancy in an expression level or in an originating location of the short RNA molecule from the reference data is indicative of the biological sample having an increased likelihood of having or being at a cellular or tissue state different from a state represented by the reference data; and (d) a display module for displaying a content based in part on the comparison result for the user, wherein the content is a signal indicative of a subject having, or being at risk of developing, or being at a given stage of a disease or disorder, or a signal indicative of lacking a disease or disorder.

A computer readable medium having computer readable instructions recorded thereon to define software modules including a comparison module and a display module for implementing a method on a computer is further provided herein. The computer-readable physical medium having computer readable instructions recorded thereon to define software modules includes a comparison module and a display module for implementing a method on a computer, wherein the method comprises: (a) comparing with the comparison module the data stored on a storage device with reference data to provide a comparison result, wherein the comparison result the comparison result identifies the presence or absence of the short RNA molecule, wherein a discrepancy in an expression level or in an originating location of the short RNA molecule from the reference data is indicative of the biological sample having an increased likelihood of having or being at a cellular or tissue state different from a state represented by the reference data; and (b) a display module for displaying a content based in part on the comparison result for the user, wherein the content is a signal indicative of a subject having, or being at risk of developing, or being at a given stage of a disease or disorder, or a signal indicative of lacking a disease or disorder.

Embodiments provided herein have been described through functional modules, which are defined by computer executable instructions recorded on computer readable media and which cause a computer to perform method steps when executed. The modules have been segregated by function for the sake of clarity. However, it should be understood that the modules need not correspond to discrete blocks of code and the described functions can be carried out by the execution of various code portions stored on various media and executed at various times. Furthermore, it should be appreciated that the modules may perform other functions, thus the modules are not limited to having any particular functions or set of functions.

The computer readable media can be any available tangible media that can be accessed by a computer. Computer readable media includes volatile and nonvolatile, removable and non-removable tangible media implemented in any method or technology for storage of information such as computer readable instructions, data structures, program modules or other data. In some embodiments, computer readable media excludes transitory form of signal transmission, e.g., electromagnetic waves. Computer readable media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable media, or computer readable medium 200, can define instructions, for example, as part of one or more programs, that, as a result of being executed by a computer, instruct the computer to perform one or more of the functions described herein (e.g., in relation to system 10, or computer readable medium 200), and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of system 10, or computer readable medium 200 described herein, can be distributed across one or more of such components, and may be in transition there between.

The computer-readable media can be transportable such that the instructions stored thereon can be loaded onto any computer resource to implement the aspects provided herein. In addition, it should be appreciated that the instructions stored on the computer readable media, or computer-readable medium 200, described above, are not limited to instructions embodied as part of an application program running on a host computer. Rather, the instructions can be embodied as any type of computer code (e.g., software or microcode) that can be employed to program a computer to implement various aspects described herein. The computer executable instructions can be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2nd ed., 2001).

The functional modules of certain embodiments provided herein include a determination module, a storage device, a comparison module and a display module. The functional modules can be executed on one, or multiple, computers, or by using one, or multiple, computer networks. The determination module 40 has computer executable instructions to provide sequence information in computer readable form. As used herein, “sequence information” refers to any nucleotide sequence, including but not limited to full-length sequence, partial sequence, or mutated sequences. Moreover, information “related to” the sequence information includes detection of the presence or absence of a short RNA sequence, determination of the expression level of a short RNA sequence in a biological sample, and the like. In some embodiments, the sequence information can include sequences of any short RNA molecules present in a biological sample. In some embodiments, the sequence information can include sequences of short RNA molecules originating from one or more exons of at least one protein-coding gene, and/or from one or more segments of at least one non-coding transcript. In other embodiments, the sequence information can include sequences of short RNA molecules described herein, miRNA molecules, piRNA molecules, mRNA molecules, or any combinations thereof. In some embodiments, the sequence information can include sequences of short RNA molecules present in a biological sample, and a genomic sequence of one or more protein-coding genes.

As an example, determination modules 40 for determining sequence information can include known systems for automated sequence analysis, including but not limited to, Hitachi FMBIO® and Hitachi FMBIO® II Fluorescent Scanners (available from Hitachi Genetic Systems, Alameda, Calif.); Spectrumedix® SCE 9610 Fully Automated 96-Capillary Electrophoresis Genetic Analysis Systems (available from SpectruMedix LLC, State College, Pa.); ABI PRISM® 377 DNA Sequencer, ABED 373 DNA Sequencer, ABI PRISM® 310 Genetic Analyzer, ABI PRISM® 3100 Genetic Analyzer, and ABI PRISM® 3700 DNA Analyzer (available from Applied Biosystems, Foster City, Calif.); Molecular Dynamics FluorImager™ 575, SI Fluorescent Scanners, and Molecular Dynamics FluorImager™ 595 Fluorescent Scanners (available from Amersham Biosciences UK Limited, Little Chalfont, Buckinghamshire, England); GenomyxSC™ DNA Sequencing System (available from Genomyx Corporation (Foster City, Calif.); and Pharmacia ALF™ DNA Sequencer and Pharmacia ALFexpress™ (available from Amersham Biosciences UK Limited, Little Chalfont, Buckinghamshire, England); any next- or higher-generation sequencing instruments such as, but not limited to, GF GLX Titanium, GS Junior (available from 454 Life Sciences, part of Roche Diagnostic Corporation, Branford, Conn.); HiSeq 2000, Genome Analyzer IIX, Genome Analyzer IIE, iScan SQ (available from Illumina, San Diego, Calif.); ABI SOLiD™ system (e.g., SOLiD4 platform available from Life Technologies, Applied Biosystems, Carlsbad, Calif.); HeliScope™ Single Molecule Sequencer (available from Helicos Biosciences Corporation, Cambridge, Mass.); and PACBIO RS (available from Pacific Biosciences, Menlo Park, Calif.).

Alternative methods for determining sequence information, i.e., determination modules 40, include systems for nucleic acid analysis. For example, mass spectrometry systems including Matrix Assisted Laser Desorption Ionization—Time of Flight (MALDI-TOF) systems and SELDI-TOF-MS ProteinChip array profiling systems; systems for analyzing gene expression data (see, for example, published U.S. Patent Application, Pub. No. U.S. 2003/0194711); systems for array based expression analysis: e.g., HT array systems and cartridge array systems such as GeneChip® AutoLoader, Complete GeneChip® Instrument System, GeneChip® Fluidics Station 450, GeneChip® Hybridization Oven 645, GeneChip® QC Toolbox Software Kit, GeneChip® Scanner 3000 7G plus Targeted Genotyping System, GeneChip® Scanner 3000 7G Whole-Genome Association System, GeneTitan™ Instrument, and GeneChip® Array Station (each available from Affymetrix, Santa Clara, Calif.); Densitometers (e.g. X-Rite-508-Spectro Densitometer® (available from RP Imaging™, Tucson, Ariz.), The HYRYS™ 2 HIT densitometer (available from Sebia Electrophoresis, Norcross, Ga.); automated Fluorescence in situ hybridization systems (see for example, U.S. Pat. No. 6,136,540); 2D gel imaging systems coupled with 2-D imaging software; microplate readers; Fluorescence activated cell sorters (FACS) (e.g. Flow Cytometer FACSVantage SE, (available from Becton Dickinson, Franklin Lakes, N.J.); and radio isotope analyzers (e.g. scintillation counters).

The sequence information determined in the determination module can be read by the storage device 30. As used herein the “storage device” 30 is intended to include any suitable computing or processing apparatus or other device configured or adapted for storing data or information. Examples of electronic apparatus suitable for use with various embodiments described herein can include stand-alone computing apparatus, data telecommunications networks, including local area networks (LAN), wide area networks (WAN), Internet, Intranet, and Extranet, and local and distributed computer processing systems. Storage devices 30 also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device 30 is adapted or configured for having recorded thereon sequence information or expression level information. Such information may be provided in digital form that can be transmitted and read electronically, e.g., via the Internet, on diskette, via USB (universal serial bus) or via any other suitable mode of communication.

As used herein, “expression level information” refers to any nucleotide expression level information, including but not limited to full-length nucleotide sequences, partial nucleotide sequences, or mutated sequences. Moreover, information “related to” the expression level information includes detection of the presence or absence of a sequence (e.g., presence or absence of a nucleotide sequence), determination of the concentration of a sequence in the sample (e.g., nucleotide (RNA or DNA) expression levels), and the like.

As used herein, “stored” refers to a process for encoding information on the storage device 30. Those skilled in the art can readily adopt any of the presently known methods for recording information on known media to generate manufactures comprising the sequence information or expression level information.

A variety of software programs and formats can be used to store the sequence information or expression level information on the storage device. Any number of data processor structuring formats (e.g., text file or database) can be employed to obtain or create a medium having recorded thereon the sequence information or expression level information.

By providing sequence information or expression level information in computer-readable form, one can use the sequence information or expression level information in readable form in the comparison module 80 to compare a specific sequence or expression profile with the reference data within the storage device 30. In some embodiments, the comparison module 80 can also include bioinformatics analysis tools for next-generation sequencing data (e.g., short-read sequence data). Examples of bioinformatics analysis tools for next-generation sequencing (NGS) data can include any commercial NGS analysis packages that are compatible with the sequenced reads obtained from the NGS instrument. The NGS analysis package can include a sequence mapping tool for mapping sequences (e.g., short-read sequences) to a reference genome, sequence assembly tool for de novo assembly of overlapping reads to form contiguous nucleic acid sequence, a genome browser, and any combinations thereof. Examples of short-read alignment tools for mapping short RNA sequences to a reference genome can include, without limitations, Bfast, BioScope, Bowtie, Burrows-Wheeler Aligner (BWA), CLC bio, CloudBurst, Eland/Eland2, Exonerate, GenomeMapper, GnuMap, Karma, MAQ, MOM, Mosaik, MrFAST/MrsFAST, NovoAlign, PASS, PerM, RazerS, RMAP, SSAHA2, Segemehl, SeqMap, SHRiMP, Slider/SliderII, SOAP/SOAP2, Srprism, Stampy, vmatch, ZOOM and any art-recognized alignment tools that can be used to align short-read sequences to a reference genome. In one embodiment, Burrows-Wheeler Aligner (BWA) can be used to map short RNA sequences to a reference genome (e.g., a human genome). Examples of sequence assembly tools include, but are not limited to, ABySS, ALLPATHS, Edena, Euler-SR, SHARCGS, SHARP, SSAKE, Velvet and any other art-recognized assembly tools. Different genome browsers can be used to visualize genomic maps, e.g., generated after sequence alignment to a reference genome.

In one embodiment, the comparison module 80 uses sequence information alignment programs such as BLAST (Basic Local Alignment Search Tool) or FAST (using the Smith-Waterman algorithm) may be employed individually or in combination. These algorithms determine the alignment between similar regions of sequences and a percent identity between sequences.

In some embodiments, the comparison module 80 can also include a search program that can identify a short RNA sequence originating from an exon of a protein-coding gene, and/or a segment of a non-coding transcript. For example, such search program can intersect each genomic map (e.g., generated after mapping short-read sequences to a reference genome using a mapping tool) with coordinates of exons of known protein-coding genes, which can in turn generate a collection of “islands” that (a) overlap protein-coding exons and (b) generate short RNA sequences; then by sliding a window across each of these islands, and for a given placement of the window whether a significant fraction (e.g., at least about 30% or more) of the window's span gives rise to short RNAs can be determined, and/or whether a change in expression levels of these short RNAs between two reference samples of the same tissue (e.g., normal breast vs. diseased or abnormal breast) exceed a certain threshold (e.g., defined by a significant difference between a normal sample and a diseased or abnormal sample). Thus, this search program can allow identification of a number of protein-coding genes that can satisfy these requirements and also identification of short RNA sequence that can distinguish a normal sample from a diseased or abnormal sample and/or different stages of a disease or disorder.

In some embodiments, the comparison module can include a pattern recognition pattern that can be pre-trained with different reference data sets such as data sets comprising profiles of short RNA sequences obtained from different state of a tissue (e.g., normal data set vs. diseased or abnormal data set; or data sets corresponding to different stages of a disease or disorder and a normal data set).

Accordingly, in some embodiments, the comparison module can compare a profile of short RNA sequences of a biological sample determined by the determination module 40 to reference data stored on the storage device 30, and classify the biological sample into a specific state (e.g., normal, diseased or abnormal, and/or a given stage of a disease or disorder). For example, comparison programs can be used to compare an expression level of a short RNA sequence in a biological sample to a reference data expression level (e.g., sequence data from a control/reference sample described herein) and/or profiles of short RNA sequences in a biological sample to reference data expression profiles (e.g., sequence data from a control/reference sample described herein). The comparison made in computer-readable form provides a computer readable comparison result, which can be processed by a variety of means. Content 140 based on the comparison result can be retrieved from the comparison module 80 to indicate a given state of a cell or a tissue, and/or whether a subject has, or is at risk of developing of a disease or disorder, or a given state of the disease or disorder.

In one embodiment, the reference data stored in the storage device 30 to be read by the comparison module 80 is sequence information data obtained from a reference sample described herein or a control biological sample of the same type as the biological sample to be tested. Alternatively, the reference data are a database, e.g., a collection of sequence information data obtained from a plurality of reference samples described herein and control biological samples of the same type as the biological sample to be tested. For example, reference data can include profiles of short RNA sequences that are indicative of a given state of a cell or tissue and/or a disease or disorder of interest or a given state of the disease or disorder. In one embodiment, the reference data can include sequence information of short RNA sequences and/or profiles of short RNA sequences that are indicative of a disease or disorder of interest, e.g., a disease or disorder afflicting a tissue, and or different stages of a disease or disorder of interest, e.g., different stages of cancer. By way of example only, reference data stored in a system for diagnosing and/or prognosing breast cancer can include, but not limited to, (a) profile(s) of short RNA sequences from at least one exon of a protein-coding gene, e.g., but not limited to, ELOVL5, and/or from at least one segment of a non-coding transcript, obtained from one or a group of normal subjects; (b) profile(s) of short RNA sequences from at least one exon of a protein-coding gene, e.g., but not limited to, ELOVL5, and/or from at least one segment of a non-coding transcript, obtained from one or a group of subjects having a given stage of breast cancer (e.g., DCIS, lobular carcinoma in situ, INV, etc.); (c) profile(s) of short RNA sequences from at least one exon of a protein-coding gene, e.g., but not limited to, ELOVL5, and/or from at least one segment of a non-coding transcript, obtained from a normal tissue of the test subject; (d) profile(s) of short RNA sequences from at least one exon of a protein-coding gene, e.g., but not limited to, ELOVL5, and/or from at least one segment of a non-coding transcript, obtained from a diseased or abnormal tissue of the test subject that was previously diagnosed; and (e) any combinations thereof.

In one embodiment, the reference data are electronically or digitally recorded and annotated from databases including, but not limited to GenBank (NCBI) protein and DNA databases such as genome, ESTs, SNPS, Traces, Celara, Ventor Reads, Watson reads, HGTS, and the like; Swiss Institute of Bioinformatics databases, such as ENZYME, PROSITE, SWISS-2DPAGE, Swiss-Prot and TrEMBL databases; the Melanie software package or the ExPASy WWW server, and the like; the SWISS-MODEL, Swiss-Shop and other network-based computational tools; the Comprehensive Microbial Resource database (available from The Institute of Genomic Research). The resulting information can be stored in a relational database that may be employed to determine homologies between the reference data or genes or proteins within and among genomes.

The “comparison module” 80 can use a variety of available software programs and formats for the comparison operative to compare sequence information determined in the determination module 40 to reference data. In one embodiment, the comparison module 80 is configured to use pattern recognition techniques to compare sequence information from one or more entries to one or more reference data patterns. The comparison module 80 can be configured using existing commercially-available or freely-available software for comparing patterns, and may be optimized for particular data comparisons that are conducted. The comparison module 80 provides computer readable information related to the sequence information that can include, for example, detection of the presence or absence of a short RNA sequence; determination of the concentration of a short RNA sequence in the sample, or determination of an expression profile.

The comparison module 80, or any other module described herein, may include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file, which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in a particular preferred embodiment provided herein, users can directly access data (via Hypertext links for example) residing on Internet databases using a HTML interface provided by Web browsers and Web servers. In one embodiment, users can access data residing on Cloud storage.

Various algorithms or software packages are available which are useful for comparing and analyzing sequence information and/or expression data determined in the determination module 40. For example, various software packages for next-generation sequencing (NGS) analysis are available in the commercial and/or public domains. Exemplary software packages for NGS analysis can include, without limitations, sequence alignment tools as discussed above; de novo alignment and/or assembly tools as discussed above; integrated solutions, such as CLCbio Genomics Workbench, Galaxy, Genomatix, JMP Genomics, NExtGENE, SeqMan Genome Analyzer, SHORE, SlimSearch; genome browser (including alignment viewer and/or assembly database) such as EagleView, LookSeq, MapView, Sequence Assembly Manager, STADEN, XMatchView; software packages for transciptomics such as ERANGE, S-Mo.R-Se, MapNext, QPalma, RSAT, TopHat; or any combinations thereof.

In some embodiments, when the sequence information is determined by microarray-based methods, various software packages for microarray analysis can be used, e.g., but not limited to, GeneChip® Sequence Analysis Software (GSEQ), GeneChip® Targeted Genotyping Analysis Software (GTGS) and Expression Console™ Software. Accordingly, depending on methods used to produce sequence information in the determination module 40, various sequence analysis software can be used.

In one embodiment described herein, pattern comparison software is used to compare an expression profile of short RNA sequences to a reference data for determining a given state of a cell or tissue, or whether the expression profiled obtained from a test subject is indicative of a disease or disorder, or a given state of a disease or disorder.

The comparison module 80 provides computer readable comparison result that can be processed in computer readable form by predefined criteria, or criteria defined by a user, to provide a content based in part on the comparison result that may be stored and output as requested by a user using a display module 110. The display module 110 enables display of a content 140 based in part on the comparison result for the user, wherein the content 140 is a signal indicative of a subject having, or being at risk of developing or being at a given stage of a disease or disorder, or a signal indicative of the subject having no risk of the disease or disorder. Such signal, can be for example, a display of content 140 indicative of the presence or absence of increased risk for a disease or disorder, or a given state of a disease or disorder on a computer monitor, a printed page of content 140 indicating the presence or absence of increased risk for a given state of a disease or disorder from a printer, or a light or sound indicative of the presence or absence of increased risk for a given state of a disease or disorder.

The content 140 based on the comparison result can include an expression profile of one or more short RNA sequences determined from the test subject. In one embodiment, the content 140 based on the comparison result can include a comparison of the short RNA expression profile between the test subject and one or more reference samples described herein. In one embodiment, the content 140 based on the comparison result is merely a signal indicative of the presence or absence of an increased risk of a given state of a disease or disorder.

In one embodiment provided herein, the content 140 based on the comparison result is displayed a on a computer monitor. In one embodiment, the content 140 based on the comparison result is displayed through printable media. The display module 110 can be any suitable device configured to receive from a computer and display computer readable information to a user. Non-limiting examples include, for example, general-purpose computers such as those based on INTEL® processor, QUALCOMM® processors, Sun Microsystems processors, Hewlett-Packard processors, any of a variety of processors available from Advanced Micro Devices (AMD) of Sunnyvale, Calif., or any other type of processors (including mobile processors), visual display devices such as tablet computers, flat panel displays, cathode ray tubes and the like, as well as computer printers of various types.

In one embodiment, a World Wide Web browser is used for providing a user interface for display of the content 140 based on the comparison result. It should be understood that other modules described herein can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces. The requests formulated with the user's Web browser are transmitted to a Web application which formats them to produce a query that can be employed to extract the pertinent information related to the sequence information, e.g., but not limited to, display of nucleotide (RNA or DNA) expression levels; or display of information based thereon. In one embodiment, the sequence information of the reference sample data is also displayed.

In one embodiment, the display module 110 displays the comparison result based on sequence information and whether the comparison result is indicative of a disease or disorder, or a given stage of a disease or disorder. For example, in the case of diagnosis of breast cancer, the display module 110 can display the comparison result based on determined sequence information and whether the comparison results is indicative of breast cancer, or a particular stage of breast cancer (e.g., DCIS, lobular carcinoma in situ, INV, etc.).

In one embodiment, the content 140 based on the comparison result that is displayed is a signal (e.g. positive or negative signal) indicative of the presence or absence of an increased risk for a disease or disorder, or a given stage of the disease or disorder, thus only a positive or negative indication may be displayed.

Provided herein therefore relates to systems 10 (and computer readable medium 200 for causing computer systems) to perform methods for determining a given stage of a cell or a tissue, and/or whether a subject has, or is at risk of developing, or is at a given stage of a disease, e.g., cancer, or disorder, based on expression profiles of short RNA sequences originating from one or more exons of at least one protein-coding gene, and/or from one or more segments of at least one non-coding transcript.

System 10, and computer readable medium 200, are merely an illustrative embodiment provided herein for performing methods of determining whether an individual has a specific disease or disorder or a pre-disposition, for a specific disease or disorder based on expression profiles or sequence information, and are not intended to limit the scope described herein. Variations of system 10, and computer readable medium 200, are possible and are intended to fall within the scope described herein.

The modules of the machine, or used in the computer readable medium, may assume numerous configurations. For example, function may be provided on a single machine or distributed over multiple machines.

### A Reference Sample or Reference Data

As used herein, a reference sample can include a normal or negative control, alternatively a disease (or disorder) or positive control, against which biological samples can be compared. Therefore, it can be determined whether the biological sample to be evaluated for a specific disease or disorder, or a stage of a disease or disorder, has measurable difference or substantially no difference, as compared to a reference sample. A normal or healthy sample or tissue refers to a sample or tissue that does not have a disease or disorder to be evaluated.

The reference sample can be obtained from the patient to be diagnosed or prognosed, or from a different subject, who is preferably of same age and/or race.

In one embodiment, the reference sample can be obtained from the same patient at the same time that the biological sample is taken. In one embodiment, the reference sample can be taken from a normal and/or healthy tissue of the same patient. In one embodiment, the reference sample can be taken from a normal and/or healthy tissue, for example tissue taken adjacent to the cancer, such as within 1 or 2 cm diameter from the leading front of the tumor. Alternatively, the reference sample can be taken from an equivalent position in the subject's body. For example, in the case of breast cancer, a reference sample can be taken from any area of the breast which is not cancerous. In another embodiment, the reference sample can be a disease or abnormal sample taken previously from the same patient, against which a new biological sample can be compared to provide an evaluation of the therapeutic treatment efficacy.

In one embodiment, the reference sample can be a sample taken previously, e.g., a sample of the same or a different cancer/tumor, the comparison of which can, for example, provide characterization of the source of the new tumor, and/or progression or development of an existing cancer, such as before, during or after therapeutic treatment. For example, the reference sample can be obtained from a different patient, e.g., it can be a control sample, or a collection of control samples, representing different stages or different types of diseases or disorders. In one embodiment, the reference sample can be a control sample or a collection of control samples, representing different stages of a specific cancer (e.g., cancer staging samples) or different types of cancer, for example those listed herein (i.e., cancer reference samples). Comparison of the biological sample data with data obtained from such cancer staging or cancer reference samples can, for example, allow for the characterization of the assessed cancer to a specific stage and/or type of cancer.

As used herein, the term “reference data” refers to data obtained from a reference sample as described herein, or a collection of reference samples as described herein.

### Biological Sample of a Subject and Preparation Thereof

A “biological sample” subjected to analysis using the methods, assays and systems described herein generally refers to a sample taken or isolated from a subject or a biological organism. In some embodiments, the biological sample contains one or more cells, e.g., tissue culture mammalian cells, cell lysate, a tissue sample from a subject, a homogenate of a tissue sample from a subject or a fluid sample from a subject. Exemplary biological samples include, but are not limited to, blood (including whole blood, serum, cord blood, and plasma), sputum, urine, spinal fluid, pleural fluid, nipple aspirates, lymph fluid, the external sections of the skin, respiratory, intestinal, and genitourinary tracts, tears, saliva, milk, feces, sperm, cells or cell cultures, serum, leukocyte fractions, smears, tissue samples of all kinds, embryos, etc. The term also includes both a mixture of the above-mentioned samples such as whole human blood containing a cell. The term “biological sample” also includes untreated or pretreated (or pre-processed) biological samples.

A “biological sample” can contain at least one cell or a plurality of cells from a subject. In some embodiments, the biological sample can contain one or more somatic cells from a subject. In other embodiments, the biological sample can contain one or more germ cells from a subject. In other embodiments, the biological sample can contain one or more stem cells from a subject.

In one embodiment, the biological sample can contain one or more cells from a subject's biological fluid sample. Examples of biological fluids include, but are not limited to, saliva, bone marrow, blood, serum, plasma, urine, sputum, cerebrospinal fluid, an aspirate, tears, and any combinations thereof.

For example, the biological sample can contain one or more circulating tumor cells from a subject's blood (including whole blood, serum, cord blood, and plasma). In some embodiments, the biological sample can contain at least one type of blood cells (e.g., red blood cells, white blood cells, platelets).

In one embodiment, the biological sample can contain one or more cells derived from any tissue of a subject, e.g., a tissue of a normal healthy subject or a tissue suspected of being at risk of, or being afflicted with a given stage of a disease or a disorder. Non-limiting examples of a tissue can include, but are not limited to, breast, pancreas, blood, prostate, colon, lung, skin, brain, ovary, kidney, oral cavity, throat, liver, and any combinations thereof. In some embodiments, the tissue can be obtained from a resection, biopsy, or core needle biopsy. In addition, fine needle aspirate samples can be used. Samples can be either paraffin-embedded or frozen tissue.

The biological sample can be obtained by removing a sample of cells from a subject, but can also be accomplished by using previously isolated cells (e.g. isolated by another person). In addition, the biological sample can be freshly collected or a previously collected sample.

In some embodiments, the biological sample is a frozen biological sample, e.g., a frozen tissue or fluid sample such as urine, blood, serum or plasma. The frozen sample can be thawed before employing methods, assays and systems described herein. After thawing, a frozen sample can be centrifuged before being subjected to methods, assays and systems described herein.

In some embodiments, a biological sample can be a nucleic acid product derived from a tissue (e.g., fresh/frozen and paraffin-embedded) or a fluid sample (e.g., blood) of a subject or cultured cells. The nucleic acid product can include DNA, RNA, mRNA, miRNA, piRNA, siRNA, snRNA, short RNA molecules described herein, and any combinations thereof. In some embodiments, the nucleic acid product can comprise mRNA and short RNA molecules described herein, and any combinations thereof. In one embodiment, the nucleic acid can include short RNA molecules.

In some embodiments, a biological sample can include RNA isolated from a tissue (e.g., fresh or frozen or paraffin-embedded) or a fluid sample (e.g., blood) of a subject or cultured cells. Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In one embodiment, a biological sample can include RNA isolated from a tissue (e.g., fresh or frozen or paraffin-embedded) by any known methods in the art. When the RNA sample is deemed to be of good quality (according to one of skill in the art), the sample can be subjected to further treatment, following recommended instructions as provided by various commercial RNA preparation kits available for RNA sequencing (e.g., the kits from Life Technologies). Depending on the length of RNA molecules of interest, in some embodiments, the RNA sample can be subjected to short RNA sequencing. In other embodiments, the RNA sample can be subjected to long RNA sequencing.

In some embodiments, a biological sample can be an enriched RNA fraction derived from a tissue (e.g., fresh/frozen and paraffin-embedded) or a fluid sample (e.g., blood) of a subject or cultured cells, e.g., an RNA fraction enriched for non-coding RNAs. This can be achieved by, for example, by removing mRNAs by use of affinity purification, e.g., using an oligodT column or any other art-recognized methods such as using commercial small RNA isolation kits.

In some embodiments, a biological sample can be a nucleic acid product or an RNA fraction amplified after polymerase chain reaction (PCR) or after reverse transcription-PCR. The nucleic acid product can include DNA (e.g., cDNA), RNA and mRNA and can be isolated from a particular biological sample using any of a number of procedures, which are well known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

In some embodiments, the biological sample can be treated with a chemical and/or biological reagent. Chemical and/or biological reagents can be employed to protect and/or maintain the stability of the sample, including biomolecules (e.g., nucleic acids) therein, during processing. One exemplary reagent is an RNase inhibitor or RNA stabilizer, which is generally used to protect or maintain the stability of RNA during processing. In addition, or alternatively, chemical and/or biological reagents can be employed to release nucleic acid (e.g., short RNA molecules) from the biological sample.

The skilled artisan is well aware of methods and processes appropriate for pre-processing of biological samples required for determination of nucleic acid including short RNA molecules as described herein.

In some embodiments, the biological sample can be a sample derived or obtained from a normal healthy subject. In some embodiments, the biological sample can be a sample derived or obtained from a subject who is diagnosed with a disease or disorder, e.g., a condition afflicting a tissue. In other embodiments, the biological sample can be derived or obtained from a subject who has or is suspected of having a disease or disorder, e.g., a condition afflicting a tissue, or who is suspected of having a risk of developing a disease or disorder, e.g., a condition afflicting a tissue. In some embodiments, the biological sample can be obtained from a subject who has or is suspected of having cancer, or who is suspected of having a risk of developing cancer. In one embodiment, the biological sample can be obtained from a subject who has or is suspected of having breast cancer, or who is suspected of having a risk of breast cancer. In another embodiment, the biological sample can be obtained from a subject who has or is suspected of having pancreatic cancer, or who is suspected of having a risk of pancreatic cancer.

In some embodiments, the biological sample can be obtained from a subject who is being treated for the disease or disorder, e.g., but not limited to, cancer such as breast cancer or pancreatic cancer. In other embodiments, the biological sample can be obtained from a subject whose previously-treated disease or disorder, e.g., but not limited to, cancer such as breast cancer or pancreatic cancer, is in remission. In other embodiments, the biological sample can be obtained from a subject who has a recurrence of a previously-treated disease or disorder, e.g., but not limited to, cancer such as breast cancer or pancreatic cancer.

As used herein, a “subject” can mean a human, an animal, or a plant. Examples of subjects include primates (e.g., humans, and monkeys). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, and avian species, e.g., chicken, emu, ostrich. Plants include but are not limited to food crops, flowering plants, and grasses. A patient or a subject includes any subset of the foregoing, e.g., all of the above, or includes one or more groups or species such as humans, primates or rodents. In certain embodiments of the aspects described herein, the subject is a mammal, e.g., a primate, e.g., a human. The terms, “patient” and “subject” are used interchangeably herein. A subject can be male or female. The term “patient” and “subject” does not denote a particular age. Thus, any mammalian subjects from adult to newborn subjects, as well as fetuses, are intended to be covered. Any vertebrate or invertebrate subjects of any age as well as plants are also intended to be covered.

In one embodiment, the subject or patient is a mammal. The mammal can be a human, non-human primate, mouse, rat, dog, cat, horse, or cow, but are not limited to these examples. In one embodiment, the subject is a human being. In another embodiment, the subject can be a domesticated animal and/or pet.

In another embodiment, the subject can be a food crop, a flowering plant or a grass.

Embodiments of the Various Aspects Described Herein can be Illustrated by the Following Numbered Paragraphs.
1. A method of determining whether a subject has, or is at risk of developing, or is at a given stage of a condition afflicting a tissue of interest, comprising the measuring in a biological sample from the tissue of interest of the expression level of one or more short RNA sequences originating from (a) one or more exons of one or more protein-coding genes; and/or (b) one or more segments of one or more non-coding transcripts, wherein the alteration of the level of said one or more short RNA sequences as compared to the level of the same one or more short RNA sequences in a reference sample is indicative of the subject either having, or being at risk of developing, or is at a given stage of the condition.
2. The method of paragraph 1, wherein the reference sample represents a normal condition of the tissue.
3. The method of paragraph 1 or 2, wherein the reference sample represents a recognizable stage of an abnormal condition of the tissue.
4. The method of any of paragraphs 1-3, wherein the tissue of interest is breast.
5. The method of any of paragraphs 1-3, wherein the tissue of interest is pancreas.
6. The method of any of paragraphs 1-3, wherein the tissue of interest is blood.
7. The method of any of paragraphs 1-3, wherein the tissue of interest is prostate.
8. The method of any of paragraphs 1-3, wherein the tissue of interest is colon.
9. The method of any of paragraphs 1-3, wherein the tissue of interest is lung.
10. The method of any of paragraphs 1-3, wherein the tissue of interest is skin.
11. The method of any of paragraphs 1-3, wherein the tissue of interest is brain.
12. The method of any of paragraphs 1-3, wherein the tissue of interest is liver.
13. The method of any of paragraphs 1-3, wherein the tissue of interest is ovary.
14. The method of any of paragraphs 1-3, wherein the tissue of interest is bone marrow.
15. The method of any of paragraphs 1-3, wherein the tissue of interest is muscle.
16. The method of paragraph 4, wherein the condition of interest is ductal in situ carcinoma (breast carcinoma).
17. The method of paragraph 4, wherein the condition of interest is invasive breast cancer.
18. The method of paragraph 5, wherein the condition of interest is early stage pancreatic cancer.
19. The method of paragraph 5, wherein the condition of interest is late stage pancreatic cancer.
20. The method of paragraph 16, wherein the protein-coding genes of interest comprise ABCC11, ACTB, ACTG1, AHCY, AHNAK, ANKHD1, APP, ARF1, ASAH1, ATP1A1, ATP1B1, ATP6V0E1, AZGP1, B2M, B4GALT1, BAZ2A, BST2, BTG2, C1orf43, C3orf1, CALM2, CALR, CANX, CCNI, CD151, CD164, CD44, CD46, CD59, CD74, CEACAM6, CIRBP, CLIC6, COL1A2, COL6A1, COL6A3, COMMD3, COX7A2, CSDE1, CSRP1, CST3, CTNND1, CTSB, CXCL13, CYBRD1, DCN, DDX17, DDX5, DSP, DUSP4, EEF2, EFHD1, EHF, EIF4EBP3, EIF4G2, ELOVL5, ERBB2, ERBB3, ESR1, FASN, FAT1, FLNB, FMOD, FN1, FOXA1, FTL, GAPDH, GATA3, GDI2, GJA1, GLUL, HDLBP, HIST1H1B, HIST1H2AC, HIST1H3D, HIST1H4H, HNRNPF, HSP90AB1, IFI6, IGFBP4, IGHG4, ITGB4, JUP, KIAA0100, KIAA1522, LAPTM4A, LPHN1, LRBA, LRP2, MAGED2, MDH1, MED13L, MKNK2, MLL5, MLPH, MT-CO2, MUC1, MYB, MYH9, MYL6, NCL, NDUFA2, NET1, NF1, NME1, NUCKS1, P4HB, PACS2, PCBP2, PDCD4, PDIA3, PDLIM1, PDXDC1, PEG10, PFN1, PGR, PI15, PNRC1, PPDPF, PSMD5, PTPRF, QDPR, RARG, RBM39, RHOA, RHOB, RNF41, RPL15, S100A16, SEC11A, SERPINA1, SERPINA3, SFRP2, SH3BGRL, SIAH2, SLC25A6, SLC26A2, SLC38A1, SLC39A6, SLC7A2, SMG5, SREBF2, SRRM2, SSR2, STEAP1, STOM, TAGLN2, TAT, TFF3, TGOLN2, THAP4, TMBIM6, TMC5, TMED2, TMED5, TMEM59, TMEM66, TOB1, TPT1, TRPS1, TSPAN1, TTC39A, TUFM, TXNIP, UBN1, UBXN4, UFC1, UGDH, UNC13B, VIM, WAPAL, WIPI1, WNK1, XBP1, ZBTB7B and other, and combinations thereof.
21. The method of paragraph 17, wherein the protein coding genes of interest comprise ABCC11, ACTB, ACTG1, ADAR, AFF3, AHCY, AHNAK, ANKHD1, APP, ARF1, ARHGDIB, ASAH1, ATP1A1, ATP1B1, ATP6V0E1, AZGP1, B2M, B4GALT1, BAZ2A, BST2, BTG2, C1orf43, C5orf45, CALM2, CALR, CANX, CCNI, CD151, CD164, CD44, CD46, CD59, CD74, CD81, CEACAM6, CELSR1, CELSR2, CEP350, CILP, CIRBP, CLDN4, CLIC6, COL1A2, COL3A1, COL6A3, COMMD3, COX7A2, CSDE1, CSRP1, CTNNA1, CTNNB1, CTSD, CXCL13, CYBRD1, DBI, DCN, DDX17, DDX5, DSP, DUSP4, EEF2, EFHD1, EHF, EIF4EBP3, EIF4G2, ELF3, ELOVL5, EPRS, ERBB2, ERBB3, ESR1, FASN, FHL2, FLNB, FMOD, FOXA1, FTH1, GAPDH, GATA3, GDI2, GJA1, GLUL, GNAS, GNB2L1, GSTK1, HDLBP, HIST1H1C, HIST1H1D, HIST1H1E, HIST1H2AC, HIST1H2AE, HIST1H2BC, HIST1H2BD, HIST1H3D, HIST1H4B, HIST1H4D, HIST1H4H, HIST2H2AB, HIST2H2AC, HIST4H4, HNRNPF, HSP90AA1, HSP90AB1, IFI6, IGFBP4, IGHG1, IGHG4, IGKC, JTB, JUP, KIAA0100, KIAA1522, KRT19, LAPTM4A, LMNA, LONP2, LPHN1, LRBA, MAGED2, MCL1, MDH1, MED13L, MGP, MKNK2, MLL5, MLPH, MPZL1, MT-CO2, MT-CYB, MUC1, MYB, MYH9, MYST3, NCL, NDUFA2, NDUFB5, NET1, NF1, NFIB, NME1, NUCKS1, OAZ1, P4HB, PACS2, PCBP2, PDCD4, PDIA3, PDLIM1, PDXDC1, PEG10, PFN1, PGR, PHB2, PI15, PNRC1, PPDPF, PRICKLE4, PSAP, PTPRF, QDPR, RARG, RBM39, RHOA, RHOB, RNF41, RPL13AP20, RPL15, RPL17, RPL4, RPLP2, RPS2, S100A11, S100A14, S100A16, S100A9, SAT1, SEMA3C, SERPINA1, SERPINA3, SF3B1, SGK3, SH3BGRL, SIAH2, SLC25A3, SLC25A6, SLC26A2, SLC38A1, SLC39A6, SLC7A2, SMG5, SPARC, SPTBN1, SREBF2, SRRM2, SSR2, STEAP1, STOM, TAGLN2, TAT, TBC1D16, TFF3, TGOLN2, THAP4, TM9SF2, TMBIM6, TMC5, TMED2, TMEM59, TMEM66, TOB1, TOMM6, TPT1, TRPS1, TSPAN1, TTC39A, TUFM, TXNIP, UCK2, UFC1, UGDH, UNC13B, WIPI1, WNK1, XBP1, ZBTB7B, ZNF207 and other, and combinations thereof.
22. The method of paragraph 18, wherein the protein coding genes of interest comprise ACTG1, ALB, AMY2B, C7, CEL, CELA3A, CLPS, COL3A1, CPA1, CPA2, CPB1, CTRB1, CTRB2, CUZD1, EEF2, GANAB, GATM, GP2, HDLBP, KHDRBS1, KLK1, KRT7, OLFM4, P4HB, PLA2G1B, PPDPF, PRSS1, PRSS3, REG1A, REG1B, REG3A, RNASE1, RPL8, SPINK1, SYCN, UNC13B and other, and combinations thereof
23. The method of paragraph 19, wherein the protein coding genes of interest comprise ACTB, ANXA2, ANXA5, APOE, ATP6VOC, C1QA, C1QB, C1QC, CIS, CALR, CCNI, CD14, CD44, CD59, CD68, COL1A2, COL6A3, CTSB, CTSC, EEF2, F13A1, FLNA, FN1, GLUL, GPNMB, GPX1, HIST1H2BD, IGFBP4, IGHM, IGKC, ISG15, LAMB3, LAPTM5, LGALS3BP, METTL7A, MMP11, MMP14, MT-CO2, MT-CYB, MYH9, OAZ1, P4HB, PLEC, PSAP, RNASE1, RPN1, SAT1, SERPINA1, SERPING1, SLC40A1, SLCO2B1, SPP1, SRGN, TGM2, TGOLN2, TIMP2, TXNIP, VSIG4, ZYX and other, and combinations thereof
24. The method of any of paragraphs 1-23, wherein the short RNAs of interest are segments of the exons of the one or more genes of interest, and/or segments of said one or more non-coding transcripts.
25. A method of determining a given state of a cell or a tissue, the method comprising detecting in a biological sample the presence or absence of a short RNA sequence originating from an exon of at least one protein-coding gene, and/or from a segment of at least one non-coding transcript.
26. A method of identifying an origin and/or type of a cell or a tissue, the method comprising detecting in a biological sample the presence or absence of a short RNA sequence originating from an exon of at least one protein-coding gene, and/or from a segment of at least one non-coding transcript.
27. A method of distinguishing an origin and/or type of a first tissue from a second tissue, the method comprising detecting in a first biological sample the presence or absence of a short RNA sequence originating from an exon of at least one protein-coding gene, and/or from a segment of at least one non-coding transcript, wherein a difference in an expression level of the short RNA sequence between the first and the second biological sample is indicative of the first tissue having an origin and/or type different from that of the second tissue.
28. The method of paragraph 27, further comprising detecting in a second biological sample the presence or absence of the short RNA sequence.
29. A method of determining whether a subject has, or is at risk of developing, or is at a given stage of a condition afflicting a tissue of interest, the method comprising detecting in a biological sample the presence or absence of a short RNA sequence originating from an exon of at least one protein-coding gene, and/or from a segment of at least one non-coding transcript.
30. The method of any of paragraphs 25-29, wherein said detecting the presence or absence of the short RNA sequence includes measuring an expression level of the short RNA sequence in the biological sample.
31. The method of paragraph 30, further comprising comparing with a reference sample the expression level of the short RNA sequence in the biological sample, wherein an alteration of the expression level of the short RNA sequence in the biological sample as compared to the reference sample is indicative of the cell or tissue represented by the biological sample having a state, an origin and/or a type different from that of the reference sample.
32. The method of paragraph 30, further comprising comparing with a reference sample the expression level of the short RNA sequence in the biological sample, wherein an alteration of the expression level of the short RNA sequence in the biological sample as compared to the reference sample is indicative of the subject either having, or being at risk of developing, or is at a given stage of the condition.
33. The method of any of paragraphs 25-32, wherein said detecting the presence or absence of the short RNA sequence includes identifying an originating location of the short RNA sequence from the exon, or from the non-coding transcript.
34. The method of paragraph 33, further comprising comparing with a reference sample the originating location of the short RNA sequence in the biological sample, wherein a discrepancy in the originating location of the short RNA sequence in the biological sample from the reference sample is indicative of the cell or tissue represented by the biological sample having a state, an origin and/or a type different from that of the reference sample.
35. The method of paragraph 33, further comprising comparing with a reference sample the originating location of the short RNA sequence in the biological sample, wherein a discrepancy in the originating location of the short RNA sequence in the biological sample from the reference sample is indicative of the subject either having, or being at risk of developing, or is at a given state of a condition.
36. The method of any of paragraphs 25-35, wherein the method comprises detecting in the biological sample the presence or absence of a plurality of short RNA sequences originating from an exon of at least one protein-coding gene, and/or from a segment of at least one non-coding transcript.
37. The method of paragraph 36, wherein the plurality of short RNA sequences are originated from more than one exons of at least one protein-coding gene, and/or from more than one segments of at least one non-coding transcript.
38. The method of any of paragraphs 25-37, wherein the short RNA sequence is at least a segment of the exon of said at least one protein-coding gene or a segment of said at least one non-coding transcript.
39. The method of paragraph 38, wherein the segment has a length of about 20 nucleotides to about 40 nucleotides.
40. The method of paragraph 39, wherein the segment has a length of about 32 nucleotides to about 40 nucleotides.
41. The method of paragraph 40, wherein the segment has a length of about 34 nucleotides.
42. The method of any of paragraphs 31-41, wherein the reference sample represents a normal condition of a cell or tissue.
43. The method of any of paragraphs 31-41, wherein the reference sample represents a recognizable stage of an abnormal condition of a cell or a tissue.
44. The method of any of paragraphs 25-43, wherein the biological sample is one or more cells derived from the tissue of interest.
45. The method of any of paragraphs 25-44, wherein the tissue of interest is breast.
46. The method of any of paragraphs 25-44, wherein the tissue of interest is pancreas.
47. The method of any of paragraphs 25-44, wherein the tissue of interest is blood.
48. The method of any of paragraphs 25-44, wherein the tissue of interest is prostate.
49. The method of any of paragraphs 25-44, wherein the tissue of interest is colon.
50. The method of any of paragraphs 25-44, wherein the tissue of interest is lung.
51. The method of any of paragraphs 25-44, wherein the tissue of interest is skin.
52. The method of any of paragraphs 25-44, wherein the tissue of interest is brain.
53. The method of any of paragraphs 25-44, wherein the tissue of interest is liver.
54. The method of any of paragraphs 25-44, wherein the tissue of interest is ovary.
55. The method of any of paragraphs 25-44, wherein the tissue of interest is bone marrow.
56. The method of any of paragraphs 25-44, wherein the tissue of interest is muscle.
57. The method of any of paragraphs 25-56, wherein the given state or the condition includes cancer.
58. The method of paragraph 57, wherein when the cancer is breast carcinoma, the given stage of the condition is ductal in situ carcinoma.
59. The method of paragraph 58, wherein the protein-coding gene is selected from the group consisting of ABCC11, ACTB, ACTG1, AHCY, AHNAK, ANKHD1, APP, ARF1, ASAH1, ATP1A1, ATP1B1, ATP6V0E1, AZGP1, B2M, B4GALT1, BAZ2A, BST2, BTG2, C1orf43, C3orf1, CALM2, CALR, CANX, CCNI, CD151, CD164, CD44, CD46, CD59, CD74, CEACAM6, CIRBP, CLIC6, COL1A2, COL6A1, COL6A3, COMMD3, COX7A2, CSDE1, CSRP1, CST3, CTNND1, CTSB, CXCL13, CYBRD1, DCN, DDX17, DDX5, DSP, DUSP4, EEF2, EFHD1, EHF, EIF4EBP3, EIF4G2, ELOVL5, ERBB2, ERBB3, ESR1, FASN, FAT1, FLNB, FMOD, FN1, FOXA1, FTL, GAPDH, GATA3, GDI2, GJA1, GLUL, HDLBP, HIST1H1B, HIST1H2AC, HIST1H3D, HIST1H4H, HNRNPF, HSP90AB1, IFI6, IGFBP4, IGHG4, ITGB4, JUP, KIAA0100, KIAA1522, LAPTM4A, LPHN1, LRBA, LRP2, MAGED2, MDH1, MED13L, MKNK2, MLL5, MLPH, MT-CO2, MUC1, MYB, MYH9, MYL6, NCL, NDUFA2, NET1, NF1, NME1, NUCKS1, P4HB, PACS2, PCBP2, PDCD4, PDIA3, PDLIM1, PDXDC1, PEG10, PFN1, PGR, PI15, PNRC1, PPDPF, PSMD5, PTPRF, QDPR, RARG, RBM39, RHOA, RHOB, RNF41, RPL15, S100A16, SEC11A, SERPINA1, SERPINA3, SFRP2, SH3BGRL, SIAH2, SLC25A6, SLC26A2, SLC38A1, SLC39A6, SLC7A2, SMG5, SREBF2, SRRM2, SSR2, STEAP1, STOM, TAGLN2, TAT, TFF3, TGOLN2, THAP4, TMBIM6, TMC5, TMED2, TMED5, TMEM59, TMEM66, TOB1, TPT1, TRPS1, TSPAN1, TTC39A, TUFM, TXNIP, UBN1, UBXN4, UFC1, UGDH, UNC13B, VIM, WAPAL, WIPI1, WNK1, XBP1, ZBTB7B, and any combinations thereof.
60. The method of paragraph 57, wherein when the cancer is breast carcinoma, the given stage of the condition is lobular in situ carcinoma.
61. The method of paragraph 57, wherein when the cancer is breast carcinoma, the given stage of the condition is invasive breast carcinoma.
62. The method of paragraph 61, wherein the protein coding gene is selected from the group consisting of ABCC11, ACTB, ACTG1, ADAR, AFF3, AHCY, AHNAK, ANKHD1, APP, ARF1, ARHGDIB, ASAH1, ATP1A1, ATP1B1, ATP6V0E1, AZGP1, B2M, B4GALT1, BAZ2A, BST2, BTG2, C1orf43, C5orf45, CALM2, CALR, CANX, CCNI, CD151, CD164, CD44, CD46, CD59, CD74, CD81, CEACAM6, CELSR1, CELSR2, CEP350, CILP, CIRBP, CLDN4, CLIC6, COL1A2, COL3A1, COL6A3, COMMD3, COX7A2, CSDE1, CSRP1, CTNNA1, CTNNB1, CTSD, CXCL13, CYBRD1, DBI, DCN, DDX17, DDX5, DSP, DUSP4, EEF2, EFHD1, EHF, EIF4EBP3, EIF4G2, ELF3, ELOVL5, EPRS, ERBB2, ERBB3, ESR1, FASN, FHL2, FLNB, FMOD, FOXA1, FTH1, GAPDH, GATA3, GDI2, GJA1, GLUL, GNAS, GNB2L1, GSTK1, HDLBP, HIST1H1C, HIST1H1D, HIST1H1E, HIST1H2AC, HIST1H2AE, HIST1H2BC, HIST1H2BD, HIST1H3D, HIST1H4B, HIST1H4D, HIST1H4H, HIST2H2AB, HIST2H2AC, HIST4H4, HNRNPF, HSP90AA1, HSP90AB1, IFI6, IGFBP4, IGHG1, IGHG4, IGKC, JTB, JUP, KIAA0100, KIAA1522, KRT19, LAPTM4A, LMNA, LONP2, LPHN1, LRBA, MAGED2, MCL1, MDH1, MED13L, MGP, MKNK2, MLL5, MLPH, MPZL1, MT-CO2, MT-CYB, MUC1, MYB, MYH9, MYST3, NCL, NDUFA2, NDUFB5, NET1, NF1, NFIB, NME1, NUCKS1, OAZ1, P4HB, PACS2, PCBP2, PDCD4, PDIA3, PDLIM1, PDXDC1, PEG10, PFN1, PGR, PHB2, PI15, PNRC1, PPDPF, PRICKLE4, PSAP, PTPRF, QDPR, RARG, RBM39, RHOA, RHOB, RNF41, RPL13AP20, RPL15, RPL17, RPL4, RPLP2, RPS2, S100A11, S100A14, S100A16, S100A9, SAT1, SEMA3C, SERPINA1, SERPINA3, SF3B1, SGK3, SH3BGRL, SIAH2, SLC25A3, SLC25A6, SLC26A2, SLC38A1, SLC39A6, SLC7A2, SMG5, SPARC, SPTBN1, SREBF2, SRRM2, SSR2, STEAP1, STOM, TAGLN2, TAT, TBC1D16, TFF3, TGOLN2, THAP4, TM9SF2, TMBIM6, TMC5, TMED2, TMEM59, TMEM66, TOB1, TOMM6, TPT1, TRPS1, TSPAN1, TTC39A, TUFM, TXNIP, UCK2, UFC1, UGDH, UNC13B, WIPI1, WNK1, XBP1, ZBTB7B, ZNF207, and any combinations thereof.
63. The method of paragraph 57, wherein when the cancer is pancreatic cancer, the given stage of the condition is early stage pancreatic cancer
64. The method of paragraph 63, wherein the protein coding gene is selected from the group consisting of ACTG1, ALB, AMY2B, C7, CEL, CELA3A, CLPS, COL3A1, CPA1, CPA2, CPB1, CTRB1, CTRB2, CUZD1, EEF2, GANAB, GATM, GP2, HDLBP, KHDRBS1, KLK1, KRT7, OLFM4, P4HB, PLA2G1B, PPDPF, PRSS1, PRSS3, REG1A, REG1B, REG3A, RNASE1, RPL8, SPINK1, SYCN, UNC13B, and any combinations thereof
65. The method of paragraph 57, wherein when the cancer is pancreatic cancer, the given stage of the condition is late stage pancreatic cancer.
66. The method of paragraph 65, wherein the protein coding gene is selected from the group consisting of ACTB, ANXA2, ANXA5, APOE, ATP6VOC, C1QA, C1QB, C1QC, CIS, CALR, CCNI, CD14, CD44, CD59, CD68, COL1A2, COL6A3, CTSB, CTSC, EEF2, F13A1, FLNA, FN1, GLUL, GPNMB, GPX1, HIST1H2BD, IGFBP4, IGHM, IGKC, ISG15, LAMB3, LAPTM5, LGALS3BP, METTL7A, MMP11, MMP14, MT-CO2, MT-CYB, MYH9, OAZ1, P4HB, PLEC, PSAP, RNASE1, RPN1, SAT1, SERPINA1, SERPING1, SLC40A1, SLCO2B1, SPP1, SRGN, TGM2, TGOLN2, TIMP2, TXNIP, VSIG4, ZYX, and any combinations thereof
67. The method of any of paragraphs 25-66, wherein when the protein-coding gene is ELOVL5, at least a portion of the short RNA sequence is selected from the group consisting of SEQ ID NO: 1 to SEQ ID NO: 80, or a fragment thereof.
68. The method of any of paragraphs 25-67, wherein the exon includes an untranslated region of the protein-coding gene.
69. The method of any of paragraphs 25-68, wherein the short RNA sequence has an overlapping region with a pyknon.
70. The method of any of paragraphs 25-69, further comprising administering or prescribing a treatment to the subject determined to have, or is at risk of developing, or is at a given stage of the condition.
71. A system for analyzing a biological sample comprising:
   a) a determination module configured to receive a biological sample and to determine sequence information, wherein the sequence information comprises a sequence of a short RNA molecule originating from an exon of at least one protein-coding gene and/or from a segment of at least one non-coding transcript;
   b) a storage device configured to store sequence information from the determination module;
   c) a comparison module adapted to compare the sequence information stored on the storage device with reference data, and to provide a comparison result, wherein the comparison result identifies the presence or absence of the short RNA molecule, wherein a discrepancy in an expression level or in an originating location of the short RNA molecule from the reference data is indicative of the biological sample having an increased likelihood of having or being at a cellular or tissue state different from a state represented by the reference data; and
   d) a display module for displaying a content based in part on the comparison result for the user, wherein the content is a signal indicative of a subject having, or being at risk of developing, or being at a given stage of a disease or disorder, or a signal indicative of lack of a disease or disorder.
72. A computer-readable physical medium having computer readable instructions recorded thereon to define software modules including a comparison module and a display module for implementing a method on a computer, said method comprising:
   a) comparing with the comparison module the data stored on a storage device with reference data to provide a comparison result, wherein the comparison result the comparison result identifies the presence or absence of the short RNA molecule, wherein a discrepancy in an expression level or in an originating location of the short RNA molecule from the reference data is indicative of the biological sample having an increased likelihood of having or being at a cellular or tissue state different from a state represented by the reference data; and
   b) a display module for displaying a content based in part on the comparison result for the user, wherein the content is a signal indicative of a subject having, or being at risk of developing, or being at a given stage of a disease or disorder, or a signal indicative of lack of a disease or disorder.

### Some Selected Definitions

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to describe the present invention, in connection with percentages means ±1%.

In one respect, the present invention relates to the herein described compositions, methods, and respective component(s) thereof, as essential to the invention, yet open to the inclusion of unspecified elements, essential or not (“comprising”). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventor is not entitled to antedate such disclosure by virtue of prior invention or for any other reason. All statements as to the date or representation as to the contents of these documents is based on the information available to the applicants and does not constitute any admission as to the correctness of the dates or contents of these documents.

The term “statistically significant” or “significantly” or “significant” refers to statistical significance and generally means a one standard deviation (1 SD) above or below a reference level. The term refers to statistical evidence that there is a difference. It is defined as the probability of making a decision to reject the null hypothesis when the null hypothesis is actually true. The decision is often made using the p-value.

The term “deep sequencing” as used herein generally refers to next- or higher-generation sequencing known to a skilled artisan.

The term “nucleic acid” is well known in the art. A “nucleic acid” as used herein will generally refer to a molecule (i.e., strand) of DNA, RNA or a derivative or analog thereof, comprising a nucleobase. A nucleobase includes, for example, a naturally occurring purine or pyrimidine base found in DNA (e.g. an adenine “A,” a guanine “G,” a thymine “T” or a cytosine “C”) or RNA (e.g. an A, a G, an uracil “U” or a C). The term “nucleic acid” encompasses the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length.

The term “gene” has traditionally been used to refer to the segment of DNA involved in producing a polypeptide chain. In higher organisms, the region of DNA corresponding to a gene comprises a combination of sequences that are removed during splicing (introns) and sequences (exons) that are combined into the messenger RNA (mRNA) from which the amino acid product will be obtained following mRNA translation. Segments of exons, and on occasion entire exons, can remain untranslated and are referred to as “untranslated regions” or UTRs. In some embodiments, the term “gene” can also encompass any identifiable molecule that is transcribed from a DNA sequence and independently of whether it will give rise to an amino acid sequence. In other words, the term “gene” can be used to refer to both “protein-coding” transcripts (and their respective DNA sequences) and “non-protein-coding” transcripts (and their respective DNA sequences), and this expanded definition is used herein.

As used herein, the term “intron” refers to a nucleotide sequence in the primary unspliced transcript of a DNA sequence that separates two exons. The art traditionally used the term “intron” in the context of nascent unspliced mRNAs to refer to the sequences between exons that were removed during splicing of the mRNA and prior to translation by the ribosome. Recently, the terms “intron” and “exon” have been expanded to include non-coding transcripts, i.e., transcripts that do not lead to an amino acid product. For example, in the case of nascent mRNAs, non-coding sequences can be transcribed from genomic DNA and form a precursor transcript that can be processed and spliced into one or more shorter “product” transcripts: those segments of the precursor transcript that are part of the “product” are referred to as “exons” and the intervening sequences separating them are referred to as ‘introns.” In some embodiments, this expanded definition is used herein. In some embodiments, the terms “intron” and “exon” refer to non-coding transcripts, i.e., transcript that do not lead to an amino acid product.

The term “non-coding” refers to sequences of nucleic acid molecules that cannot be translated in a sequence-specific manner to produce into a particular polypeptide or peptide. In some embodiments, the term “non-coding” in reference to RNA can refer to a RNA sequence that is not translated in a sequence-specific manner to produce a particular polypeptide or peptide. In some embodiments, a non-coding RNA can comprise a sequence corresponding to a fragment of a protein-coding region, but which is not translated into a functional peptide or protein when it forms part of a non-coding RNA. Non-coding sequences include but are not limited to introns or parts thereof, promoter regions or parts thereof, 3′ untranslated regions (3′ UTR) or parts thereof, 5′ untranslated regions (5′ UTR) or parts thereof, as well as intergenic regions. In general, a 3′ or 5′ untranslated region is part of or spans one or more exons.

The term “coding region” or “protein-coding region” as used herein, refers to a portion of the nucleic acid sequence, which is transcribed and translated in a sequence-specific manner to produce a particular polypeptide or protein when placed under the control of appropriate regulatory sequences and appropriate molecular machinery. The coding region of a protein-coding gene is said to encode one, or more, such polypeptide or protein.

The term “oligonucleotide,” as used herein refers to primers and probes, and is defined as a nucleic acid molecule, or its sequence representation, comprised of at least two or more ribo- or deoxyribonucleotides. The exact size of the oligonucleotide will depend on various factors and on the particular application and use of the oligonucleotide. The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and the method used. For example, for diagnostic applications, depending on the complexity of the target sequence, an oligonucleotide probe typically contains 15-25 or more nucleotides, although it may contain fewer nucleotides. The probes as disclosed herein are selected to be “substantially complementary” to different strands of a particular target nucleic acid sequence. This means that the probes must be sufficiently complementary so as to be able to “specifically hybridize” or anneal with their respective target strands. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarily with the sequence of the target nucleic acid to anneal therewith specifically.

In the context of this disclosure, the term “probe” refers to a molecule that can detectably distinguish among target molecules differing in sequence composition and also in structure (e.g. nucleic acid or protein sequence). Detection can be accomplished in a variety of different ways depending on the type of probe used and the type of target molecule. Thus, for example, detection may be based on discrimination on detection of specific binding. Examples of such specific binding include antibody binding and nucleic acid, antibody binding to protein, nucleic acid binding to nucleic acid, or aptamer binding to protein or nucleic acid. Thus, for example, probes can include enzyme substrates, antibodies and antibody fragments, and preferably nucleic acid hybridization probes.

The term “specifically hybridize” refers to the association between two single-stranded nucleic acid molecules of sufficient complementary sequence to permit such hybridization under pre-determined conditions generally used in the art (sometimes the sequences are referred to as “substantially complementary”). In particular, the term specifically hybridize also refers to hybridization of an oligonucleotide with a substantially complementary sequence as compared to non-complementary sequence.

The term “specifically” as used herein with reference to a probe which is used to specifically detect a given sequence of contiguous nucleotides, refers to a probe that identifies the particular sequence based on preferential hybridization to the sequence under consideration stringent hybridization conditions and/or on exclusive amplification or replication of molecules of interest.

The term “specifically” as used herein with reference to a probe which is used to specifically detect a sequence difference, refers to a probe that identifies a particular sequence difference based on exclusive hybridization to the sequence difference under stringent hybridization conditions and/or on exclusive amplification or replication of the sequence difference.

In its broadest sense, the term “substantially” as used herein in respect to “substantially complementary”, or when used herein with respect to a nucleotide sequence in relation to a reference or a target nucleotide sequence, means a nucleotide sequence having a percentage of identity between the substantially complementary nucleotide sequence and the exact complementary sequence of said reference or target nucleotide sequence of at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of at least 10 nucleotides, or at least 11, 12, 13, 14, 15, 16, 17, 18, 19, 20, 21, 22 or up to 50 nucleotides of the entire length of the nucleic acid sequence to said reference sequence (if not specified otherwise below). Sequence comparisons can be carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above), or any of the tools that have been used for this purpose by the skilled artisan. A nucleotide sequence “substantially complementary” to a reference nucleotide sequence hybridizes to the reference nucleotide sequence under low stringency conditions, preferably medium stringency conditions, most preferably high stringency conditions.

In its broadest sense, the term “substantially identical,” when used herein with respect to a nucleotide sequence, means a nucleotide sequence corresponding to a reference or target nucleotide sequence, wherein the percentage of identity between the substantially identical nucleotide sequence and the reference or target nucleotide sequence is at least 60%, at least 70%, at least 80% or 85%, at least 90%, at least 93%, at least 95% or 96%, at least 97% or 98%, at least 99% or 100% (the later being equivalent to the term “identical” in this context). For example, identity is assessed over a length of 10-40 nucleotides, such as at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to 50 nucleotides of a nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J MoI. Biol. 48: 443-453; as defined above), or similar tools, as mentioned above. A nucleotide sequence “substantially identical” to a reference nucleotide sequence hybridizes to the exact complementary sequence of the reference nucleotide sequence (i.e. its corresponding strand in a double-stranded molecule) under low stringency conditions, preferably medium stringency conditions, most preferably high stringency conditions (as defined above). Homologues of a specific nucleotide sequence include nucleotide sequences that is at least 24% identical, at least 35% identical, at least 50% identical, at least 65% identical to the reference sequence, as measured using the parameters described above, wherein the molecule represented by the homologous sequence is considered to have the same biological activity as the molecule encoded by the specific nucleotide sequence. The term “substantially non-identical” refers to a nucleotide sequence that does not hybridize to the nucleic acid sequence under stringent conditions.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length, but can be longer as needed. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “complementary” as used herein refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine (for DNA) or uracil (for RNA). Similarly, it is known that a cytosine residue of a first nucleic acid strand is capable of base pairing with a residue of a second nucleic acid strand which is anti-parallel to the first strand if the residue is guanine A cytosine residue of a first nucleic acid strand is also capable of base pairing with a residue of a second nucleic acid strand which is anti-parallel to the first strand if the residue is uracil—such interactions are referred to as “non-Watson-Crick” or “G:U wobbles.” A first region of a nucleic acid is complementary to a second region of the same or a different nucleic acid if at least one nucleotide residue of the first region is capable of base pairing with a residue of the second region, when the two regions are arranged in an anti-parallel fashion. Preferably, the first region comprises a first portion and the second region comprises a second portion, whereby, when the first and second portions are arranged in an anti-parallel fashion, such that at least about 50%, and preferably at least about 75%, at least about 90%, or at least about 95% or at least 100% of the nucleotide residues of the first portion are capable of base pairing with nucleotide residues in the second portion. More preferably, all nucleotide residues of the first portion are capable of base pairing with nucleotide residues in the second portion. A first region of a nucleic acid is “near-complementary” to a second region of the same or a different nucleic acid if, at least one nucleotide residue of the first region is capable of base pairing with a residue of the second region, when the two regions are arranged in an anti-parallel fashion, and not all of the nucleotides of the two regions are base-paired. Such interactions are exemplified by heteroduplexes of miRNAs with mRNAs where the typical interaction between the two molecules is effected by only a subset of the residues spanning each region. Additionally, the two interacting regions need not have the same length.

### EXAMPLES

The examples presented herein relate to methods of identifying genes that can produce short RNAs out of their exon(s), which can be used as biomarkers for diagnosis or prognosis of a disease, e.g., cancer, or disorder. Methods of determining a given stage of a disease or disorder afflicting a tissue, e.g., a given stage of a cancer, based on the presence and/or amount levels of short RNAs originating from one or more exons of a gene in a tissue sample are also provided herein. Even though the presented examples use genomic regions and sequences that are associated with protein-coding transcripts (protein-coding exonic regions and/or untranslated exonic regions of mRNAs), it should be understood that the observations made herein readily extend to non-protein-coding RNAs (i.e., non-coding RNAs) where the presence or absence of one or more short RNAs originating in a region that normally gives rise to a long (non-coding) transcript would be indicative of the emergence of a new state for the tissue at hand: one such example is described below and pertains to a non-coding transcript, e.g., MALAT1 (also known as NEAT2).

### Example 1

### Exemplary Methods for Identification of Genes that Produce Short RNAs Out of their Exons

Human samples from breast and pancreas (both normal and diseased/abnormal) were obtained for deep sequencing, e.g., next generation sequencing. The NGS focused on generating a profile of the short RNAs contained in those samples. The NGS was carried out on a Life Technologies SOLiD 3+ platform. For each sample, a large dataset that contained the sequenced reads in Life Technologies' “colorspace” format was obtained. Using a read mapping program, e.g., Burrows-Wheeler Alignment tool as described in Li and Durbin (2009) Bioinformatics 25(14): 1754-1760, the sequenced reads were mapped on the assembly of the human genome (e.g., using hg19 which can be assessed at hgdownload.cse.ucsc.edu/downloads.html#human. If a sequenced read mapped at multiple locations of the genome, then all instances of the read were discarded. This ensured that the genomic locations that gave rise to sequenced RNA reads could be unambiguously determined.

Each sequenced read set gave rise to a genomic map that showed the provenance of the short RNA molecules that were deep-sequenced from the corresponding RNA sample (breast normal/diseased, pancreas normal/diseased). There were a total of 8 such genomic maps (4 from the breast samples and 4 from the pancreas samples). The genomic maps could be visualized with any genomic browser known in the art, e.g., the Univ. of California at Santa Cruz Genome Browser (which can be assessed at genome.ucsc.edu/cgi-bin/hgGateway).

Based on an analysis of these genomic maps, it was unexpectedly determined that some protein coding genes gave rise to “short” RNAs out of some of their exons and such short RNAs were originated from those exons in a state-specific and tissue-specific manner. Generally, it is known in the art that the exons of a protein-coding region make up a transcript (i.e., the mRNA), which is translated by the ribosome into an amino acid sequence. More importantly, the apparent dependence of the short RNAs on tissue and on tissue-state indicates that the short RNAs can be used to determine the state of a tissue (diseased or abnormal vs. normal), e.g., by detecting the presence or absence and/or measuring levels of amount of these short RNAs.

By analyzing short RNA profiles, it was determined that a gene (e.g., ELOVL5 described below) exhibited abundant production of such short RNAs out of its exons in breast cancer tissue samples. Thus, a specific program was used to analyze all of the genomic maps obtained from the profiling of short RNAs in the breast and cancer samples and across the entire genome. In the program, each genomic map was intersected with the coordinates of the exons of the known protein-coding genes, which generated a collection of “islands” that (a) overlapped protein-coding exons and (b) generated short RNAs. Then, by sliding a window across each of these islands, it was determined whether a significant fraction of the window's span gave rise to short RNAs and whether the change in amount of these short RNAs between two tissue samples (e.g., normal breast vs. diseased breast) exceeded a certain threshold. This allowed identifying a protein-coding gene that satisfied these requirements.

For example, one of the regions that were identified was located on chromosome 6 and corresponded to the 3′ UTR region of a gene known as ELOVL5 or elongation of very long chain fatty acids protein 5. ELOVL5 has been previously reported to be linked to insulin resistance and glaucoma, and a SNP has been reported in the 3′ UTR of ELOVL5.

Another identified case corresponds to the transcript MALAT1 also known as NEAT2, which is a non-coding transcript on chromosome 11. It was also determined that higher levels of short RNAs were generated from across the span of MALAT1/NEAT2 in DCIS breast samples, but not in normal breast samples, and the amount of the short RNAs subsequently decreases substantially in invasive breast cancer samples. This is a particularly notable example as it demonstrates that the findings made herein readily extend to non-coding transcripts (i.e. non-protein-coding RNAs, also known as non-coding RNAs) where the presence or absence of one or more short RNAs originating in a region that normally gives rise to a non-coding RNA transcript can be indicative of the emergence of a new state for the cell type or tissue at hand.

By analyzing the short RNA datasets, it was also determined that eIF4EBP2 gene (responsible for inhibition of translation) produces a high amount of short RNAs from its 3′ UTR region and also from one of its protein-coding exons in DCIS samples, but less in invasive or normal samples. In addition, eIF4EBP1 and eIF4EBP3 do not appear to be generating short RNAs out of their loci.

In addition, it was determined that SLC26A2 generated a high level of short RNAs out of its 3′ UTR and also from one of its protein-coding exons in DCIS samples, but not in invasive or normal samples. SLC7A2 also generated a high amount of short RNAs from nearly all of its protein-coding exons and its 3′ UTR in DCIS samples, but lower amounts in normal or invasive samples.

Other additional genes that generated a high amount of short RNAs out of their one or more exons in DCIS samples, but not in normal or invasive samples can include, but are not limited to, DSP (desmoplakin, which is linked to desmosomes and desmosomes is linked to cancer), SRRM2, HIPK2, AHNAK, ESR1, RUNX1, BCL2, MIA3, RHOB, ERBB2, PGR, IGF1R, and FASN. Among them, FASN appeared to generate short RNAs along its entire length of the assessed exon(s).

The amount of the short RNA transcripts as described herein and/or the exact location of their source in the mRNA depended on the state of a disease (e.g. normal breast sample vs. ductal-in-situ-carcinoma breast sample vs. invasive-cancer breast sample) or disorder, thus indicating diagnostic and prognostic roles for these non-coding short RNA molecules.

More importantly, the analyses of deep sequencing read sets from different tissues (e.g., pancreatic samples from normal and disease or disorder states, platelet samples from normal and disease or disorder states, and breast samples from normal and disease or disorder states) indicate that the correlation of the amount of the short RNAs produced from the exons of protein-coding genes to a specific state of a disease or disorder is a more general phenomenon, and thus the methods described herein can be extended to various types of tissues and to various collections of protein-coding genes.

Additionally, it was discovered that in several instances the mRNAs that give rise to the short non-coding RNA molecules generally correspond to genes whose protein products are typically known to be functionally significant for the corresponding tissue and state. Accordingly, any mRNAs that give rise to short non-coding RNA molecules can represent novel candidates as a biomarker for diagnosing a specific state of the corresponding tissue. Additionally, the discovery indicates that the remainder of the mRNAs that give rise to short non-coding RNA molecules correspond to currently unsuspected genes whose protein products are functionally significant for the corresponding tissue and state.

### Example 2

### Determination of a State/Condition of a Breast Tissue Sample Based on Detecting Short RNAs Produced from One or More Exons of ELOVL5

This example shows the amount of short RNAs present in the last exon of the gene known as ELOVL5 (elongation of very long chain fatty acids protein 5) from different breast samples, including 2 normal (Breast_{—}1N1 and Breast_{—}2N2), 1 ductal in situ carcinoma (Breast_{—}1D1) and 1 invasive (Breast_{—}2D2). The last exon of the ELOVL5 gene includes the gene's 3′UTR. ELOVL5 is located on the reverse strand (going from 3′ to 5′) of the human genome, as indicated in FIG. 1 by left-pointing arrowheads (i.e. <<<<<<<<) at the top right of FIG. 1 and the use of red bars to mark the location where the sequenced reads (corresponding to short RNAs) are mapped. In the top part of FIG. 1, exons are indicated by solid color rectangles separated by intronic regions that are indicated by long lines with arrowheads (i.e., [symbol] ). Sequenced reads that map to the same genomic location contribute independently to that location: the height of the red bar at a given genomic location represents the number of overlapping sequenced reads that map there. Note that the Y-axis is logarithmic (base 2) and ranges from 0 (0 reads) to 26 (2²⁶ reads). As shown in FIG. 1, for both normal samples (1N1 and 2N2) and the invasive samples (2D2), only a few sequenced reads (corresponding to short RNAs) were detected from a few locations across the ELOVL5 gene's last exon. However, the situation is markedly different in the ductal in situ carcinoma sample (1D1) where significantly more RNA molecules were produced from numerous locations along the last exon of the ELOVL5 gene.

### Example 3

### Determination of a State/Condition of a Breast Tissue Sample Based on Detecting Short RNAs Produced from One or More Exons of ESR1

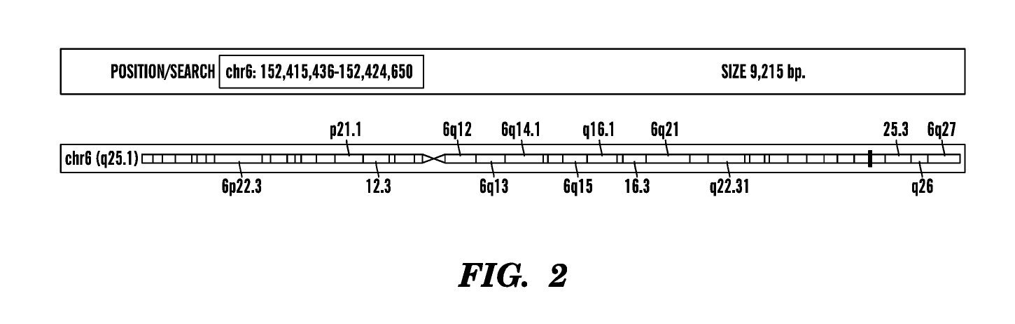

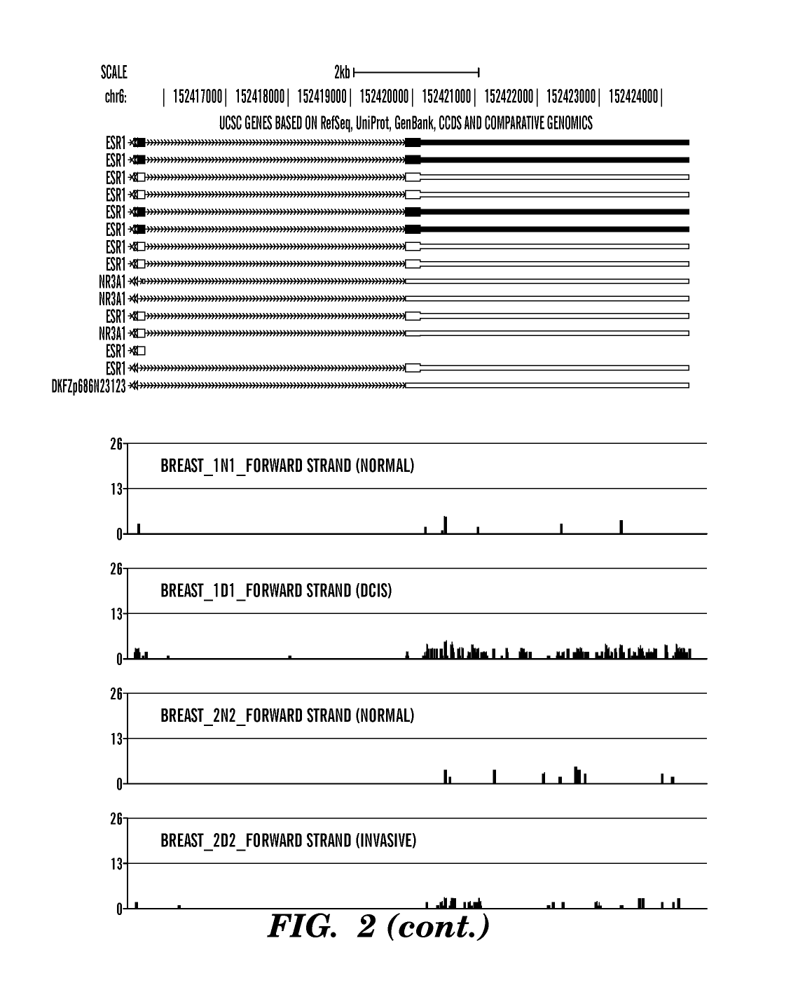

This example shows the amount of short RNAs present in the last two exons of the gene known as ESR1 (estrogen receptor 1) from different breast samples, including 2 normal (Breast_{—}1N1 and Breast_{—}2N2), 1 ductal in situ carcinoma (Breast_{—}1D1) and 1 invasive (Breast_{—}2D2). The last exon includes the gene's 3′UTR. ESR1 is located on the forward strand (going from 5′ to 3′) of the human genome, as indicated by right-pointing arrowheads (i.e. >>>>>>>>) at the top left of FIG. 2 and the use of blue bars to mark the location where the sequenced reads (corresponding to short RNAs) have mapped. In the top part of FIG. 2, exons are indicated by solid color rectangles separated by intronic regions that are indicated by long lines with arrowheads (i.e., [symbol] ). Sequenced reads that map to the same genomic location contribute independently to that location: the height of the blue bar at a given genomic location represents the number of overlapping sequenced reads that map there. Note that the Y-axis is logarithmic (base 2) and ranges from 0 (0 reads) to 26 (2²⁶ reads). As shown in FIG. 2, for both normal samples (1N1 and 2N2) and the invasive samples (2D2), only a few sequenced reads (corresponding to short RNAs) were detected from some locations across the ESR1 gene's last two shown exons. However, the situation is markedly different in the ductal in situ carcinoma sample (1D1) where significantly more RNA molecules were produced from numerous locations along the two shown exons of the ESR1 gene.

### Example 4

### Determination of a State/Condition of a Breast Tissue Sample Based on Detecting Short RNAs Produced from One or More Exons of SRRM2

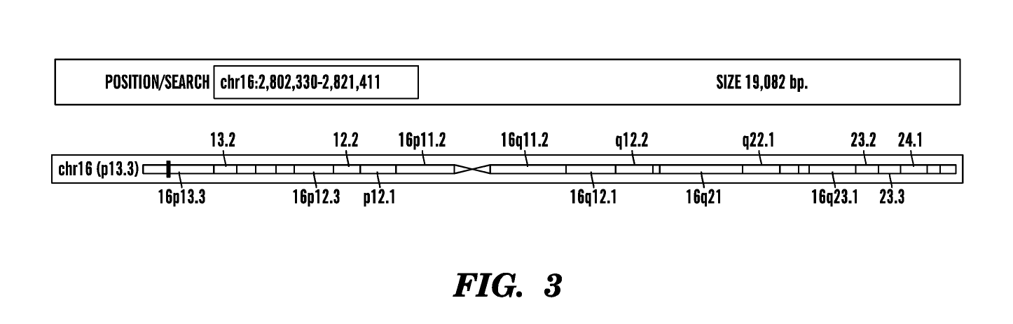

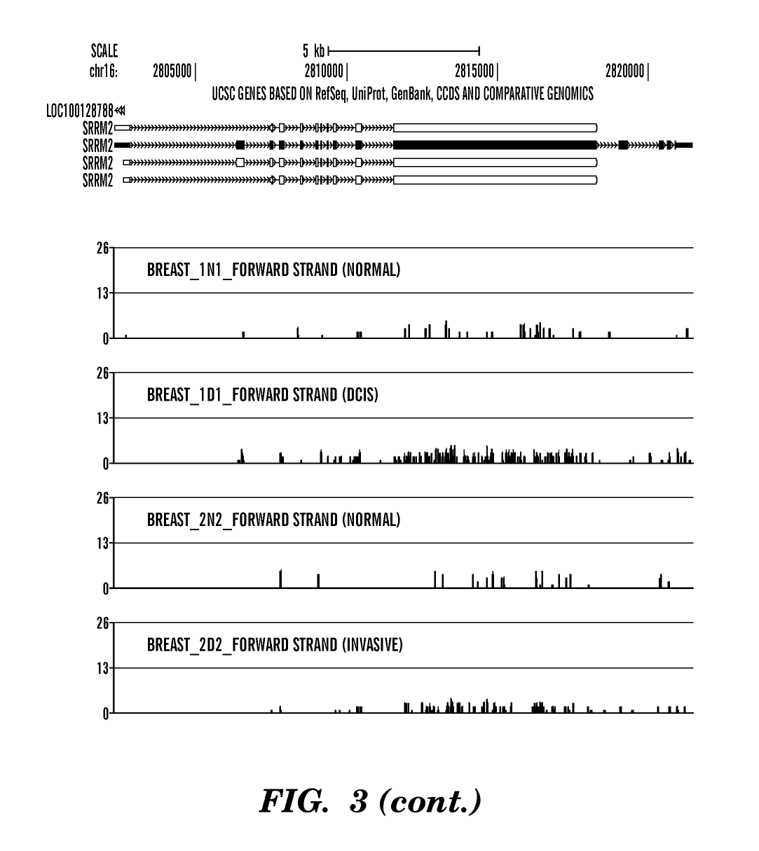

This example shows the amount of short RNAs present in several exons of the gene known as SRRM2 (serine/arginine repetitive matrix 2) from different breast samples, including 2 normal (Breast_{—}1N1 and Breast_{—}2N2), 1 ductal in situ carcinoma (Breast_{—}1D1) and 1 invasive (Breast_{—}2D2). SRRM2 is located on the forward strand (going from 5′ to 3′) of the human genome, as indicated by FIG. 3 by right-pointing arrowheads (i.e. >>>>>>>>) at the top left of FIG. 3 and the use of blue bars to mark the location where the sequenced reads (corresponding to short RNAs) have mapped. In the top part of FIG. 3, exons are indicated by solid color rectangles separated by intronic regions that are indicated by long lines with arrowheads (i.e., [symbol] ). Sequenced reads that map to the same genomic location contribute independently to that location: the height of the blue bar at a given genomic location represents the number of overlapping sequenced reads that map there. Note that the Y-axis is logarithmic (base 2) and ranges from 0 (0 reads) to 26 (2²⁶ reads). As shown in FIG. 3, for both normal samples (1N1 and 2N2), only a few sequenced reads (corresponding to short RNAs) were detected from some locations across the shown exons. However, the situation is markedly different in the ductal in situ carcinoma sample (1D1) where significantly more RNA molecules were produced from numerous locations along the exons of the SRRM2 gene. Similarly, in the invasive cancer sample (2D2), there are also more short RNAs produced than in the two normal samples (1N1 and 2N2).

### Example 5

### Determination of a State/Condition of a Breast Tissue Sample Based on Detecting Short RNAs Produced from One or More Exons of AHNAK

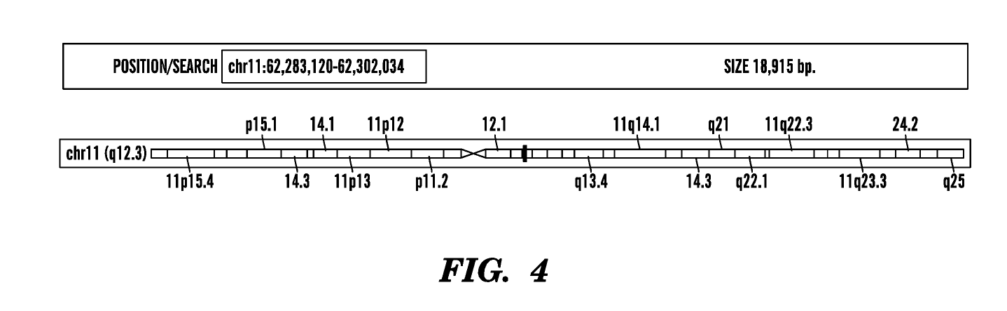

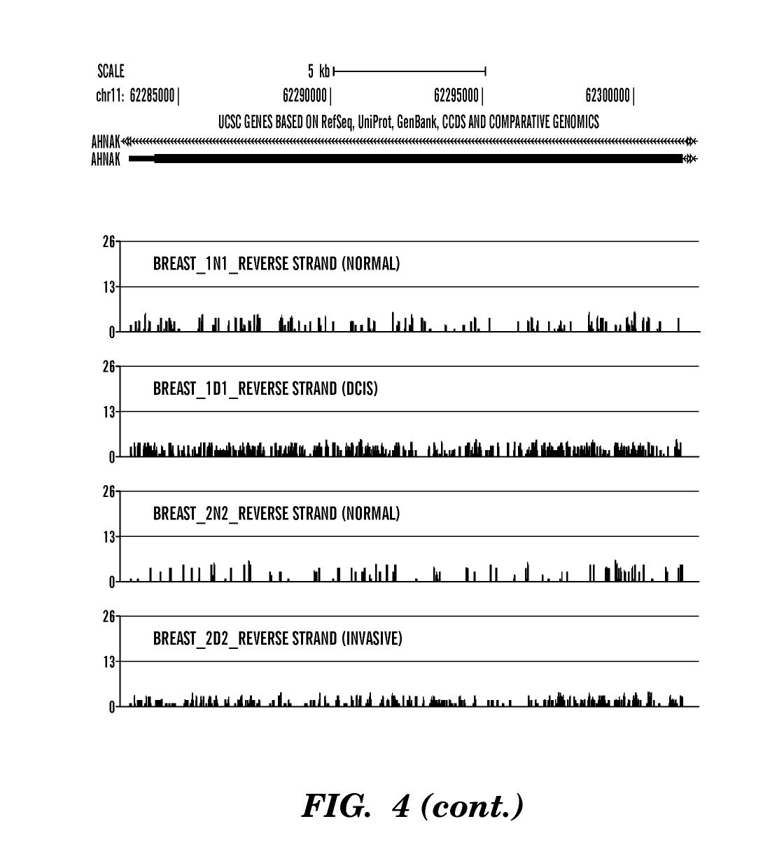

This example shows the amount of short RNAs present in an exon of the gene known as AHNAK or AHNAK-1 (AHNAK nucleoprotein) from different breast samples, including 2 normal (Breast_{—}1N1 and Breast_{—}2N2), 1 ductal in situ carcinoma (Breast_{—}1D1) and 1 invasive (Breast_{—}2D2). AHNAK is located on the reverse strand (going from 3′ to 5′ direction) of the human genome, as indicated in FIG. 4 by left-pointing arrowheads (i.e. <<<<<<<<) at the top right of FIG. 4 and the use of red bars to mark the location where the sequenced reads (corresponding to short RNAs) have mapped. In the top part of FIG. 4, exons are indicated by solid color rectangles separated by intronic regions that are indicated by long lines with arrowheads (i.e., [symbol] ). Sequenced reads that map to the same genomic location contribute independently to that location: the height of the red bar at a given genomic location represents the number of overlapping sequenced reads that map there. Note that the Y-axis is logarithmic (base 2) and ranges from 0 (0 reads) to 26 (2²⁶ reads). As shown in FIG. 4, for both normal samples (1N1 and 2N2), comparatively few sequenced reads (corresponding to short RNAs) were detected from the exon. However, the situation is markedly different in the ductal in situ carcinoma sample (1D1) and the invasive sample (2D2) where significantly more RNA molecules were produced from numerous locations along the exon. Similar trends can also be observed in pancreatic tissue samples, where significantly more short RNAs were produced from numerous locations along the exon of the AHNAK gene in the pancreatic cancer samples, while relatively few sequenced reads were detected in normal samples (Data not shown).

### Example 6

### Determination of a State/Condition of a Pancreatic Tissue Sample Based on Detecting short RNAs produced from one or more exons of CEL

In Examples 2-5, the presence (and/or an increase in amount) of short RNA transcripts sourced from one or more exons of a gene, as compared to normal samples, is indicative of a disease or abnormal state. However, the opposite can also be applicable, i.e., the absence (and/or decrease in amount) of short RNA transcripts sourced from one or more exons of a gene can be an indicator of a disease or abnormal state, as shown in the following Examples.

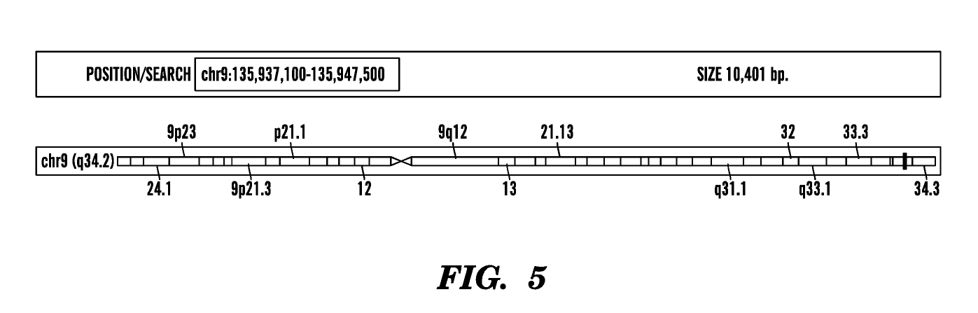

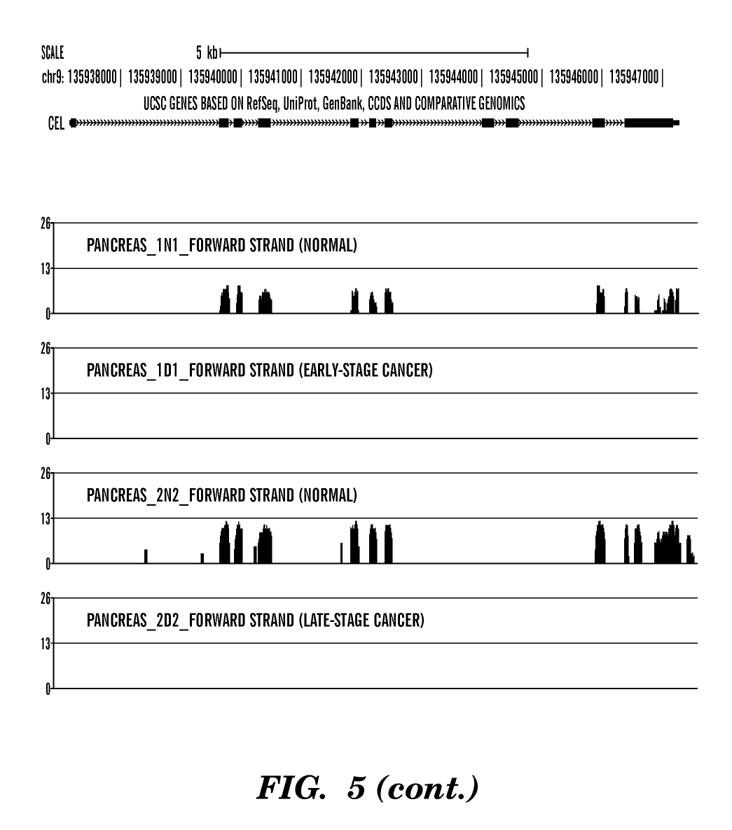

This example shows the amount of short RNAs present in the set of exons of the gene known as CEL (carboxyl ester lipase) from four pancreatic samples including 2 normal (Pancreas_{—}1N1 and Pancreas_{—}2N2), 1 early stage (Pancreas_{—}1D1) and 1 late stage (Pancreas_{—}2D2). CEL is located on the forward strand (going from 5′ to 3′ direction) of the human genome, as indicated in FIG. 5 by right-pointing arrowheads (i.e. >>>>>>>>) at the top left of FIG. 5 and the use of blue bars to mark the location where the sequenced reads (corresponding to short RNAs) have mapped. In the top part of FIG. 5, exons are indicated by solid color rectangles separated by intronic regions that are indicated by long lines with arrowheads (i.e., [symbol] ). Sequenced reads that map to the same genomic location contribute independently to that location: the height of the blue bar at a given genomic location represents the number of overlapping sequenced reads that map there. Note that the Y-axis is logarithmic (base 2) and ranges from 0 (0 reads) to 26 (2²⁶ reads). As shown in FIG. 5, for both normal samples (1N1 and 2N2), numerous sequenced reads (corresponding to short RNAs) were detected from across the shown exons of the CEL gene. However, the situation is markedly different in the early stage cancer (1D1) and the late stage cancer (2D2) samples where there is apparent absence (or presence at an undetectable level) of these short RNA molecules.

### Example 7

### Determination of a State/Condition of a Pancreatic Tissue Sample Based on Detecting Short RNAs Produced from One or More Exons of GP2

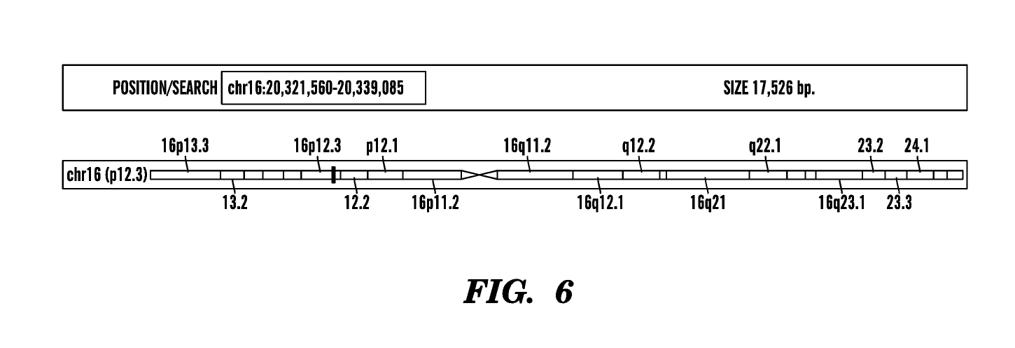

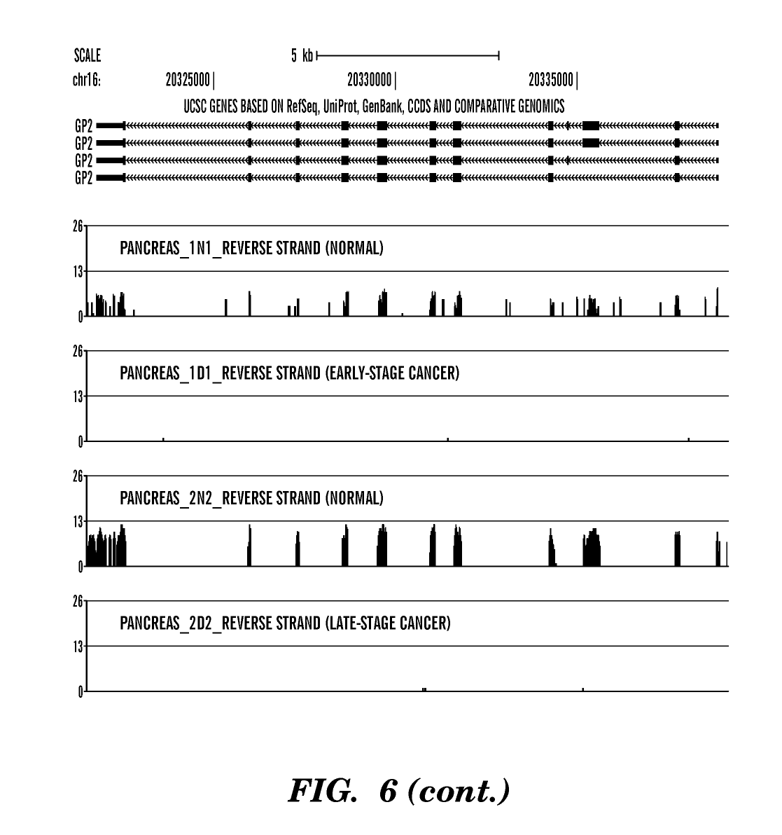

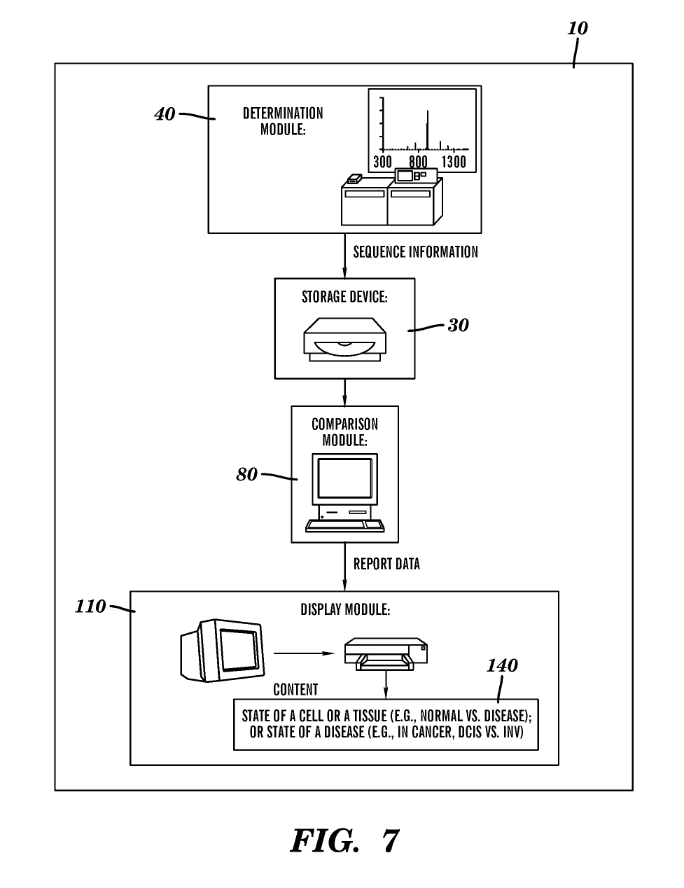

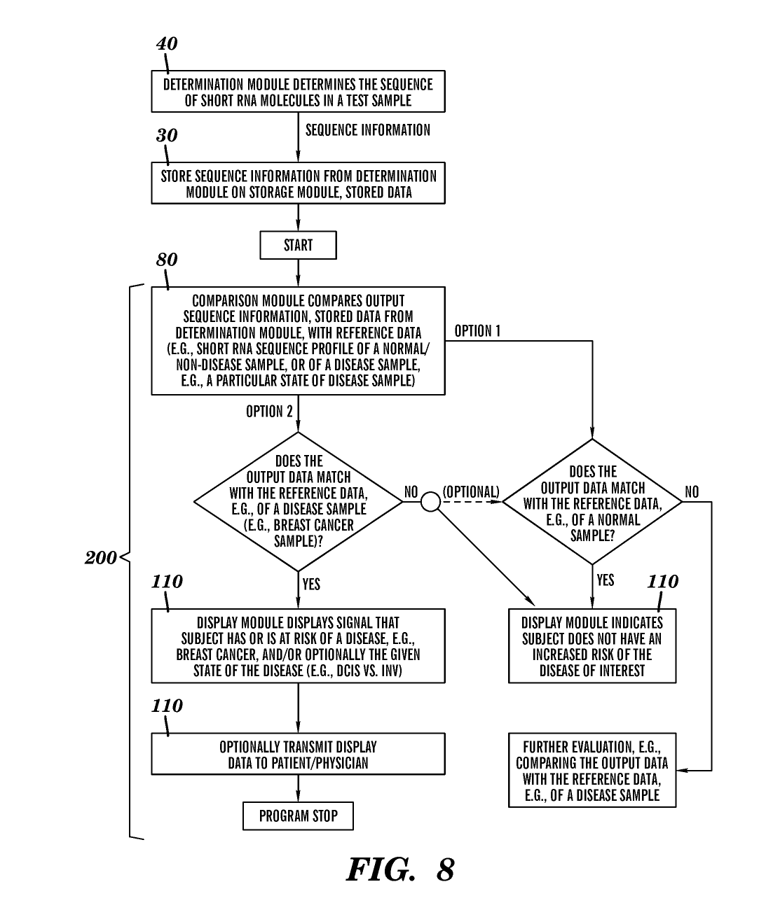

This example shows the amount of short RNAs present in the set of exons of the gene known as GP2 (glycoprotein 2 a.k.a. zymogen granule membrane) from four pancreatic samples including 2 normal (Pancreas_{—}1N1 and Pancreas_{—}2N2), 1 early stage (Pancreas_{—}1D1) and 1 late stage (Pancreas_{—}2D2). GP2 is located on the reverse strand (going from 3′ to 5′ direction) of the human genome, as indicated in FIG. 6 by left-pointing arrowheads (i.e. <<<<<<<<) at the top of FIG. 6 and the use of red bars to mark the location where the sequenced reads have mapped. In the top part of FIG. 6, exons are indicated by solid color rectangles separated by intronic regions that are indicated by long lines with arrowheads (i.e., [symbol] ). Sequenced reads that map to the same genomic location contribute independently to that location: the height of the red bar at a given genomic location represents the number of overlapping sequenced reads that map there. Note that the Y-axis is logarithmic (base 2) and ranges from 0 (0 reads) to 26 (2²⁶ reads). As shown in FIG. 6, in both normal samples (1N1 and 2N2), a number of sequenced reads (corresponding to short RNAs) were detected from each of GP2's exons. However, the situation is markedly different in the early stage cancer (1D1) and the late stage cancer (2D2) samples where there is apparent absence (or presence at an undetectable level) of these short RNA molecules.

### Example 8

### Detection of Short RNAs of ELOVL5 in Human Cell Lines

As shown in Example 2 and FIG. 1, for both normal samples (1N1 and 2N2) and invasive samples (2D2), only a few sequenced reads (corresponding to short RNAs) were detected from a few locations across the ELOVL5 gene's last exon, while significantly more RNA molecules were produced from numerous locations along the last exon of the ELOVL5 gene in the ductal in situ carcinoma sample (1D1). It was also determined that many exons of ELOVL5 exhibit a similar behavior as shown in FIG. 1, namely the number of sequenced reads (and thus number of generated RNA molecules) from the corresponding loci is markedly increased in the ductal in situ carcinoma sample (Data not shown).

To further verify the findings, experiments with human cell lines in an independent setting were performed. For example, Taqman qRT-PCR primers were designed for two regions of ELOVL5 that were represented by several sequenced reads (corresponding to short RNAs) in the deep-sequencing samples. One of the regions was selected from the ELOVL5 gene's 3′UTR whereas the second one was selected from a protein-coding exon of ELOVL5.

While other commercial primers can be used, Taqman primers were used in this Example. The Taqman assay has the ability to quantify the amount of the molecule being probed while ensuring, at the same time, that the assay will only amplify an RNA molecule if and only if it corresponds to the sequence being probed. The exemplary sequences of the two short RNA molecules, respectively, located in the 3′ UTR (B1) and the CDS (B2) regions are indicated below:

| (SEQ ID NO. 1) | |
|---|---|
| B1 (3′UTR): | AAATCTAGTGGAACAGTCAGTTTAACTTTTTAAC |
| (SEQ ID NO. 2) | |
| B2 (CDS): | TTACTATGGTTTGTCGTCAGTCCCTTCCATGCGT |

These two exemplary short RNA molecules have the same length of 34 nucleotides. Based on the state of the art, there have been no short RNA molecular classes reported with this length (i.e., ˜34 nucleotides). The representative members of the classes of miRNAs and piRNAs that have been reported and discussed in the art to date comprise molecules with lengths between 22 and 30 nucleotides.

B1 (i.e., a short RNA molecule produced from 3′ UTR of the ELOVL5 gene) was detected and identified in the breast cancer cell lines including, but not limited to, MCF10A, hDCIS, MDA-MB-231 and MDA-MB-468. B1 was also detected and identified in the pancreatic cancer cell lines including, but not limited to, MiaPaCa2 and N-90708N1. All experiments were run in triplicate.

B2 (i.e., a short RNA molecule produced from a protein-coding exon of the ELOVL5 gene) was detected and identified in the breast cancer cell lines including, but not limited to, MCF10A, hDCIS, MDA-MB-231 and MDA-MB-468. B2 was also detected and identified in the pancreatic cancer cell lines including, but not limited to, HPNE, MiaPaCa2 and PL-5. All experiments were run in triplicate.

### Example 9

### Diagnostic Applications

Genes including, but not limited to, ELOVL5, ESR1, AHNAK, CELL, GP2, and others, were determined to produce short RNAs out of their exons whose abundance and/or locations are indicative of the state of the tissue from which the sample was obtained. Non-coding transcripts including, but not limited to, MALAT1/NEAT2, were also determined to produce short RNAs out of the locus that typically gives rise to the known (long) transcript, and the abundance and/or locations of these short RNAs are indicative of the state of the tissue from which the sample was obtained. The presence or absence of the short RNAs can represent a “causal event” or a “result” of the tissue having entered a given state (e.g., a given state of a disease or disorder). If the amount of the short RNAs represents a “causal event” for a certain disease or disorder, then being able to ascertain that these short RNAs are present or absent can permit diagnosis of the state of the tissue in which these short RNAs are sought. One can envision a setting where one employs an exome capture array with probes representing genomic regions of interest, such as those described above that have been determined to give rise to short RNAs in a state-dependent manner, including but not limited to ELOVL5, ESR1, AHNAK, CELL, GP2, MALAT1/NEAT2 and others. The regions to be represented on such an array are expected to be a function of the application context. For example, in some embodiments, the probes placed on the array that represent a region of interest can be designed (e.g. based on prior profiling of the short RNA population) to specifically capture one or more of the short RNAs arising from the region of interest. In some embodiments, the probes placed on the array can be designed to represent some or all of the span of the genomic region of interest (“tiling” probes). In some embodiments, the probes placed on the array can be a combination of specific and tiling probes.

For a given sample to be examined, in one embodiment, total RNA can be extracted using known methods in the art; then, the short RNA populations in the total RNA can be enriched using known methods in the art; then, the resulting sub-population of (short) RNAs can be reverse transcribed into the corresponding cDNAs which are then allowed to hybridize with the designed array. Without wishing to be bound by theory, in some embodiments, since the RNAs have been size-selected, the presence or absence of hybridization to the array can indicate the presence or absence of these short RNAs: for example, no hybridization to the potentially longer mRNA could occur as longer molecules were excluded via size selection.

### Example 10

### Therapeutic Applications—Control of the Amount of the Short RNAs

Genes including, but not limited to, ELOVL5, ESR1, PGR, MALAT1/NEAT2 and others, were determined to produce more short RNAs out of their exons in DCIS samples than in invasive samples. This indicates that those short RNAs may be responsible for downstream functional effects.

Without wishing to be bound by theory, the amount of these short RNAs can be linked and/or correlated to the amount of the corresponding messenger RNA that is made up of the exons from which these short RNAs arise. Alternatively, the amount of these short RNAs can have no linkage or correlation to the amount of the corresponding messenger RNA that is made up of the exons from which these short RNAs arise. If the amount of the short RNAs is independent of the amount of the corresponding mRNA or of the corresponding non-coding RNA, which would indicate the involvement of additional unknown molecules, a therapeutic intervention can involve the simultaneous change in amount of mRNA or non-coding RNA in a given disease or disorder, and the change in amount of the short RNAs that originate from the mRNA or non-coding RNA that is being affected. On the other hand, if there is a correlation between the amount of an mRNA or non-coding RNA of interest and the amount of the short RNAs originating in the mRNA or non-coding RNA, a single-prong therapeutic intervention can be sufficient.

The amount of the short RNAs can represent a “causal event” or a “result” of the tissue having entered a given state (e.g., a given state of a disease or disorder). If the amount of the short RNAs represents a “causal event” for a certain disease or disorder, the amount of the short RNAs can be controlled to return their levels to what would be considered “normal” levels and thus alleviate the impact that can result from the changes in their amount. Examples of the techniques that can be used to control the amount of the short RNAs include, but are not limited to, antisensing or sponging (e.g., microRNA sponges as described in Ebert and Sharp. “MicroRNA sponges: Progress and possibilities” RNA (2010) 16:2043-2050; and Ebert et al. “MicroRNA sponges: Competitive inhibitors of small RNAs in mammalian cells” Nat. Methods (2007) 4: 721-726), decoying (e.g., as described in Swami M. “Small RNAs: Pseudogenes act as microRNA decoys.” Nature Reviews Genetics (2010) 11: 530-531), overexpression, and/or any art-recognized techniques.

SEQUENCE LISTING:

AAATCTAGTGGAACAGTCAGTTTAACTTTTTAAC (SEQ ID NO. 1)
TTACTATGGTTTGTCGTCAGTCCCTTCCATGCGT (SEQ ID NO. 2)
ATGTGAAATCAGACACGGCACCTTCA (SEQ ID NO: 3)
AAATCTAGTGGAACAGTCAGTTTAACTTTTTAAC (SEQ ID NO: 4)
ATTTGAGGCAGTGGTCAAACAGGTAAAGC (SEQ ID NO: 5)
TATGAGTTGTGCCCCAATGC (SEQ ID NO: 6)
TACAATGTTGTTATGGTAGAGAAACACACATGCC (SEQ ID NO: 7)
CTATTGGCTTTGAATCAAGCAGGCTC (SEQ ID NO: 8)
TGTATGTCTTCATTGCTAGG (SEQ ID NO: 9)
TCCAAACCACGTCATCTGATTGTAAGCA (SEQ ID NO: 10)
GCCTATGATGTGTGTCATTTTAAAGTGTCGGA (SEQ ID NO: 11)
CACGTCATCTGATTGTAAGCAC (SEQ ID NO: 12)
AAGCTGCGGAAGGATTGAAGTCAAAGAATT (SEQ ID NO: 13)
TAAAGCCTATGATGTGTGTCATTT (SEQ ID NO: 14)
GGGTCTAAATTTGGATTGATTTATGCAC (SEQ ID NO: 15)
AGATTTCTAACATTTCTGGGCTCTCTGACC (SEQ ID NO: 16)
AAGCAAAGTGTAAATCAGAGGTTTAAGTTAAAAT (SEQ ID NO: 17)
TGATTCATGTAGGACTTCTTTCATCAATTCAAAA (SEQ ID NO: 18)
GTGTCATTTTAAAGTGTCGGAATTTAGCCTCT (SEQ ID NO: 19)
GTGGGTTTTCTGTTTGAAAAGGAG (SEQ ID NO: 20)
GACACGGCACCTTCAGTTTTGTACTAT (SEQ ID NO: 21)
CATAAGAGAATCGAGAAATTTGATAGAGGT (SEQ ID NO: 22)
CAGCATAAGAGAATCGAGAAA (SEQ ID NO: 23)
AAGCTTATTAGTTTAAATTAGGGTATGTTTC (SEQ ID NO: 24)
TGTCTAAACAGTAATCATTAAAACATTTTTGATT (SEQ ID NO: 25)
TAGACTGCTTATCATAAAATCACATC (SEQ ID NO: 26)
CTTAGCTCACCTGGATATAC (SEQ ID NO: 27)
CGTAGATGAGCAATGGGGAAC (SEQ ID NO: 28)
ATGTAGGACTTCTTTCATCAATTCAAAACC (SEQ ID NO: 29)
ATGCTTTAATTTTGCACATTCGTACTATAGGGAG (SEQ ID NO: 30)
ATAAGATTTCTAACATTTCTGGGCTCTCTGACCC (SEQ ID NO: 31)
AGGTAAAATCAAATATAGCTACAGC (SEQ ID NO: 32)
AGAGATGATTGCCTATTTACC (SEQ ID NO: 33)
AACCCCTAGAAAACGTATAC (SEQ ID NO: 34)
AACATTTCTGGGCTCTCTGACCCCTGCG (SEQ ID NO: 35)
TTATCATAAAATCACATCTCACACATTTGAGGC (SEQ ID NO: 36)
TGGATATACCTACATTGTTAAATGTC (SEQ ID NO: 37)
TGCTTTAATTTTGCACATTCGTACTATAGGGAGCC (SEQ ID NO: 38)
GGGTCTAAATTTGGATTGATTTATGC (SEQ ID NO: 39)
GGCACCTTCAGTTTTGTACTATTGGCTTTGAATC (SEQ ID NO: 40)
GCACCTTCAGTTTTGTACTATTGGCTTTGAATCAA (SEQ ID NO: 41)
CGTCATCTGATTGTAAGCACAATATGAGTTGTGCC (SEQ ID NO: 42)
CCTCCAAACCACGTCATCTGATTGTAAGCACAAT (SEQ ID NO: 43)
ACATTTCTGGGCTCTCTGACCCC (SEQ ID NO: 44)
AACCCCTAGAAAACGTA (SEQ ID NO: 45)
TTTAGAAAAAATCAAAGACCATGATTTATGAAAC (SEQ ID NO: 46)
TCGTGATGAAACTTAAATATATATTCTTTGTC (SEQ ID NO: 47)
GTGTGATTCATGTAGGACTTC (SEQ ID NO: 48)
GGGCTCTACAGCAGTCGTGATGAAACTTAAATAT (SEQ ID NO: 49)
GCCTTAAAATTTAAAAAGCAGGGCCCAAAGCTTA (SEQ ID NO: 50)
GCCTTAAAATTTAAAAAGCAGGGCCCAAAGC (SEQ ID NO: 51)
GCACCTTCAGTTTTGTACTATTGGCTTTGAATCA (SEQ ID NO: 52)
GAAAGGGAGTATTATTATAGTATAC (SEQ ID NO: 53)
CTCACACATTTGAGGCAGTGG (SEQ ID NO: 54)
ATAGTACTTGTAATTTCTTTCTGCTTAGAATC (SEQ ID NO: 55)
AGGTAAAATCAAATATAACTACAGC (SEQ ID NO: 56)
AGATTTCCTTGTAAAATGTG (SEQ ID NO: 57)
ACCACGTCATCTGATTGTAAGC (SEQ ID NO: 58)
ACAGGTAAAGCCTATGATGTGTGT (SEQ ID NO: 59)
AATATGAGTTGTGCCCCAATGCTCG (SEQ ID NO: 60)
AACTAATGTGACATAATTTCCAGTGA (SEQ ID NO: 61)
TGGAAAGGGAGTATTATTATAGTATACAACACTG (SEQ ID NO: 62)
TGACTTGTTGATGTGAAATCAGACAC (SEQ ID NO: 63)
TACAGCATAAGAGAATCGAGAAATTTGATAGAGG (SEQ ID NO: 64)
GTTATAACATGATAGGTGCTGAATT (SEQ ID NO: 65)
GTAAATCTAATAGTACTTGTAATTTCTTTCTGCT (SEQ ID NO: 66)
GGTAAAGCCTATGATGTGTGTCATTTTAAAGTGTCG (SEQ ID NO: 67)
GGTAAAGCCTATGATGTGTGTCATTTTAAAGTGT (SEQ ID NO: 68)
GGGCTCTACAGCAGTCGTGATGAAACTTAAATATATATTCT (SEQ ID NO: 69)
GCGAGAGAGGATGTATACTTTTCAAGAGAGATGA (SEQ ID NO: 70)
CTAGTGGAACAGTCAGTTTAAC (SEQ ID NO: 71)
ATGGTAGAGAAACACACATGC (SEQ ID NO: 72)
ATGCTTTAATTTTGCACATTCGTACTATAGGGAGC (SEQ ID NO: 73)
ATCAATTCAAAACCCCTAGAAAACGTATACAG (SEQ ID NO: 74)
ATAAGATTTCTAACATTTCTGGGCTCTCTGACCCCT (SEQ ID NO: 75)
AGAAACACACATGCCTT (SEQ ID NO: 76)
ACCACGTCATCTGATTGTAAGCACAATATGAGTTC (SEQ ID NO: 77)
AAGCCTATGATGTGTGTCATTTTAAAGTGTCGGA (SEQ ID NO: 78)
AAATCTAGTGGAACAGTCAGTTTAACTTTTTAACAGA (SEQ ID NO: 79)
AAACCACGTCATCTGATTGTAAGC (SEQ ID NO: 80)

It is understood that the foregoing detailed description and examples are illustrative only and are not to be taken as limitations upon the scope of the invention. Various changes and modifications to the disclosed embodiments, which will be apparent to those of skill in the art, may be made without departing from the spirit and scope of the present invention. Further, all patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventor is not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A method of determining whether a subject has, or is at risk of developing, or is at a given stage of a condition afflicting a tissue of interest, the method comprising assaying a biological sample to measure expression level of one or more short RNA sequences originating from (a) at least one exon of a protein-coding gene, or from (b) at least one segment of a non-coding transcript, or from (c) both (a) and (b).

2. The method of claim 1, further comprising assaying the biological sample to measure expression levels of one or more short RNA sequences originating from (a) at least one exon of a plurality of protein-coding genes, or from (b) at least one segment of a plurality of non-coding transcripts, or from (c) both (a) and (b).

3. The method of claim 1, further comprising comparing the measured expression level of said one or more short RNA sequences with a reference level of a reference sample, wherein if the measured expression level of said one or more short RNA sequences deviates from the reference level, at least one cell present in the biological sample is determined to have a state, origin and/or cell type different from that of the reference sample; or if the measured expression level of said one or more the short RNA sequences is similar to the reference level, the biological sample is determined to have a similar state of the condition as represented by the reference sample, thereby determining whether a subject has, or is at risk of developing, or is at a given stage of the condition.

4. The method of claim 3, wherein the comparison further identifies an originating location of said one or more short RNA sequences from (a) said at least one exon of the protein-coding gene, or from (b) said at least one segment of the non-coding transcript, or from (c) both (a) and (b), wherein a discrepancy in the originating location of said one or more short RNA sequences in the biological sample from the reference sample is indicative of at least one cell present in the biological sample having a state, an origin and/or a cell type that is different from that of the reference sample, thereby determining whether a subject has, or is at risk of developing, or is at a given stage of the condition.

5. The method of claim 4, wherein the comparison further identifies an origin of the cell present in the biological sample.

6. The method of claim 1, wherein a plurality of the short RNA sequences are originated from (a) more than one exons of the protein-coding gene, or from (b) more than one segments of the non-coding transcript, or from (c) both (a) and (b).

7. The method of claim 1, wherein said one or more short RNA sequences have a length of about 10 nucleotides to about 40 nucleotides, or about 15 nucleotide to about 35 nucleotides, or about 17 nucleotides to about 30 nucleotides, or about 34 nucleotides.

8. The method of claim 3, wherein the reference sample represents a normal condition of a cell or tissue; or a recognizable stage of an abnormal condition of a cell or a tissue.

9. The method of claim 1, wherein the biological sample comprises one or more cells derived from the tissue of interest.

10. The method of claim 1, wherein the tissue of interest is selected from the group consisting of breast, pancreas, blood, prostate, colon, lung, skin, brain, liver, ovary, bone marrow, testis, and muscle.

11. The method of claim 1, wherein the condition is cancer.

12. The method of claim 11, wherein a comparison of the measured expression level of said one or more short RNA sequences with the reference level of the reference sample further identifies a primary origin of the cancer.

13. The method of claim 11, wherein when the cancer is breast carcinoma, the given stage of the condition to be determined comprises ductal in situ carcinoma, lobular in situ carcinoma, invasive breast carcinoma, or any combinations thereof.

14. The method of claim 13, wherein the protein-coding gene to be detected for determining the presence or absence of ductal in situ carcinoma is selected from the group consisting of ABCC11, ACTB, ACTG1, AHCY, AHNAK, ANKHD1, APP, ARF1, ASAH1, ATP1A1, ATP1B1, ATP6V0E1, AZGP1, B2M, B4GALT1, BAZ2A, BST2, BTG2, C1orf43, C3orf1, CALM2, CALR, CANX, CCNI, CD151, CD164, CD44, CD46, CD59, CD74, CEACAM6, CIRBP, CLIC6, COL1A2, COL6A1, COL6A3, COMMD3, COX7A2, CSDE1, CSRP1, CST3, CTNND1, CTSB, CXCL13, CYBRD1, DCN, DDX17, DDX5, DSP, DUSP4, EEF2, EFHD1, EHF, EIF4EBP3, EIF4G2, ELOVL5, ERBB2, ERBB3, ESR1, FASN, FAT1, FLNB, FMOD, FN1, FOXA1, FTL, GAPDH, GATA3, GDI2, GJA1, GLUL, HDLBP, HIST1H1B, HIST1H2AC, HIST1H3D, HIST1H4H, HNRNPF, HSP90AB1, IFI6, IGFBP4, IGHG4, ITGB4, JUP, KIAA0100, KIAA1522, LAPTM4A, LPHN1, LRBA, LRP2, MAGED2, MDH1, MED13L, MKNK2, MLL5, MLPH, MT-CO2, MUC1, MYB, MYH9, MYL6, NCL, NDUFA2, NET1, NF1, NME1, NUCKS1, P4HB, PACS2, PCBP2, PDCD4, PDIA3, PDLIM1, PDXDC1, PEG10, PFN1, PGR, PI15, PNRC1, PPDPF, PSMD5, PTPRF, QDPR, RARG, RBM39, RHOA, RHOB, RNF41, RPL15, S100A16, SEC11A, SERPINA1, SERPINA3, SFRP2, SH3BGRL, SIAH2, SLC25A6, SLC26A2, SLC38A1, SLC39A6, SLC7A2, SMG5, SREBF2, SRRM2, SSR2, STEAP1, STOM, TAGLN2, TAT, TFF3, TGOLN2, THAP4, TMBIM6, TMC5, TMED2, TMED5, TMEM59, TMEM66, TOB1, TPT1, TRPS1, TSPAN1, TTC39A, TUFM, TXNIP, UBN1, UBXN4, UFC1, UGDH, UNC13B, VIM, WAPAL, WIPI1, WNK1, XBP1, ZBTB7B, and any combinations thereof.

15. The method of claim 13, wherein the protein coding gene to be detected for determining the presence or absence of invasive breast carcinoma is selected from the group consisting of ABCC11, ACTB, ACTG1, ADAR, AFF3, AHCY, AHNAK, ANKHD1, APP, ARF1, ARHGDIB, ASAH1, ATP1A1, ATP1B1, ATP6V0E1, AZGP1, B2M, B4GALT1, BAZ2A, BST2, BTG2, C1orf43, C5orf45, CALM2, CALR, CANX, CCNI, CD151, CD164, CD44, CD46, CD59, CD74, CD81, CEACAM6, CELSR1, CELSR2, CEP350, CILP, CIRBP, CLDN4, CLIC6, COL1A2, COL3A1, COL6A3, COMMD3, COX7A2, CSDE1, CSRP1, CTNNA1, CTNNB1, CTSD, CXCL13, CYBRD1, DBI, DCN, DDX17, DDX5, DSP, DUSP4, EEF2, EFHD1, EHF, EIF4EBP3, EIF4G2, ELF3, ELOVL5, EPRS, ERBB2, ERBB3, ESR1, FASN, FHL2, FLNB, FMOD, FOXA1, FTH1, GAPDH, GATA3, GDI2, GJA1, GLUL, GNAS, GNB2L1, GSTK1, HDLBP, HIST1H1C, HIST1H1D, HIST1H1E, HIST1H2AC, HIST1H2AE, HIST1H2BC, HIST1H2BD, HIST1H3D, HIST1H4B, HIST1H4D, HIST1H4H, HIST2H2AB, HIST2H2AC, HIST4H4, HNRNPF, HSP90AA1, HSP90AB1, IFI6, IGFBP4, IGHG1, IGHG4, IGKC, JTB, JUP, KIAA0100, KIAA1522, KRT19, LAPTM4A, LMNA, LONP2, LPHN1, LRBA, MAGED2, MCL1, MDH1, MED13L, MGP, MKNK2, MLL5, MLPH, MPZL1, MT-CO2, MT-CYB, MUC1, MYB, MYH9, MYST3, NCL, NDUFA2, NDUFB5, NET1, NF1, NFIB, NME1, NUCKS1, OAZ1, P4HB, PACS2, PCBP2, PDCD4, PDIA3, PDLIM1, PDXDC1, PEG10, PFN1, PGR, PHB2, PI15, PNRC1, PPDPF, PRICKLE4, PSAP, PTPRF, QDPR, RARG, RBM39, RHOA, RHOB, RNF41, RPL13AP20, RPL15, RPL17, RPL4, RPLP2, RPS2, S100A11, S100A14, S100A16, S100A9, SAT1, SEMA3C, SERPINA1, SERPINA3, SF3B1, SGK3, SH3BGRL, SIAH2, SLC25A3, SLC25A6, SLC26A2, SLC38A1, SLC39A6, SLC7A2, SMG5, SPARC, SPTBN1, SREBF2, SRRM2, SSR2, STEAP1, STOM, TAGLN2, TAT, TBC1D16, TFF3, TGOLN2, THAP4, TM9SF2, TMBIM6, TMC5, TMED2, TMEM59, TMEM66, TOB1, TOMM6, TPT1, TRPS1, TSPAN1, TTC39A, TUFM, TXNIP, UCK2, UFC1, UGDH, UNC13B, WIPI1, WNK1, XBP1, ZBTB7B, ZNF207, and any combinations thereof.

16. The method of claim 11, wherein when the cancer is pancreatic cancer, the given stage of the condition to be determined includes an early stage pancreatic cancer, a late stage pancreatic cancer, or both.

17. The method of claim 16, wherein the protein coding gene to be detected for determining the presence or absence of the early stage pancreatic cancer is selected from the group consisting of ACTG1, ALB, AMY2B, C7, CEL, CELA3A, CLPS, COL3A1, CPA1, CPA2, CPB1, CTRB1, CTRB2, CUZD1, EEF2, GANAB, GATM, GP2, HDLBP, KHDRBS1, KLK1, KRT7, OLFM4, P4HB, PLA2G1B, PPDPF, PRSS1, PRSS3, REG1A, REG1B, REG3A, RNASE1, RPL8, SPINK1, SYCN, UNC13B, and any combinations thereof.

18. The method of claim 16, wherein the protein coding gene to be detected for determining the presence or absence of the late stage pancreatic cancer is selected from the group consisting of ACTB, ANXA2, ANXA5, APOE, ATP6VOC, C1QA, C1QB, C1QC, CIS, CALR, CCNI, CD14, CD44, CD59, CD68, COL1A2, COL6A3, CTSB, CTSC, EEF2, F13A1, FLNA, FN1, GLUL, GPNMB, GPX1, HIST1H2BD, IGFBP4, IGHM, IGKC, ISG15, LAMB3, LAPTM5, LGALS3BP, METTL7A, MMP11, MMP14, MT-CO2, MT-CYB, MYH9, OAZ1, P4HB, PLEC, PSAP, RNASE1, RPN1, SAT1, SERPINA1, SERPING1, SLC40A1, SLCO2B1, SPP1, SRGN, TGM2, TGOLN2, TIMP2, TXNIP, VSIG4, ZYX, and any combinations thereof.

19. The method of claim 13, wherein when the protein-coding gene to be detected for determining a given state of breast carcinoma comprises ELOVL5, at least a portion of said one or more short RNA sequences comprises a nucleotide sequence selected from the group consisting of SEQ ID NO: 1 to SEQ ID NO: 80, or a fragment thereof.

20. The method of claim 1, wherein the condition is a neurological disorder.

21. The method of claim 20, wherein the neurological disorder is selected from the group consisting of Parkinson's disease, Huntington's disease, Pick's disease, amyotrophic lateral sclerosis (ALS), dementia, Alzheimer's disease, and any combinations thereof.

22. The method of claim 1, wherein the exon comprises an untranslated region of the protein-coding gene.

23. The method of claim 1, wherein at least one of said one or more short RNA sequences has an overlapping region with a pyknon.

24. The method of claim 1, further comprising administering a treatment to the subject determined to have, or is at risk of developing, or is at a given stage of the condition.

25. The method of claim 1, wherein a comparison of the measured expression level of said one or more short RNA sequences with the reference level of the reference sample further identifies an origin of the biological sample.

26. A system for analyzing a biological sample comprising:
a) a determination module configured to receive a biological sample and to determine sequence information, wherein the sequence information comprises a sequence of a short RNA molecule originating from (i) an exon of at least one protein-coding gene, or from (ii) a segment of at least one non-coding transcript, or from (iii) both (i) and (ii);
b) a storage device configured to store sequence information from the determination module;
c) a comparison module adapted to compare the sequence information stored on the storage device with reference data, and to provide a comparison result, wherein the comparison result identifies the presence or absence of the short RNA molecule, wherein a discrepancy in an expression level or in an originating location of the short RNA molecule from the reference data is indicative of the biological sample having an increased likelihood of having or being at a cellular or tissue state different from a state represented by the reference data; and
d) a display module for displaying a content based in part on the comparison result for the user, wherein the content is a signal indicative of a subject having, or being at risk of developing, or being at a given stage of a disease or disorder, or a signal indicative of lack of a disease or disorder.


SEQUENCE LISTING (80 sequences)

SEQ ID NO: 1 - DNA, 34 bp, Unknown
  Description of Unknown Exemplary ELOVL5 oligonucleotide
aaatctagtggaacagtcagtttaactttttaac

SEQ ID NO: 2 - DNA, 34 bp, Unknown
  Description of Unknown Exemplary ELOVL5 oligonucleotide
ttactatggtttgtcgtcagtcccttccatgcgt

SEQ ID NO: 3 - DNA, 26 bp, Unknown
  Description of Unknown Exemplary ELOVL5 oligonucleotide
atgtgaaatcagacacggcaccttca

SEQ ID NO: 4 - DNA, 34 bp, Unknown; identical to SEQ ID NO: 1
  Description of Unknown Exemplary ELOVL5 oligonucleotide

SEQ ID NO: 5 - DNA, 29 bp, Unknown
  Description of Unknown Exemplary ELOVL5 oligonucleotide
atttgaggcagtggtcaaacaggtaaagc

SEQ ID NO: 6 - DNA, 20 bp, Unknown
  Description of Unknown Exemplary ELOVL5 oligonucleotide
tatgagttgtgccccaatgc

SEQ ID NO: 7 - DNA, 34 bp, Unknown
  Description of Unknown Exemplary ELOVL5 oligonucleotide
tacaatgttgttatggtagagaaacacacatgcc

SEQ ID NO: 8 - DNA, 26 bp, Unknown
  Description of Unknown Exemplary ELOVL5 oligonucleotide
ctattggctttgaatcaagcaggctc

SEQ ID NO: 9 - DNA, 20 bp, Unknown
  Description of Unknown Exemplary ELOVL5 oligonucleotide
tgtatgtcttcattgctagg

SEQ ID NO: 10 - DNA, 28 bp, Unknown
  Description of Unknown Exemplary ELOVL5 oligonucleotide
tccaaaccacgtcatctgattgtaagca

SEQ ID NO: 11 - DNA, 32 bp, Unknown
  Description of Unknown Exemplary ELOVL5 oligonucleotide
gcctatgatgtgtgtcattttaaagtgtcgga

SEQ ID NO: 12 - DNA, 22 bp, Unknown
  Description of Unknown Exemplary ELOVL5 oligonucleotide
cacgtcatctgattgtaagcac

SEQ ID NO: 13 - DNA, 30 bp, Unknown
  Description of Unknown Exemplary ELOVL5 oligonucleotide
aagctgcggaaggattgaagtcaaagaatt

SEQ ID NO: 14 - DNA, 24 bp, Unknown
  Description of Unknown Exemplary ELOVL5 oligonucleotide
taaagcctatgatgtgtgtcattt

SEQ ID NO: 15 - DNA, 28 bp, Unknown
  Description of Unknown Exemplary ELOVL5 oligonucleotide
gggtctaaatttggattgatttatgcac

SEQ ID NO: 16 - DNA, 30 bp, Unknown
  Description of Unknown Exemplary ELOVL5 oligonucleotide
agatttctaacatttctgggctctctgacc

SEQ ID NO: 17 - DNA, 34 bp, Unknown
  Description of Unknown Exemplary ELOVL5 oligonucleotide
aagcaaagtgtaaatcagaggtttaagttaaaat

SEQ ID NO: 18 - DNA, 34 bp, Unknown
  Description of Unknown Exemplary ELOVL5 oligonucleotide
tgattcatgtaggacttctttcatcaattcaaaa

SEQ ID NO: 19 - DNA, 32 bp, Unknown
  Description of Unknown Exemplary ELOVL5 oligonucleotide
gtgtcattttaaagtgtcggaatttagcctct

SEQ ID NO: 20 - DNA, 24 bp, Unknown
  Description of Unknown Exemplary ELOVL5 oligonucleotide
gtgggttttctgtttgaaaaggag

SEQ ID NO: 21 - DNA, 27 bp, Unknown
  Description of Unknown Exemplary ELOVL5 oligonucleotide
gacacggcaccttcagttttgtactat

SEQ ID NO: 22 - DNA, 30 bp, Unknown
  Description of Unknown Exemplary ELOVL5 oligonucleotide
cataagagaatcgagaaatttgatagaggt

SEQ ID NO: 23 - DNA, 21 bp, Unknown
  Description of Unknown Exemplary ELOVL5 oligonucleotide
cagcataagagaatcgagaaa

SEQ ID NO: 24 - DNA, 31 bp, Unknown
  Description of Unknown Exemplary ELOVL5 oligonucleotide
aagcttattagtttaaattagggtatgtttc

SEQ ID NO: 25 - DNA, 34 bp, Unknown
  Description of Unknown Exemplary ELOVL5 oligonucleotide
tgtctaaacagtaatcattaaaacatttttgatt

SEQ ID NO: 26 - DNA, 26 bp, Unknown
  Description of Unknown Exemplary ELOVL5 oligonucleotide
tagactgcttatcataaaatcacatc

SEQ ID NO: 27 - DNA, 20 bp, Unknown
  Description of Unknown Exemplary ELOVL5 oligonucleotide
cttagctcacctggatatac

SEQ ID NO: 28 - DNA, 21 bp, Unknown
  Description of Unknown Exemplary ELOVL5 oligonucleotide
cgtagatgagcaatggggaac

SEQ ID NO: 29 - DNA, 30 bp, Unknown
  Description of Unknown Exemplary ELOVL5 oligonucleotide
atgtaggacttctttcatcaattcaaaacc

SEQ ID NO: 30 - DNA, 34 bp, Unknown
  Description of Unknown Exemplary ELOVL5 oligonucleotide
atgctttaattttgcacattcgtactatagggag

SEQ ID NO: 31 - DNA, 34 bp, Unknown
  Description of Unknown Exemplary ELOVL5 oligonucleotide
ataagatttctaacatttctgggctctctgaccc

SEQ ID NO: 32 - DNA, 25 bp, Unknown
  Description of Unknown Exemplary ELOVL5 oligonucleotide
aggtaaaatcaaatatagctacagc

SEQ ID NO: 33 - DNA, 21 bp, Unknown
  Description of Unknown Exemplary ELOVL5 oligonucleotide
agagatgattgcctatttacc

SEQ ID NO: 34 - DNA, 20 bp, Unknown
  Description of Unknown Exemplary ELOVL5 oligonucleotide
aacccctagaaaacgtatac

SEQ ID NO: 35 - DNA, 28 bp, Unknown
  Description of Unknown Exemplary ELOVL5 oligonucleotide
aacatttctgggctctctgacccctgcg

SEQ ID NO: 36 - DNA, 33 bp, Unknown
  Description of Unknown Exemplary ELOVL5 oligonucleotide
ttatcataaaatcacatctcacacatttgaggc

SEQ ID NO: 37 - DNA, 26 bp, Unknown
  Description of Unknown Exemplary ELOVL5 oligonucleotide
tggatatacctacattgttaaatgtc

SEQ ID NO: 38 - DNA, 35 bp, Unknown
  Description of Unknown Exemplary ELOVL5 oligonucleotide
tgctttaattttgcacattcgtactatagggagcc

SEQ ID NO: 39 - DNA, 26 bp, Unknown
  Description of Unknown Exemplary ELOVL5 oligonucleotide
gggtctaaatttggattgatttatgc

SEQ ID NO: 40 - DNA, 34 bp, Unknown
  Description of Unknown Exemplary ELOVL5 oligonucleotide
ggcaccttcagttttgtactattggctttgaatc

SEQ ID NO: 41 - DNA, 35 bp, Unknown
  Description of Unknown Exemplary ELOVL5 oligonucleotide
gcaccttcagttttgtactattggctttgaatcaa

SEQ ID NO: 42 - DNA, 35 bp, Unknown
  Description of Unknown Exemplary ELOVL5 oligonucleotide
cgtcatctgattgtaagcacaatatgagttgtgcc

SEQ ID NO: 43 - DNA, 34 bp, Unknown
  Description of Unknown Exemplary ELOVL5 oligonucleotide
cctccaaaccacgtcatctgattgtaagcacaat

SEQ ID NO: 44 - DNA, 23 bp, Unknown
  Description of Unknown Exemplary ELOVL5 oligonucleotide
acatttctgggctctctgacccc

SEQ ID NO: 45 - DNA, 17 bp, Unknown
  Description of Unknown Exemplary ELOVL5 oligonucleotide
aacccctagaaaacgta

SEQ ID NO: 46 - DNA, 34 bp, Unknown
  Description of Unknown Exemplary ELOVL5 oligonucleotide
tttagaaaaaatcaaagaccatgatttatgaaac

SEQ ID NO: 47 - DNA, 32 bp, Unknown
  Description of Unknown Exemplary ELOVL5 oligonucleotide
tcgtgatgaaacttaaatatatattctttgtc

SEQ ID NO: 48 - DNA, 21 bp, Unknown
  Description of Unknown Exemplary ELOVL5 oligonucleotide
gtgtgattcatgtaggacttc

SEQ ID NO: 49 - DNA, 34 bp, Unknown
  Description of Unknown Exemplary ELOVL5 oligonucleotide
gggctctacagcagtcgtgatgaaacttaaatat

SEQ ID NO: 50 - DNA, 34 bp, Unknown
  Description of Unknown Exemplary ELOVL5 oligonucleotide
gccttaaaatttaaaaagcagggcccaaagctta

SEQ ID NO: 51 - DNA, 31 bp, Unknown
  Description of Unknown Exemplary ELOVL5 oligonucleotide
gccttaaaatttaaaaagcagggcccaaagc

SEQ ID NO: 52 - DNA, 34 bp, Unknown
  Description of Unknown Exemplary ELOVL5 oligonucleotide
gcaccttcagttttgtactattggctttgaatca

SEQ ID NO: 53 - DNA, 25 bp, Unknown
  Description of Unknown Exemplary ELOVL5 oligonucleotide
gaaagggagtattattatagtatac

SEQ ID NO: 54 - DNA, 21 bp, Unknown
  Description of Unknown Exemplary ELOVL5 oligonucleotide
ctcacacatttgaggcagtgg

SEQ ID NO: 55 - DNA, 32 bp, Unknown
  Description of Unknown Exemplary ELOVL5 oligonucleotide
atagtacttgtaatttctttctgcttagaatc

SEQ ID NO: 56 - DNA, 25 bp, Unknown
  Description of Unknown Exemplary ELOVL5 oligonucleotide
aggtaaaatcaaatataactacagc

SEQ ID NO: 57 - DNA, 20 bp, Unknown
  Description of Unknown Exemplary ELOVL5 oligonucleotide
agatttccttgtaaaatgtg

SEQ ID NO: 58 - DNA, 22 bp, Unknown
  Description of Unknown Exemplary ELOVL5 oligonucleotide
accacgtcatctgattgtaagc

SEQ ID NO: 59 - DNA, 24 bp, Unknown
  Description of Unknown Exemplary ELOVL5 oligonucleotide
acaggtaaagcctatgatgtgtgt

SEQ ID NO: 60 - DNA, 25 bp, Unknown
  Description of Unknown Exemplary ELOVL5 oligonucleotide
aatatgagttgtgccccaatgctcg

SEQ ID NO: 61 - DNA, 26 bp, Unknown
  Description of Unknown Exemplary ELOVL5 oligonucleotide
aactaatgtgacataatttccagtga

SEQ ID NO: 62 - DNA, 34 bp, Unknown
  Description of Unknown Exemplary ELOVL5 oligonucleotide
tggaaagggagtattattatagtatacaacactg

SEQ ID NO: 63 - DNA, 26 bp, Unknown
  Description of Unknown Exemplary ELOVL5 oligonucleotide
tgacttgttgatgtgaaatcagacac

SEQ ID NO: 64 - DNA, 34 bp, Unknown
  Description of Unknown Exemplary ELOVL5 oligonucleotide
tacagcataagagaatcgagaaatttgatagagg

SEQ ID NO: 65 - DNA, 25 bp, Unknown
  Description of Unknown Exemplary ELOVL5 oligonucleotide
gttataacatgataggtgctgaatt

SEQ ID NO: 66 - DNA, 34 bp, Unknown
  Description of Unknown Exemplary ELOVL5 oligonucleotide
gtaaatctaatagtacttgtaatttctttctgct

SEQ ID NO: 67 - DNA, 36 bp, Unknown
  Description of Unknown Exemplary ELOVL5 oligonucleotide
ggtaaagcctatgatgtgtgtcattttaaagtgtcg

SEQ ID NO: 68 - DNA, 34 bp, Unknown
  Description of Unknown Exemplary ELOVL5 oligonucleotide
ggtaaagcctatgatgtgtgtcattttaaagtgt

SEQ ID NO: 69 - DNA, 41 bp, Unknown
  Description of Unknown Exemplary ELOVL5 oligonucleotide
gggctctacagcagtcgtgatgaaacttaaatatatattct

SEQ ID NO: 70 - DNA, 34 bp, Unknown
  Description of Unknown Exemplary ELOVL5 oligonucleotide
gcgagagaggatgtatacttttcaagagagatga

SEQ ID NO: 71 - DNA, 22 bp, Unknown
  Description of Unknown Exemplary ELOVL5 oligonucleotide
ctagtggaacagtcagtttaac

SEQ ID NO: 72 - DNA, 21 bp, Unknown
  Description of Unknown Exemplary ELOVL5 oligonucleotide
atggtagagaaacacacatgc

SEQ ID NO: 73 - DNA, 35 bp, Unknown
  Description of Unknown Exemplary ELOVL5 oligonucleotide
atgctttaattttgcacattcgtactatagggagc

SEQ ID NO: 74 - DNA, 32 bp, Unknown
  Description of Unknown Exemplary ELOVL5 oligonucleotide
atcaattcaaaacccctagaaaacgtatacag

SEQ ID NO: 75 - DNA, 36 bp, Unknown
  Description of Unknown Exemplary ELOVL5 oligonucleotide
ataagatttctaacatttctgggctctctgacccct

SEQ ID NO: 76 - DNA, 17 bp, Unknown
  Description of Unknown Exemplary ELOVL5 oligonucleotide
agaaacacacatgcctt

SEQ ID NO: 77 - DNA, 35 bp, Unknown
  Description of Unknown Exemplary ELOVL5 oligonucleotide
accacgtcatctgattgtaagcacaatatgagttc

SEQ ID NO: 78 - DNA, 34 bp, Unknown
  Description of Unknown Exemplary ELOVL5 oligonucleotide
aagcctatgatgtgtgtcattttaaagtgtcgga

SEQ ID NO: 79 - DNA, 37 bp, Unknown
  Description of Unknown Exemplary ELOVL5 oligonucleotide
aaatctagtggaacagtcagtttaactttttaacaga

SEQ ID NO: 80 - DNA, 24 bp, Unknown
  Description of Unknown Exemplary ELOVL5 oligonucleotide
aaaccacgtcatctgattgtaagc